(19) United States
(12) Patent Application Publication
(10) Pub. No.: US 2018/0275146 A1
(43) Pub. Date: Sep. 27, 2018

(54) COMPOSITIONS AND METHODS FOR DIAGNOSIS AND TREATMENT OF PERVASIVE DEVELOPMENTAL DISORDER

(71) Applicant: Berg LLC, Nashville, TN (US)

(72) Inventors: Niven Rajin Narain, Cambridge, MA (US); Paula Patricia Narain, Cambridge, MA (US)

(21) Appl. No.: 15/830,982

(22) Filed: Dec. 4, 2017

Related U.S. Application Data
(63) Continuation of application No. 15/493,383, filed on Apr. 21, 2017, now abandoned.
Application No. 15/493,383 is a continuation of application No. 15/265,174, filed on Sep. 14, 2016, now abandoned.
Application No. 15/265,174 is a continuation of application No. 14/383,450, filed on Sep. 5, 2014, now abandoned.
(60) Provisional application No. 61/606,935, filed on Mar. 5, 2012.

(51) Int. Cl. G01N 33/68; C07K 16/18; A61K 38/48; A61K 38/17; A61K 38/45; C12Q 1/6883; A61K 38/44; A61K 38/46

(52) U.S. Cl. CPC G01N 33/6896; C07K 16/18; C12Q 2600/178; C12Q 2600/158; A61K 38/4813; G01N 2500/00; G01N 2800/2821; A61K 38/1709; A61K 38/45; G01N 2800/52; G01N 2800/2814; G01N 2800/56; C12Q 2600/136; C12Q 1/6883; G01N 2800/50; A61K 38/44; C07K 2317/51; A61K 38/46; G01N 2333/902; G01N 2333/47; G01N 2333/70546; G01N 2333/948; G01N 2333/914; G01N 2333/8121; G01N 2333/9643; G01N 2333/705

(57) ABSTRACT

Methods for treatment and diagnosis of pervasive developmental disorders in humans are described.

### CROSS-REFERENCE TO RELATED APPLICATIONS

This application is a continuation of U.S. patent application Ser. No. 15/493,383, filed Apr. 21, 2017, which is a continuation of U.S. patent application Ser. No. 15/265,174, filed on Sep. 14, 2016, which is a continuation of U.S. patent application Ser. No. 14/383,450, filed on Sep. 5, 2014, which is a 35 U.S.C. § 371 national stage application of Int. Appl. No. PCT/US2013/029201, filed on Mar. 5, 2013, which claims priority to U.S. Provisional Appl. Ser. No. 61/606,935, filed on Mar. 5, 2012. The entire contents of each of the foregoing applications are expressly incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 4, 2017, is named 119992-05906_SeqListing.txt and is 1,144,251 bytes in size.

### BACKGROUND OF THE INVENTION

Pervasive developmental disorders are an important public health concern. This is especially true for autism spectrum disorders such as autism and Asperger's syndrome, which are prevalent, debilitating conditions that begin in early childhood and for which effective treatments are needed. The disorders have a complex etiology that is not well understood.

Autism spectrum disorders are highly heritable, but environmental causes also play an important role. The concordance rate is about 90% for monozygotic twins and about 10% in dizygotic twins. Specific genes associated with autism spectrum disorders have been identified; however, autism spectrum disorder is associated with known genetic predispositions in only about 10-15% of cases (Levy, S. E., et al. Lancet 374(9701): 1627-1638 (2010), hereinafter Levy et al.). Moreover, none of these genetic predispositions are specific to the development of pervasive developmental disorders.

Various neurobiological abnormalities have been observed in autism spectrum disorders. These disorders are characterized by macrocephaly; overgrowth in cortical white matter and abnormal patterns of growth in the frontal lobe, temporal lobes, and limbic structures such as the amygdale; and cytoarchitectural abnormalities in cortical minicolumns and in the cerebellum. Recent findings indicate that the brains of autistic individuals exhibit dysregulation of proteins that are involved in apoptosis and in the normal lamination and maintenance of synaptic plasticity of the brain.

There exists a need in the art for methods of treatment, prevention, reduction, diagnosis and prognosis of pervasive developmental disorders.

### SUMMARY OF THE INVENTION

The present invention is based, at least in part, on the discovery that the proteins listed in Tables 2-6 are modulated, e.g., upregulated or downregulated, in cells derived from a subject afflicted with Autism or Alzheimer's disease, as compared to normal, control cells, e.g., cells derived from a subject that is not afflicted with Autism or Alzheimer's disease (e.g., cells derived from an unaffected sibling or parent of the afflicted subject). Accordingly, the prevent invention provides methods for treating, alleviating symptoms of, inhibiting progression of, preventing, diagnosing, or prognosing a pervasive developmental disorder in a subject involving one or more of the proteins listed in Tables 2-6.

Specifically, in one aspect the invention provides methods of assessing whether a subject is afflicted with a pervasive developmental disorder, the method comprising: (1) determining a level of expression of one or more of the markers listed in Tables 2-6 in a biological sample obtained from the subject, using reagents that transform the markers such that the markers can be detected; (2) comparing the level of expression of the one or more markers in the biological sample obtained from the subject with the level of expression of the one or more markers in a control sample; and (3) assessing whether the subject is afflicted with a pervasive developmental disorder, wherein a modulation in the level of expression of the one or more markers in the biological sample obtained from the subject relative to the level of expression of the one or more markers in the control sample is an indication that the subject is afflicted with a pervasive developmental disorder.

In another aspect, the invention provides methods of prognosing whether a subject is predisposed to developing a pervasive developmental disorder, the method comprising: (1) determining a level of expression of one or more of the markers listed in Tables 2-6 present in a biological sample obtained from the subject, using reagents that transform the markers such that the markers can be detected; (2) comparing the level of expression of the one or more markers present in the biological sample obtained from the subject with the level of expression of the one or more markers present in a control sample; and (3) prognosing whether the subject is predisposed to developing a pervasive developmental disorder, wherein a modulation in the level of expression of the one or more proteins in the biological sample obtained from the subject relative to the level of expression of the one or more proteins in the control sample is an indication that the subject is predisposed to developing a pervasive developmental disorder.

In another aspect, the invention provides methods of prognosing the severity of a pervasive developmental disorder in a subject, the method comprising (1) determining a level of expression of one or more of the markers listed in Tables 2-6 in a biological sample obtained from the subject, using reagents that transform the markers such that the markers can be detected; (2) comparing the level of expression of the one or more markers in the biological sample obtained from the subject with the level of expression of the one or more markers in a control sample; and (3) assessing the severity of the pervasive developmental disorder, wherein a modulation in the level of expression of the one or more markers in the biological sample obtained from the subject relative to the level of expression of the one or more markers in the control sample is an indication of the severity of the pervasive developmental disorder in the subject.

In some embodiments, modulation of the level of expression of the one or more markers in the sample from the subject away from the levels of expression of a control sample by, e.g., at least 2-fold, 3-fold, 4-fold, 5-fold, 10-fold, 15-fold, 10-fold, 30-fold, 40-fold, 50-fold, 100-fold or greater, is an indication that the pervasive developmental disorder in the subject is severe. In some embodiments, modulation of the level of expression of the one or more markers in the sample from the subject further away from levels of expression in a control sample than that of the levels of expression in a sample from a subject suffering from a non-severe form of a pervasive developmental disorder is an indication that the pervasive developmental disorder in the subject is severe.

In some embodiments, modulation of the level of expression of the one or more markers in the sample from the subject towards the levels of expression of a control sample by, e.g., at least 2-fold, 3-fold, 4-fold, 5-fold, 10-fold, 15-fold, 10-fold, 30-fold, 40-fold, 50-fold, 100-fold or greater, is an indication that the pervasive developmental disorder in the subject is not severe. In some embodiments, modulation of the level of expression of the one or more markers in the sample from the subject closer to the levels of expression in a control sample than that of the levels of expression in a sample from a subject suffering from a severe form of a pervasive developmental disorder is an indication that the pervasive developmental disorder in the subject is not severe.

In another aspect, the invention provides methods for monitoring the progression of a pervasive developmental disorder or symptoms of a pervasive developmental disorder in a subject, the method comprising: (1) determining a level of expression of one or more of the markers listed in Tables 2-6 present in a first biological sample obtained from the subject at a first time, using reagents that transform the markers such that the markers can be detected; (2) determining a level of expression of the one or more of the markers listed in Tables 2-6 present in a second biological sample obtained from the subject at a second, later time, using reagents that transform the markers such that the markers can be detected; and (3) comparing the level of expression of the one or more markers listed in Tables 2-6 present in a first sample obtained from the subject at the first time with the level of expression of the one or more markers present in a second sample obtained from the subject at the second, later time; and (4) monitoring the progression of the pervasive developmental disorder, wherein a modulation in the level of expression of the one or more markers in the second sample as compared to the first sample is an indication of the progression of the pervasive developmental disorder or symptoms of the pervasive developmental disorder in the subject.

In one embodiment, modulation of the level of expression in the second sample away from the levels of expression in a control sample, e.g., further away from normal or control levels of expression than that of the levels of expression in the first sample at the first time, is an indication of the progression of the pervasive developmental disorder or symptoms of the pervasive developmental disorder in the subject.

In one embodiment, a lack of modulation in the level of expression in the second sample as compared to the first sample (e.g., the levels of expression in the first and second sample are approximately the same) is an indication that the pervasive developmental disorder or symptoms of the pervasive developmental disorder have not progressed in the subject. In one embodiment, modulation of the level of expression in the second sample towards the levels of expression in a control sample, e.g., closer to normal or control levels of expression than that of the levels of expression in the first sample at the first time, is an indication that the pervasive developmental disorder or symptoms of the pervasive developmental disorder have not progressed in the subject.

In one embodiment, the methods further comprise selecting a treatment regimen for the subject identified as being afflicted with a pervasive developmental disorder or predisposed to developing a pervasive developmental disorder.

In one embodiment, the method further comprise administering a treatment regimen to the subject identified as being afflicted with a pervasive developmental disorder or predisposed to developing a pervasive developmental disorder.

In one embodiment, the method further comprise continuing administration of an ongoing treatment regimen to the subject for whom the progression of the pervasive developmental disorder is determined to be reduced, delayed or lessened.

In another aspect, the invention provides a method for assessing the efficacy of a treatment regimen for treating a pervasive developmental disorder or symptoms of a pervasive developmental disorder in a subject, the method comprising:

(1) determining a level of expression of one or more of the markers listed in Tables 2-6 present in a first biological sample obtained from the subject prior to administering at least a portion of the treatment regimen to the subject, using reagents that transform the markers such that the markers can be detected;

(2) determining a level of expression of one or more of the markers listed in Tables 2-6 present in a second biological sample obtained from the subject following administration of at least a portion of the treatment regimen to the subject, using reagents that transform the markers such that the markers can be detected;

(3) comparing the level of expression of one or more markers listed in Tables 2-6 present in a first sample obtained from the subject prior to administering at least a portion of the treatment regimen to the subject with the level of expression of the one or more markers present in a second sample obtained from the subject following administration of at least a portion of the treatment regimen; and

(4) assessing whether the treatment regimen is efficacious for treating the pervasive developmental disorder or symptoms of the pervasive developmental disorder, wherein a modulation in the level of expression of the one or more markers in the second sample as compared to the first sample is an indication that the treatment regimen is efficacious for treating the pervasive developmental disorder or symptoms of the pervasive developmental disorder in the subject.

In one embodiment, the method further comprises continuing administration of the treatment regimen to the subject for whom the treatment regimen is determined to be efficacious for treating the pervasive developmental disorder or symptoms of the pervasive developmental disorder, or discontinuing administration of the treatment regimen to the subject for whom the treatment regimen is determined to be non-efficacious for treating the pervasive developmental disorder or symptoms of the pervasive developmental disorder.

In another aspect, the invention provides a method of identifying a compound for treating a pervasive developmental disorder or symptoms of pervasive developmental disorders in a subject, the method comprising:

(1) contacting a biological sample with a test compound;

(2) determining the level of expression of one or more markers listed in Tables 2-6 present in the biological sample;

(3) comparing the level of expression of the one or more markers in the biological sample with that of a control sample not contacted by the test compound; and

(4) selecting a test compound that modulates the level of expression of the one or more markers in the biological sample,

thereby identifying a compound for treating a pervasive developmental disorder or symptoms of a pervasive developmental disorder in a subject.

In one embodiment, the pervasive developmental disorder is an autism spectrum disorder.

In one embodiment, the pervasive developmental disorder is autistic disorder.

In one embodiment, the pervasive developmental disorder is Alzheimer's disease.

In one embodiment, the pervasive developmental disorder is autism and Alzheimer's disease. In one embodiment, the pervasive developmental disorder is autism and alzheimer's disease, and the markers are one or more of the markers listed in Table 3.

In one embodiment, the pervasive developmental disorder is Asperger's syndrome.

In one embodiment, the pervasive developmental disorder is pervasive developmental disorder-not otherwise specified.

In one embodiment, the subject suffers from a pervasive developmental disorder.

In one embodiment, the subject exhibits subsyndromal manifestations of a pervasive developmental disorder.

In one embodiment, the subject is suspected to suffer from or be predisposed to developing a pervasive developmental disorder.

In one embodiment, the sample obtained from the subject is processed such that the sample is transformed, thereby allowing the determination of a level of expression of one or more of the markers listed in Tables 2-6.

In one embodiment, the level of expression of the one or more markers is determined at a nucleic acid level.

In one embodiment, the level of expression of the one or more markers is determined by detecting RNA. In one embodiment, the level of expression of the one or more markers is determined by detecting mRNA, miRNA, or hnRNA. In one embodiment, the level of expression of the one or more markers is determined by detecting DNA. In one embodiment, the level of expression of the one or more markers is determined by detecting cDNA.

In one embodiment, the level of expression of the one or more markers is determined by using a technique selected from the group consisting of a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and a combination or sub-combination thereof.

In one embodiment, determining the level of expression of the one or more markers comprises performing an immunoassay using an antibody.

In one embodiment, the one or more markers comprises a protein.

In one embodiment, the protein is detected using a binding protein that binds at least one of the one or more markers.

In one embodiment, the binding protein comprises an antibody, or antigen binding fragment thereof, that specifically binds to the protein.

In one embodiment, the antibody or antigen binding fragment thereof is selected from the group consisting of a murine antibody, a human antibody, a humanized antibody, a bispecific antibody, a chimeric antibody, a Fab, Fab′, F(ab′)₂, scFv, SMIP, affibody, avimer, versabody, nanobody, a domain antibody, and an antigen binding fragment of any of the foregoing.

In one embodiment, the binding protein comprises a multispecific binding protein.

In one embodiment, the multispecific binding protein comprises a dual variable domain immunoglobulin (DVD-IgTM) molecule, a halfhalf-body DVD-Ig (hDVD-Ig) molecule, a triple variable domain immunoglobulin (TVD-IgtDVD-Ig) molecule, and a receptor variable domain immunoglobulin (rDVD-Ig) molecule. In one example, the multispecific binding protein (e.g., a polyvalent DVD-Ig (pDVD-Ig) molecule), a monobody DVD-Ig (mDVD-Ig) molecule, a cross over (coDVD-Ig) molecule, a blood brain barrier (bbbDVD-Ig) molecule, a cleavable linker DVD-Ig (clDVD-Ig) molecule, or a redirected cytotoxicity DVD-Ig (rcDVD-Ig) molecule.

In one embodiment, the antibody or antigen binding fragment thereof comprises a label.

In one embodiment, the label is selected from the group consisting of a radio-label, a biotin-label, a chromophore, a fluorophore, and an enzyme.

In one embodiment, the level of expression of at least one of the one or more markers is determined by using a technique selected from the group consisting of an immunoassay, a western blot analysis, a radioimmunoassay, immunofluorimetry, immunoprecipitation, equilibrium dialysis, immunodiffusion, an electrochemiluminescence immunoassay (ECLIA), an ELISA assay, a polymerase chain reaction, an immunopolymerase chain reaction, and combinations or sub-combinations thereof.

In one embodiment, the immunoassay comprises a solution-based immunoassay selected from the group consisting of electrochemiluminescence, chemiluminescence, fluorogenic chemiluminescence, fluorescence polarization, and time-resolved fluorescence.

In one embodiment, the immunoassay comprises a sandwich immunoassay selected from the group consisting of electrochemiluminescence, chemiluminescence, and fluorogenic chemiluminescence.

In one embodiment, the sample comprises a fluid, or component thereof, obtained from the subject. In one embodiment, the fluid is selected from the group consisting of blood, serum, synovial fluid, lymph, plasma, urine, amniotic fluid, aqueous humor, vitreous humor, bile, breast milk, cerebrospinal fluid, cerumen, chyle, cystic fluid, endolymph, feces, gastric acid, gastric juice, mucus, nipple aspirates, pericardial fluid, perilymph, peritoneal fluid, pleural fluid, pus, saliva, sebum, semen, sweat, serum, sputum, tears, vaginal secretions, and fluid collected from a biopsy.

In one embodiment, the sample comprises a tissue or cell, or component thereof, obtained from the subject.

In another aspect, the invention provides a method for treating, alleviating symptoms of, inhibiting progression of, or preventing a pervasive developmental disorder in a subject, the method comprising administering to the subject in need thereof a therapeutically effective amount of a pharmaceutical composition comprising one or more of the markers listed in Tables 2-6.

In another aspect, the invention provides a method for treating, alleviating symptoms of, inhibiting progression of, or preventing a pervasive developmental disorder in a subject, the method comprising administering to the subject in need thereof a therapeutically effective amount of a pharmaceutical composition comprising an agent that modulates expression or activity of one or more of the markers listed in Tables 2-6.

In one embodiment, the agent inhibits expression or activity of one or more of the markers listed in Tables 2-6.

In one embodiment, the agent augments expression or activity of one or more of the markers listed in Tables 2-6.

In another aspect, the invention provides a method of identifying an agent that modulates the expression or activity of one or more of the markers listed in Tables 2-6, comprising contacting the one or more markers with a test agent, detecting the expression or activity of the one or more markers contacted with the test agent, comparing the expression or activity of the one or more markers contacted with the test agent with the activity of a control, e.g., expression or activity of the one or more markers not contacted with the test agent, and identifying an agent that modulates the expression or activity of the one or more markers.

In one embodiment, the agent down-modulates at least one of the one or more markers listed in Tables 2-6.

In one embodiment, the agent up-modulates at least one of the one or more markers listed in Tables 2-6.

In another aspect, the invention provides a method for treating, alleviating symptoms of, inhibiting progression of, or preventing a pervasive developmental disorder in a subject, the method comprising administering to the subject in need thereof a therapeutically effective amount of a pharmaceutical composition comprising an agent identified according to the foregoing methods.

In one embodiment of all of the foregoing aspects, the subject is a human subject.

The invention described herein is based, at least in part, on a novel, collaborative utilization of network biology, genomic, proteomic, metabolomic, transcriptomic, and bioinformatics tools and methodologies, which, when combined, may be used to study selected disease conditions including pervasive developmental disorder, such as autism and Alzheimer's disease, using a systems biology approach. In a first step of the Platform Technology, cellular modeling systems are developed to probe the disease process, e.g., pervasive development disorder, including autism, comprising disease-related cells, optionally subjected to various disease-relevant environment stimuli (e.g., hyperglycemia, hypoxia, immuno-stress, and lipid peroxidation). In some embodiments, the cellular modeling system involves cellular cross-talk mechanisms between various interacting cell types. In a second step, high throughput biological readouts from the cell model system are obtained by using a combination of techniques, including, for example, mass spectrometry (LC/MSMS), flow cytometry, cell-based assays, and functional assays. In a third step, the high throughput biological readouts are then subjected to a bioinformatic analysis to study congruent data trends by in vitro, in vivo, and in silico modeling. The resulting matrices allow for cross-related data mining where linear and non-linear regression analysis are carried out to identify conclusive pressure points (or “hubs”). These “hubs”, as presented herein, are candidates for drug discovery. In particular, these hubs represent potential drug targets and/or biological markers for pervasive developmental disorders.

The molecular signatures of the differentials between the disease (e.g., pervasive developmental disorder) and normal phenotype allow for insight into the mechanisms that lead to disease onset and progression. Taken together, the combination of the Platform Technology described above with strategic cellular modeling allows for robust intelligence that can be employed to further our understanding of the disease while simultaneously creating biomarker libraries and drug candidates that may clinically augment standard of care.

A significant feature of the platform of the invention is that the AI-based system is based on the data sets obtained from the cell model system, without resorting to or taking into consideration any existing knowledge in the art, such as known biological relationships (i.e., no data points are artificial), concerning the biological process. Accordingly, the resulting statistical models generated from the platform are unbiased. Another significant feature of the platform of the invention and its components, e.g., the cell model systems and data sets obtained therefrom, is that it allows for continual building on the cell models over time (e.g., by the introduction of new cells and/or conditions), such that an initial, “first generation” consensus causal relationship network generated from a cell model for a pervasive developmental disorder, e.g., autism, can evolve along with the evolution of the cell model itself to a multiple generation causal relationship network (and delta or delta-delta networks obtained therefrom). In this way, both the cell models, the data sets from the cell models, and the causal relationship networks generated from the cell models by using the Platform Technology methods can constantly evolve and build upon previous knowledge obtained from the Platform Technology.

Accordingly, in one aspect, the invention provides a method for identifying a modulator of a disease process, e.g., pervasive developmental disorder, said method comprising: (1) establishing a disease model for the disease process, e.g., pervasive developmental disorder, using disease related cells, e.g. cells related to a pervasive developmental disorder, to represent a characteristic aspect of the disease process, e.g., pervasive developmental disorder; (2) obtaining a first data set from the disease model, wherein the first data set represents expression levels of a plurality of genes in the disease related cells; (3) optionally, obtaining a second data set from the disease model, wherein the second data set represents a functional activity or a cellular response of the disease related cells; (4) generating a consensus causal relationship network among the expression levels of the plurality of genes and/or the functional activity or cellular response based solely on the first data set and optionally the second data set using a programmed computing device, wherein the generation of the consensus causal relationship network is not based on any known biological relationships other than the first data set and the second data set; (5) identifying, from the consensus causal relationship network, a causal relationship unique in the disease process (e.g., pervasive developmental disorder), wherein a gene associated with the unique causal relationship is identified as a modulator of the disease process (e.g., pervasive developmental disorder).

In certain embodiments, the disease process is pervasive developmental disorder.

In certain embodiments, the disease process is autism or autism spectrum disorder.

In certain embodiments, the modulator stimulates or promotes the disease process.

In certain embodiments, the modulator inhibits the disease process.

In certain embodiments, the modulator shifts the energy metabolic pathway specifically in disease cells from a glycolytic pathway towards an oxidative phosphorylation pathway.

In certain embodiments, the disease model comprises an in vitro culture of disease cells, optionally further comprising a matching in vitro culture of control or normal cells.

In certain embodiments, the in vitro culture of the disease cells is subject to an environmental perturbation, and the in vitro culture of the matching control cells is identical disease cells not subject to the environmental perturbation.

In certain embodiments, the environmental perturbation comprises one or more of a contact with an agent, a change in culture condition, an introduced genetic modification/mutation, and a vehicle (e.g., vector) that causes a genetic modification/mutation.

In certain embodiments, the first data set comprises protein and/or mRNA expression levels of the plurality of genes.

In certain embodiments, the first data set further comprises one or more of lipidomics data, metabolomics data, transcriptomics data, and single nucleotide polymorphism (SNP) data.

In certain embodiments, the second data set comprises one or more of bioenergetics profiling, cell proliferation, apoptosis, organellar function, and a genotype-phenotype association actualized by functional models selected from ATP, ROS, OXPHOS, and Seahorse assays.

In certain embodiments, step (4) is carried out by an artificial intelligence (AI)-based informatics platform.

In certain embodiments, the AI-based informatics platform comprises REFS™.

In certain embodiments, the AI-based informatics platform receives all data input from the first data set and the second data set without applying a statistical cut-off point.

In certain embodiments, the consensus causal relationship network established in step (4) is further refined to a simulation causal relationship network, before step (5), by in silico simulation based on input data, to provide a confidence level of prediction for one or more causal relationships within the consensus causal relationship network.

In certain embodiments, the unique causal relationship is identified as part of a differential causal relationship network that is uniquely present in disease cells, and absent in the matching control cells.

In certain embodiments, the method further comprises validating the identified unique causal relationship in a biological system.

In another aspect, the invention relates to a method for providing a disease model for pervasive developmental disorder for use in a platform method, comprising: establishing a disease model for a pervasive developmental disorder, using disease related cells, e.g., cells related to a pervasive developmental disorder, to represent a characteristic aspect of the pervasive developmental disorder, wherein the disease model for pervasive developmental disorder is useful for generating disease model data sets used in the platform method; thereby providing a disease model for pervasive developmental disorder for use in a platform method.

In another aspect, the invention relates to a method for obtaining a first data set and second data set from a disease model for pervasive developmental disorder for use in a platform method, comprising: (1) obtaining a first data set from a disease model for pervasive developmental disorder for use in a platform method, wherein the disease model comprises disease related cells, e.g., cells related to a pervasive developmental disorder, and wherein the first data set represents expression levels of a plurality of genes in the disease related cells; (2) optionally obtaining a second data set from the disease model for use in a platform method, wherein the second data set represents a functional activity or a cellular response of the disease related cells; thereby obtaining a first data set and second data set from the disease model for pervasive developmental disorder; thereby obtaining a first data set and second data set from a disease model for pervasive developmental disorder for use in a platform method.

In another aspect, the invention relates to a method for identifying a modulator of a pervasive developmental disorder, said method comprising: (1) generating a consensus causal relationship network among a first data set and optionally a second data set obtained from a disease model for a pervasive developmental disorder, wherein the disease model for a pervasive developmental disorder comprises disease cells, e.g. cells related to a pervasive developmental disorder, and wherein the first data set represents expression levels of a plurality of genes in the disease related cells and the second data set represents a functional activity or a cellular response of the disease related cells, using a programmed computing device, wherein the generation of the consensus causal relationship network is not based on any known biological relationships other than the first data set and the second data set; (2) identifying, from the consensus causal relationship network, a causal relationship unique in the pervasive developmental disorder, wherein a gene associated with the unique causal relationship is identified as a modulator of a pervasive developmental disorder; thereby identifying a modulator of a pervasive developmental disorder.

In another aspect, the invention relates to a method for identifying a modulator of a pervasive developmental disorder, said method comprising: 1) providing a consensus causal relationship network generated from a disease model for the pervasive developmental disorder; 2) identifying, from the consensus causal relationship network, a causal relationship unique in the pervasive developmental disorder, wherein a gene associated with the unique causal relationship is identified as a modulator of a pervasive developmental disorder; thereby identifying a modulator of a pervasive developmental disorder.

In certain embodiments, the consensus causal relationship network is generated among a first data set and second data set obtained from the disease model for the pervasive developmental disorder, wherein the disease model comprises disease cells, e.g., cells related to a pervasive developmental disorder, and wherein the first data set represents expression levels of a plurality of genes in the disease related cells and the second data set represents a functional activity or a cellular response of the disease related cells, using a programmed computing device, wherein the generation of the consensus causal relationship network is not based on any known biological relationships other than the first data set and the second data set.

In certain embodiments, the disease process is pervasive developmental disorder.

In certain embodiments, the disease process is autism or autism spectrum disorder.

In certain embodiments, the modulator stimulates or promotes the disease process.

In certain embodiments, the modulator inhibits the disease process.

In certain embodiments, the modulator shifts the energy metabolic pathway specifically in disease cells from a glycolytic pathway towards an oxidative phosphorylation pathway.

In certain embodiments, the disease model comprises an in vitro culture of disease cells, optionally further comprising a matching in vitro culture of control or normal cells.

In certain embodiments, the in vitro culture of the disease cells is subject to an environmental perturbation, and the in vitro culture of the matching control cells is identical disease cells not subject to the environmental perturbation.

In certain embodiments, the environmental perturbation comprises one or more of a contact with an agent, a change in culture condition, an introduced genetic modification/mutation, and a vehicle (e.g., vector) that causes a genetic modification/mutation.

In certain embodiments, the first data set comprises protein and/or mRNA expression levels of the plurality of genes.

In certain embodiments, the first data set further comprises one or more of lipidomics data, metabolomics data, transcriptomics data, and single nucleotide polymorphism (SNP) data.

In certain embodiments, the second data set comprises one or more of bioenergetics profiling, cell proliferation, apoptosis, organellar function, and a genotype-phenotype association actualized by functional models selected from ATP, ROS, OXPHOS, and Seahorse assays.

In certain embodiments, step (4) is carried out by an artificial intelligence (AI)-based informatics platform.

In certain embodiments, the AI-based informatics platform comprises REFS™.

In certain embodiments, the AI-based informatics platform receives all data input from the first data set and the second data set without applying a statistical cut-off point.

In certain embodiments, the consensus causal relationship network established in step (4) is further refined to a simulation causal relationship network, before step (5), by in silico simulation based on input data, to provide a confidence level of prediction for one or more causal relationships within the consensus causal relationship network.

In certain embodiments, the unique causal relationship is identified as part of a differential causal relationship network that is uniquely present in disease cells, and absent in the matching control cells.

In certain embodiments, the method further comprising validating the identified unique causal relationship in a biological system.

In certain embodiments, the “environmental perturbation”, also referred to herein as “external stimulus component”, is a therapeutic agent. In certain embodiments, the external stimulus component is a small molecule (e.g., a small molecule of no more than 5 kDa, 4 kDa, 3 kDa, 2 kDa, 1 kDa, 500 Dalton, or 250 Dalton). In certain embodiments, the external stimulus component is a biologic. In certain embodiments, the external stimulus component is a chemical. In certain embodiments, the external stimulus component is endogenous or exogenous to cells. In certain embodiments, the external stimulus component is a MIM or epishifter. In certain embodiments, the external stimulus component is a stress factor for the cell system, such as hypoxia, hyperglycemia, hyperlipidemia, hyperinsulinemia, and/or lactic acid rich conditions.

In certain embodiments, the external stimulus component may include a therapeutic agent or a candidate therapeutic agent for treating a disease condition, including chemotherapeutic agent, protein-based biological drugs, antibodies, fusion proteins, small molecule drugs, lipids, polysaccharides, nucleic acids, etc.

In certain embodiments, the external stimulus component may be one or more stress factors, such as those typically encountered in vivo under the various disease conditions, including hypoxia, hyperglycemic conditions, acidic environment (that may be mimicked by lactic acid treatment), etc.

In other embodiments, the external stimulus component may include one or more MIMs and/or epishifters, as defined herein below. MIMs and epishifters are further described in U.S. application Ser. Nos. 12/777,902, 12/778,029, 12/778,054, and 12/778,010, the entire contents of which are hereby expressly incorporated herein by reference. Exemplary MIMs include Coenzyme Q10 (also referred to herein as CoQ10), compounds in the Vitamin B family, or nucleosides, mononucleotides or dinucleotides that comprise a compound in the Vitamin B family, vitamin D2, vitamin D3, 1,25-(OH)₂-vitamin D2 and 1,25-(OH)₂-vitamin D3.

In making cellular output measurements (such as protein expression), either absolute amount (e.g., expression amount) or relative level (e.g., relative expression level) may be used. In one embodiment, absolute amounts (e.g., expression amounts) are used. In one embodiment, relative levels or amounts (e.g., relative expression levels) are used. For example, to determine the relative protein expression level of a cell system, the amount of any given protein in the cell system, with or without the external stimulus to the cell system, may be compared to a suitable control cell line or mixture of cell lines (such as all cells used in the same experiment) and given a fold-increase or fold-decrease value. The skilled person will appreciate that absolute amounts or relative amounts can be employed in any cellular output measurement, such as gene and/or RNA transcription level, level of lipid, or any functional output, e.g., level of apoptosis, level of toxicity, or ECAR or OCR as described herein. A pre-determined threshold level for a fold-increase (e.g., at least 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 75 or 100 or more fold increase) or fold-decrease (e.g., at least a decrease to 0.9, 0.8, 0.75, 0.7, 0.6, 0.5, 0.45, 0.4, 0.35, 0.3, 0.25, 0.2, 0.15, 0.1 or 0.05 fold, or a decrease to 90%, 85%, 80%, 75%, 70%, 65%, 60%, 55%, 50%, 45%, 40%, 35%, 30%, 25%, 20%, 15%, 10% or 5% or less) may be used to select significant differentials, and the cellular output data for the significant differentials may then be included in the data sets (e.g., first and second data sets) utilized in the platform technology methods of the invention. The skilled person will recognize that all values presented in the foregoing list can also be the upper or lower limit of ranges, e.g., between 1.5 and 5 fold, 5 and 10 fold, 2 and 5 fold, or between 0.9 and 0.7, 0.9 and 0.5, or 0.7 and 0.3 fold, which are intended to be a part of this invention.

Throughout the present application, all values presented in a list, e.g., such as those above, can also be the upper or lower limit of ranges that are intended to be a part of this invention.

In one embodiment of the methods of the invention, not every observed causal relationship in a causal relationship network may be of biological significance. With respect to any given biological system for which the subject interrogative biological assessment is applied, some (or maybe all) of the causal relationships (and the genes associated therewith) may be “determinative” with respect to the specific biological problem at issue, e.g., either responsible for causing a disease condition (a potential target for therapeutic intervention) or is a biomarker for the disease condition (a potential diagnostic or prognostic factor). In one embodiment, an observed causal relationship unique in the biological system is determinative with respect to the specific biological problem at issue. In one embodiment, not every observed causal relationship unique in the biological system is determinative with respect to the specific problem at issue.

Such determinative causal relationships may be selected by an end user of the subject method, or it may be selected by a bioinformatics software program, such as REFS, DAVID-enabled comparative pathway analysis program, or the KEGG pathway analysis program. In certain embodiments, more than one bioinformatics software program is used, and consensus results from two or more bioinformatics software programs are preferred.

As used herein, “differentials” of cellular outputs include differences (e.g., increased or decreased levels) in any one or more parameters of the cellular outputs. In certain embodiments, the differentials are each independently selected from the group consisting of differentials in mRNA transcription, protein expression, protein activity, metabolite/intermediate level, and/or ligand-target interaction. For example, in terms of protein expression level, differentials between two cellular outputs, such as the outputs associated with a cell system before and after the treatment by an external stimulus component, can be measured and quantitated by using art-recognized technologies, such as mass-spectrometry based assays (e.g., iTRAQ, 2D-LC-MSMS, etc.).

### BRIEF DESCRIPTION OF THE DRAWINGS

FIG. 1: Illustration of the “Omics” Cascades.

FIG. 2: Illustration of the Interrogative Biology® Platform.

FIG. 3: Illustration of the Interrogative Biology® Platform.

FIG. 4A-4D: High level schematic illustration of the components and process for an AI-based informatics system that may be used with exemplary embodiments.

FIG. 5: Flow chart of process in AI-based informatics system that may be used with some exemplary embodiments.

FIG. 6: Schematic depicting an exemplary computing environment suitable for practicing exemplary embodiments taught herein.

FIG. 7: High level flow chart of an exemplary method, in accordance with some embodiments.

FIG. 8: Illustration of the experimental approach for identification of novel biomarkers of autism.

FIG. 9: Illustration of source of experimental samples for identification of novel biomarkers of autism.

FIG. 10: A global differential network with hubs/nodes unique in autism versus normal samples.

FIG. 11: A network of molecular entities driven by “disease state” common to Autism and Alzheimer's Disease.

FIG. 12: An exemplary causal molecular interaction network in autism.

FIG. 13: An exemplary sub-network with SPTAN1 as a critical hub in autism interaction network.

FIG. 14: An exemplary sub-network with GLUD1 as a critical hub in autism interaction network.

FIG. 15: An exemplary sub-network with CORO1A as a critical hub in autism interaction network.

### DETAILED DESCRIPTION OF THE INVENTION

Autism Spectrum Disorders (ASD) is a pervasive developmental disorder including a group of serious and enigmatic neuro-behavioral disorders. Autism is a complex neurodevelopmental disorder. The major characteristics of this disease are the impairment in social skills, difficulty to communicate, and restricted/repetitive behaviors. Currently, it is the third most common developmental disorder. The number of children diagnosed with autism has dramatically increased and now considered epidemic with current incidence of 1 in 110 children with a 4:1 male-female ratio. Although Autism does not affect the patient life-span, it could be a lifelong disorder. ASD has many suspected causes, including genetic mutations and/or deletions, mitochondria dysfunction, immunologic, diet, mercury poisoning and viral infections. Interesting, mitochondrial dysfunction has been shown to play a crucial role in the disease pathophysiology. As a multi-factorial disease, autism has a very diverse patient population under one spectrum. Due to the poor understanding of underlying molecular mechanisms of the disease, the current diagnosis is based on observational behavior variables, with no drug approved to treat autism specifically. Currently, there are no established molecular signatures or end-points used in the clinical environment for diagnosis. No biological markers have been validated to reliably diagnose autism in an individual patient. Therefore, the absence of biological markers for ASD is a major bottleneck to arbitrating diagnosis, and for developing drugs for the treatment and/or prevention of the disorder.

In the past, a significant effort has been placed on Autism genomics/genetics studies. To date, however, no validated biomarkers are available, no objective clinical test can be performed to help the clinicians, and there are no promising treatment to help autistic children and their families. It is possible that this lack of progress is due to the fact that when solely genetic/genomics studies are performed, a global understanding of the molecular mechanism underlying this disease is lost. It is possible that one needs to look at the differential molecular changes at all omic levels (e.g., genomic, proteomic, etc.), including the interactome, to gain a comprehensive understanding of the system of biology behind the autistic phenotypes.

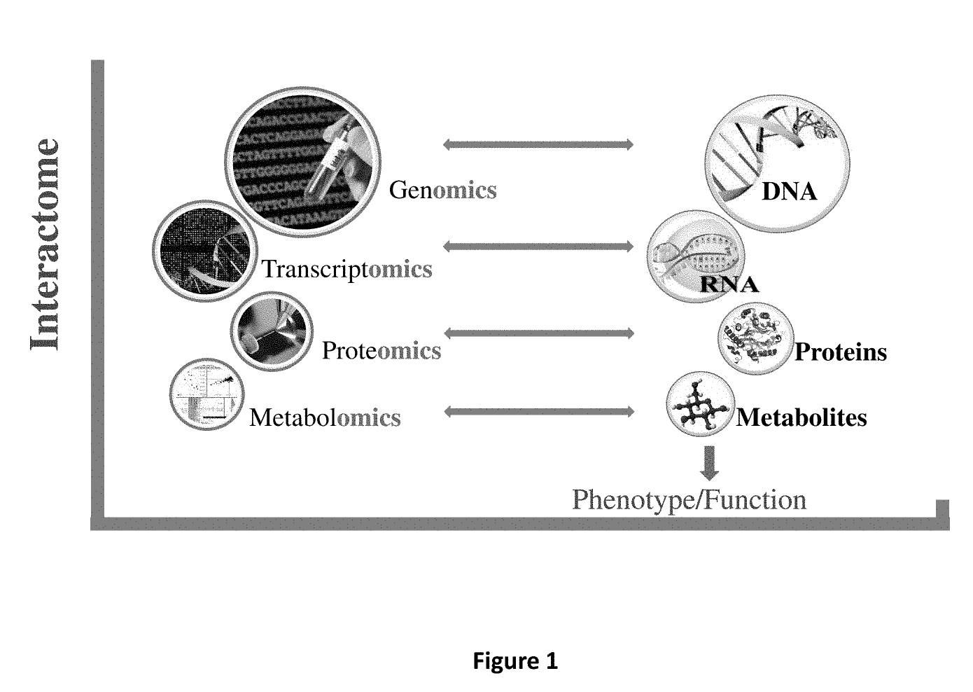

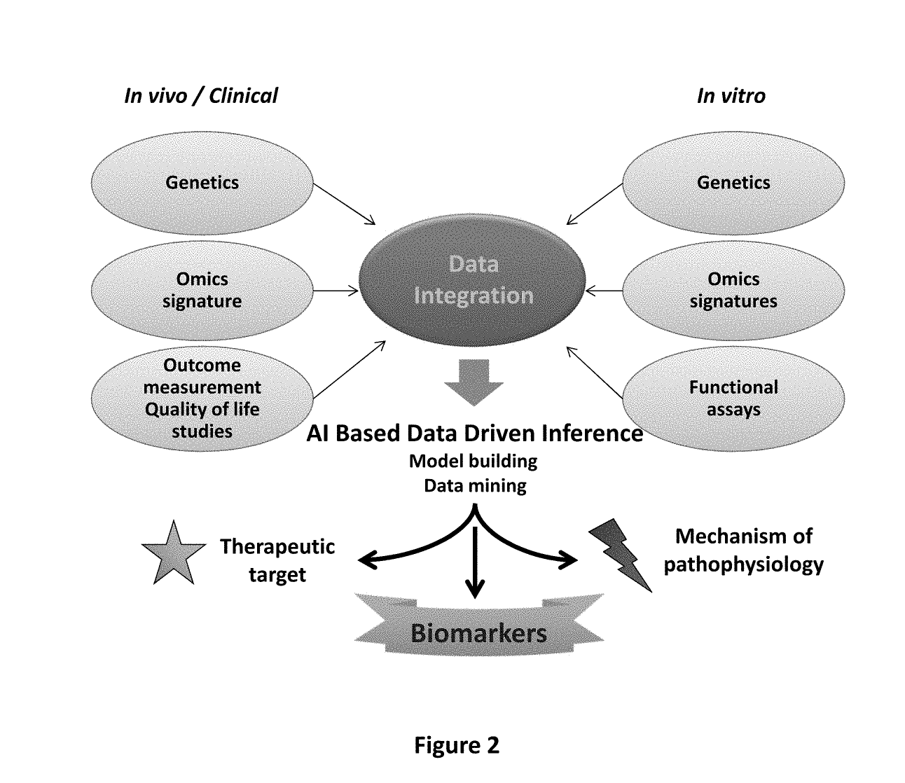

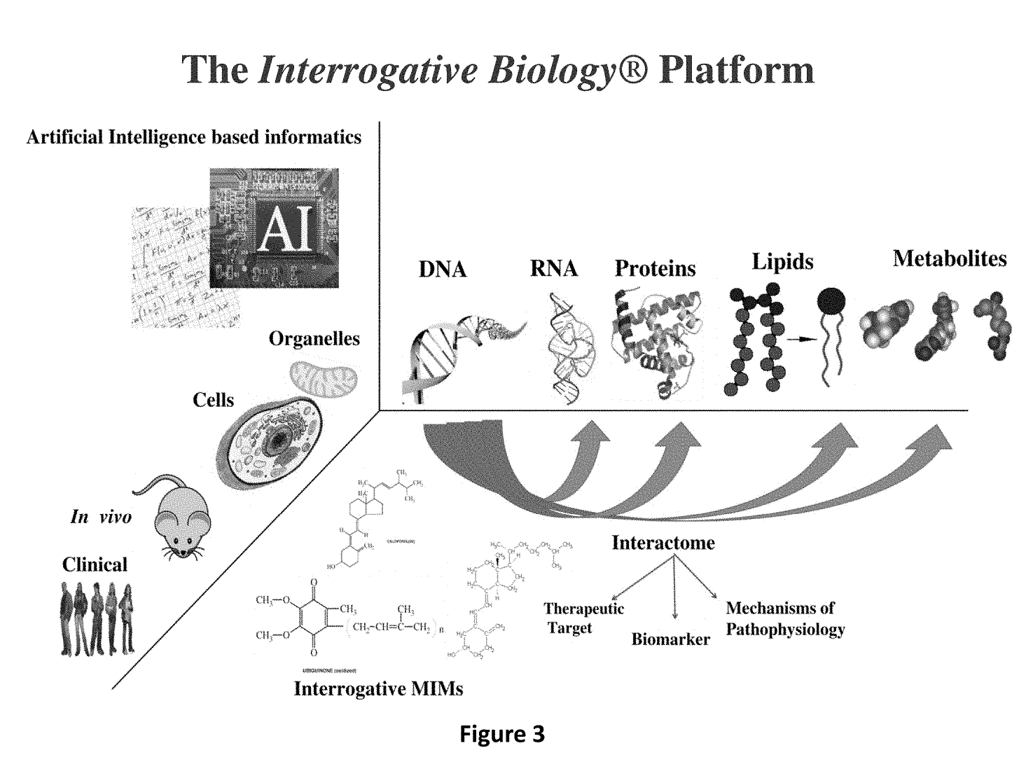

Accordingly, Applicants describe and employ herein a novel approach combining the power of cell biology and multi-omics platforms in an Interrogative Discovery Platform Technology. The Interrogative Platform Technology integrates the data from in vitro and/or in vivo/clinical studies using artificial intelligence (AI) based on data-driven inference in order to mine the data and build bio-models. A schematic depicting the different “Omits” cascades employed in the Platform Technology is provided in FIG. 1. Schematics of the Interrogative Discovery Platform Technology are provided in FIGS. 2-3. This Interrogative Platform Technology is further described in application No. PCT/US2012/027615, the entire contents of which are expressly incorporated herein by reference. Applying the Platform Technology to a cell model system for pervasive developmental disorders has provided insight into the mechanism of pathophysiology of pervasive developmental disorders, and has generated candidate biomarkers as well as potential therapeutic targets and/or therapies/drugs. Candidate drugs/drug targets identified by using this Platform Technology naturally exist in the human body and, therefore, avoid the toxic effects of exogenous therapeutic agents.

### I. Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

As used herein, the term “subject” or “patient” refers to either human and non-human animals, e.g., veterinary patients, preferably a mammal. The term “non-human animal” includes vertebrates, e.g., mammals, such as non-human primates, mice, rodents, rabbits, sheep, dogs, cats, horses, cows, ovine, canine, feline, equine or bovine species. In an embodiment, the subject is a human (e.g., a human with a pervasive developmental disorder). It should be noted that clinical observations described herein were made with human subjects and, in at least some embodiments, the subjects are human.

“Therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

By “patient” is meant any animal (e.g., a human or a non-human mammal), including horses, dogs, cats, pigs, goats, rabbits, hamsters, monkeys, guinea pigs, rats, mice, lizards, snakes, sheep, cattle, fish, and birds.

The terms “marker” or “biomarker” are used interchangeably herein to mean a substance that is used as an indicator of a biologic state, e.g., genes, messenger RNAs (mRNAs, microRNAs (miRNAs); heterogeneous nuclear RNAs (hnRNAs), and proteins, or portions thereof.

The “level of expression” or “expression pattern” refers to a quantitative or qualitative summary of the expression of one or more markers or biomarkers in a subject, such as in comparison to a standard or a control.

A “higher level of expression”, “higher level of activity”, “increased level of expression” or “increased level of activity” refers to an expression level and/or activity in a test sample that is greater than the standard error of the assay employed to assess expression and/or activity, and is preferably at least twice, and more preferably three, four, five or ten or more times the expression level and/or activity of the marker in a control sample (e.g., a sample from a healthy subject not afflicted with a pervasive developmental disorder) and preferably, the average expression level and/or activity of the marker in several control samples.

A “lower level of expression”, “lower level of activity”, “decreased level of expression” or “decreased level of activity” refers to an expression level and/or activity in a test sample that is greater than the standard error of the assay employed to assess expression and/or activity, but is preferably at least twice, and more preferably three, four, five or ten or more times less than the expression level of the marker in a control sample (e.g., a sample that has been calibrated directly or indirectly against a panel of pervasive developmental disorders with follow-up information which serve as a validation standard for prognostic ability of the marker) and preferably, the average expression level and/or activity of the marker in several control samples.

As used herein, “antibody” includes, by way of example, naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

Reference to a gene encompasses naturally occurring or endogenous versions of the gene, including wild type, polymorphic or allelic variants or mutants (e.g., germline mutation, somatic mutation) of the gene, which can be found in a subject. In an embodiment, the sequence of the biomarker gene is at least about 80%, at least about 85%, at least about 90%, at least about 91%, at least about 92%, at least about 93%, at least about 94%, at least about 95%, at least about 96%, at least about 97%, at least about 98%, or at least about 99% identical to the sequence of a marker listed in Tables 2-6. Sequence identity can be determined, e.g., by comparing sequences using NCBI BLAST (e.g., Megablast with default parameters).

In an embodiment, the level of expression of one or more of the markers is determined relative to a control sample, such as the level of expression of the marker in normal tissue (e.g., a range determined from the levels of expression of the marker observed in normal tissue samples). In an embodiment, the level of expression of the marker is determined relative to a control sample, such as the level of expression of the marker in samples from healthy parents or siblings of a diseased subject, or the level of expression of the marker in samples from other healthy subjects. In another embodiment, the level of expression of the one or more markers is determined relative to a control sample, such as the level of expression of the one or more markers in samples from other subjects suffering from a pervasive developmental disorder. For example, the level of expression of one or more markers in Tables 2-6 in samples from other subjects can be determined to define levels of expression that correlate with sensitivity to a particular treatment, and the level of expression of the one or more markers in the sample from the subject of interest is compared to these levels of expression.

The term “known standard level” or “control level” refers to an accepted or pre-determined expression level of one or more markers, for example, one or more markers listed in Tables 2-6, which is used to compare the expression level of the one or more markers in a sample derived from a subject. In one embodiment, the control expression level of the marker is the average expression level of the marker in samples derived from a population of subjects, e.g., the average expression level of the marker in a population of subjects with a pervasive developmental disorder. In another embodiment, the population comprises a group of subjects who do not respond to a particular treatment, or a group of subjects who express the respective marker at high or normal levels. In another embodiment, the control level constitutes a range of expression of the marker in normal tissue. In another embodiment, the control level constitutes a range of expression of the marker in cells or plasma from a variety of subjects having a pervasive developmental disorder. In another embodiment, “control level” refers also to a pre-treatment level in a subject.

As further information becomes available as a result of routine performance of the methods described herein, population-average values for “control” level of expression of the markers of the present invention may be used. In other embodiments, the “control” level of expression of the markers may be determined by determining the expression level of the respective marker in a subject sample obtained from a subject before the suspected onset of a pervasive developmental disorder in the subject, from archived subject samples, from healthy parents or siblings of a diseased subject, and the like.

Control levels of expression of markers of the invention may be available from publicly available databases. In addition, Universal Reference Total RNA (Clontech Laboratories) and Universal Human Reference RNA (Stratagene) and the like can be used as controls. For example, qPCR can be used to determine the level of expression of a marker, and an increase in the number of cycles needed to detect expression of a marker in a sample from a subject, relative to the number of cycles needed for detection using such a control, is indicative of a low level of expression of the marker.

The term “sample” refers to cells, tissues or fluids obtained or isolated from a subject, as well as cells, tissues or fluids present within a subject. The term “sample” includes any body fluid, tissue or a cell or collection of cells from a subject, as well as any component thereof, such as a fraction or an extract. In one embodiment, the tissue or cell is removed from the subject. In another embodiment, the tissue or cell is present within the subject. In an embodiment, the fluid comprises amniotic fluid, aqueous humor, vitreous humor, bile, blood, breast milk, cerebrospinal fluid, cerumen, chyle, cystic fluid, endolymph, feces, gastric acid, gastric juice, lymph, mucus, nipple aspirates, pericardial fluid, perilymph, peritoneal fluid, plasma, pleural fluid, pus, saliva, sebum, semen, sweat, serum, sputum, synovial fluid, tears, urine, vaginal secretions, or fluid collected from a biopsy. In one embodiment, the sample contains protein (e.g., proteins or peptides) from the subject. In another embodiment, the sample contains RNA (e.g., mRNA) from the subject or DNA (e.g., genomic DNA molecules) from the subject.

“Primary treatment” as used herein, refers to the initial treatment of a subject afflicted with a pervasive developmental disorder.

A pervasive developmental disorder is “treated” if at least one symptom of the pervasive developmental disorder is expected to be or is alleviated, terminated, slowed, or prevented. As used herein, a pervasive developmental disorder is also “treated” if recurrence or severity of the pervasive developmental disorder is reduced, slowed, delayed, or prevented.

A kit is any manufacture (e.g. a package or container) comprising at least one reagent, e.g. a probe, for specifically detecting a marker of the invention, the manufacture being promoted, distributed, or sold as a unit for performing the methods of the present invention.

“Metabolic pathway” refers to a sequence of enzyme-mediated reactions that transform one compound to another and provide intermediates and energy for cellular functions. The metabolic pathway can be linear or cyclic.

“Metabolic state” refers to the molecular content of a particular cellular, multicellular or tissue environment at a given point in time as measured by various chemical and biological indicators as they relate to a state of health or disease.

The term “microarray” refers to an array of distinct polynucleotides, oligonucleotides, polypeptides (e.g., antibodies) or peptides synthesized on a substrate, such as paper, nylon or other type of membrane, filter, chip, glass slide, or any other suitable solid support.

Antibodies used in immunoassays to determine the level of expression of one or more markers of the invention, may be labeled with a detectable label. The term “labeled”, with regard to the probe or antibody, is intended to encompass direct labeling of the probe or antibody by incorporation of a label (e.g., a radioactive atom), coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In one embodiment, the antibody is labeled, e.g. a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin), or an antibody fragment (e.g. a single-chain antibody, or an isolated antibody hypervariable domain) which binds specifically with the biomarker is used.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

The term “modulation” refers to upregulation (i.e., activation or stimulation), downregulation (i.e., inhibition or suppression) of a response, or the two in combination or apart. A “modulator” is a compound or molecule that modulates, and may be, e.g., an agonist, antagonist, activator, stimulator, suppressor, or inhibitor.

The term “genome” refers to the entirety of a biological entity's (cell, tissue, organ, system, organism) genetic information. It is encoded either in DNA or RNA (in certain viruses, for example). The genome includes both the genes and the non-coding sequences of the DNA.

The term “proteome” refers to the entire set of proteins expressed by a genome, a cell, a tissue, or an organism at a given time. More specifically, it may refer to the entire set of expressed proteins in a given type of cells or an organism at a given time under defined conditions. Proteome may include protein variants due to, for example, alternative splicing of genes and/or post-translational modifications (such as glycosylation or phosphorylation).

The term “transcriptome” refers to the entire set of transcribed RNA molecules, including mRNA, rRNA, tRNA, and other non-coding RNA produced in one or a population of cells at a given time. The term can be applied to the total set of transcripts in a given organism, or to the specific subset of transcripts present in a particular cell type. Unlike the genome, which is roughly fixed for a given cell line (excluding mutations), the transcriptome can vary with external environmental conditions. Because it includes all mRNA transcripts in the cell, the transcriptome reflects the genes that are being actively expressed at any given time, with the exception of mRNA degradation phenomena such as transcriptional attenuation.

The study of transcriptomics, also referred to as expression profiling, examines the expression level of mRNAs in a given cell population, often using high-throughput techniques based on DNA microarray technology.

The term “metabolome” refers to the complete set of small-molecule metabolites (such as metabolic intermediates, hormones and other signalling molecules, and secondary metabolites) to be found within a biological sample, such as a single organism, at a given time under a given condition. The metabolome is dynamic, and may change from second to second.

The term “interactome” refers to the whole set of molecular interactions in a biological system under study (e.g., cells). It can be displayed as a directed graph. Molecular interactions can occur between molecules belonging to different biochemical families (proteins, nucleic acids, lipids, carbohydrates, etc.) and also within a given family. When spoken in terms of proteomics, interactome refers to protein-protein interaction network (PPI), or protein interaction network (PIN). Another extensively studied type of interactome is the protein-DNA interactome (network formed by transcription factors (and DNA or chromatin regulatory proteins) and their target genes.

The term “cellular output” includes a collection of parameters, preferably measurable parameters, relating to cellular status, including (without limiting): level of transcription for one or more genes (e.g., measurable by RT-PCR, qPCR, microarray, etc.), level of expression for one or more proteins (e.g., measurable by mass spectrometry or Western blot), absolute activity (e.g., measurable as substrate conversion rates) or relative activity (e.g., measurable as a % value compared to maximum activity) of one or more enzymes or proteins, level of one or more metabolites or intermediates, level of oxidative phosphorylation (e.g., measurable by Oxigen Consumption Rate or OCR), level of glycolysis (e.g., measurable by Extra Cellular Acidification Rate or ECAR), extent of ligand-target binding or interaction, activity of extracellular secreted molecules, etc. The cellular output may include data for a pre-determined number of target genes or proteins, etc., or may include a global assessment for all detectable genes or proteins. For example, mass spectrometry may be used to identify and/or quantitate all detectable proteins expressed in a given sample or cell population, without prior knowledge as to whether any specific protein may be expressed in the sample or cell population.

As used herein, a “cell system” includes a population of homogeneous or heterogeneous cells. The cells within the system may be growing in vivo, under the natural or physiological environment, or may be growing in vitro in, for example, controlled tissue culture environments. The cells within the system may be relatively homogeneous (e.g., no less than 70%, 80%, 90%, 95%, 99%, 99.5%, 99.9% homogeneous), or may contain two or more cell types, such as cell types usually found to grow in close proximity in vivo, or cell types that may interact with one another in vivo through, e.g., paracrine or other long distance inter-cellular communication. The cells within the cell system may be derived from established cell lines, including pervasive developmental disorder cell lines, immortal cell lines, or normal cell lines, or may be primary cells or cells freshly isolated from live tissues or organs.

Cells in the cell system are typically in contact with a “cellular environment” that may provide nutrients, gases (oxygen or CO₂, etc.), chemicals, or proteinaceous/non-proteinaceous stimulants that may define the conditions that affect cellular behavior. The cellular environment may be a chemical media with defined chemical components and/or less well-defined tissue extracts or serum components, and may include a specific pH, CO₂ content, pressure, and temperature under which the cells grow. Alternatively, the cellular environment may be the natural or physiological environment found in vivo for the specific cell system.

In certain embodiments, a cellular environment for a specific cell system also include certain cell surface features of the cell system, such as the types of receptors or ligands on the cell surface and their respective activities, the structure of carbohydrate or lipid molecules, membrane polarity or fluidity, status of clustering of certain membrane proteins, etc. These cell surface features may affect the function of nearby cells, such as cells belonging to a different cell system. In certain other embodiments, however, the cellular environment of a cell system does not include cell surface features of the cell system.

The cellular environment may be altered to become a “modified cellular environment.” Alterations may include changes (e.g., increase or decrease) in any one or more component found in the cellular environment, including addition of one or more “external stimulus component” to the cellular environment. The external stimulus component may be endogenous to the cellular environment (e.g., the cellular environment contains some levels of the stimulant, and more of the same is added to increase its level), or may be exogenous to the cellular environment (e.g., the stimulant is largely absent from the cellular environment prior to the alteration). The cellular environment may further be altered by secondary changes resulting from adding the external stimulus component, since the external stimulus component may change the cellular output of the cell system, including molecules secreted into the cellular environment by the cell system.

As used herein, “external stimulus component” include any external physical and/or chemical stimulus that may affect cellular function. This may include any large or small organic or inorganic molecules, natural or synthetic chemicals, temperature shift, pH change, radiation, light (UVA, UVB etc.), microwave, sonic wave, electrical current, modulated or unmodulated magnetic fields, etc.

Merely to illustrate, the subject external stimulus component may include a therapeutic agent or a candidate therapeutic agent for treating a disease condition, including chemotherapeutic agent, protein-based biological drugs, antibodies, fusion proteins, small molecule drugs, lipids, polysaccharides, nucleic acids, etc.

In other embodiments, the external stimulus component may be one or more stress factors, such as those typically encountered in vivo under the various disease conditions, including hypoxia, hyperglycemic conditions, acidic environment (that may be mimicked by lactic acid treatment), etc.

In certain situations, where interaction between two or more cell systems are desired to be investigated, a “cross-talking cell system” may be formed by, for example, bringing the modified cellular environment of a first cell system into contact with a second cell system to affect the cellular output of the second cell system.

As used herein, “cross-talk cell system” comprises two or more cell systems, in which the cellular environment of at least one cell system comes into contact with a second cell system, such that at least one cellular output in the second cell system is changed or affected. In certain embodiments, the cell systems within the cross-talk cell system may be in direct contact with one another. In other embodiments, none of the cell systems are in direct contact with one another.

For example, in certain embodiments, the cross-talk cell system may be in the form of a transwell, in which a first cell system is growing in an insert and a second cell system is growing in a corresponding well compartment. The two cell systems may be in contact with the same or different media, and may exchange some or all of the media components. External stimulus component added to one cell system may be substantially absorbed by one cell system and/or degraded before it has a chance to diffuse to the other cell system. Alternatively, the external stimulus component may eventually approach or reach an equilibrium within the two cell systems.

In certain embodiments, the cross-talk cell system may adopt the form of separately cultured cell systems, where each cell system may have its own medium and/or culture conditions (temperature, CO₂ content, pH, etc.), or similar or identical culture conditions. The two cell systems may come into contact by, for example, taking the conditioned medium from one cell system and bringing it into contact with another cell system. Direct cell-cell contacts between the two cell systems can also be effected if desired. For example, the cells of the two cell systems may be co-cultured at any point if desired, and the co-cultured cell systems can later be separated by, for example, FACS sorting when cells in at least one cell system have a sortable marker or label (such as a stably expressed fluorescent marker protein GFP).

Similarly, in certain embodiments, the cross-talk cell system may simply be a co-culture. Selective treatment of cells in one cell system can be effected by first treating the cells in that cell system, before culturing the treated cells in co-culture with cells in another cell system. The co-culture cross-talk cell system setting may be helpful when it is desired to study, for example, effects on a second cell system caused by cell surface changes in a first cell system, after stimulation of the first cell system by an external stimulus component.

The cross-talk cell system of the invention is particularly suitable for exploring the effect of certain pre-determined external stimulus component on the cellular output of one or both cell systems. The primary effect of such a stimulus on the first cell system (with which the stimulus directly contact) may be determined by comparing cellular outputs (e.g., protein expression level) before and after the first cell system's contact with the external stimulus, which, as used herein, may be referred to as “(significant) cellular output differentials.” The secondary effect of such a stimulus on the second cell system, which is mediated through the modified cellular environment of the first cell system (such as it secretome), can also be similarly measured. There, a comparison in, for example, proteome of the second cell system can be made between the proteome of the second cell system with the external stimulus treatment on the first cell system, and the proteome of the second cell system without the external stimulus treatment on the first cell system. Any significant changes observed (in proteome or any other cellular outputs of interest) may be referred to as a “significant cellular cross-talk differential.”

In making cellular output measurements (such as protein expression), either absolute expression amount of relative expression level may be used. For example, to determine the relative protein expression level of a second cell system, the amount of any given protein in the second cell system, with or without the external stimulus to the first cell system, may be compared to a suitable control cell line and mixture of cell lines and given a fold-increase or fold-decrease value. A pre-determined threshold level for such fold-increase (e.g., at least 1.5 fold increase) or fold-decrease (e.g., at least a decrease to 0.75 fold or 75%) may be used to select significant cellular cross-talk differentials.

To illustrate, in one exemplary two-cell system established to imitate aspects of a cardiovascular disease model, a heart smooth muscle cell line (first cell system) may be treated with a hypoxia condition (an external stimulus component), and proteome changes in a kidney cell line (second cell system) resulting from contacting the kidney cells with conditioned medium of the heart smooth muscle may be measured using conventional quantitative mass spectrometry. Significant cellular cross-talking differentials in these kidney cells may be determined, based on comparison with a proper control (e.g., similarly cultured kidney cells contacted with conditioned medium from similarly cultured heart smooth muscle cells not treated with hypoxia conditions).

Not every observed significant cellular cross-talking differentials may be of biological significance. With respect to any given biological system for which the subject interrogative biological assessment is applied, some (or maybe all) of the significant cellular cross-talking differentials may be “determinative” with respect to the specific biological problem at issue, e.g., either responsible for causing a disease condition (a potential target for therapeutic intervention) or is a biomarker for the disease condition (a potential diagnostic or prognostic factor).

Such determinative cross-talking differentials may be selected by an end user of the subject method, or it may be selected by a bioinformatics software program, such as DAVID-enabled comparative pathway analysis program, or the KEGG pathway analysis program. In certain embodiments, more than one bioinformatics software program is used, and consensus results from two or more bioinformatics software programs are preferred.

As used herein, “differentials” of cellular outputs include differences (e.g., increased or decreased levels) in any one or more parameters of the cellular outputs. For example, in terms of protein expression level, differentials between two cellular outputs, such as the outputs associated with a cell system before and after the treatment by an external stimulus component, can be measured and quantitated by using art-recognized technologies, such as mass-spectrometry based assays (e.g., iTRAQ, 2D-LC-MSMS, etc.).

As used herein, an “interrogative biological assessment” may include the identification of one or more determinative cellular cross-talk differentials (e.g., an increase or decrease in activity of a biological pathway, or key members of the pathway, or key regulators to members of the pathway) associated with the external stimulus component. It may further include additional steps designed to test or verify whether the identified determinative cellular cross-talk differentials are necessary and/or sufficient for the downstream events associated with the initial external stimulus component, including in vivo animal models and/or in vitro tissue culture experiments.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### II. Overview of Interrogative Biology Platform Technology

Exemplary embodiments of the present invention incorporate methods that may be performed using an interrogative biology platform (“the Platform”) that is a tool for understanding a wide variety of biological processes, such as disease pathophysiology, and the key molecular drivers underlying such biological processes, including factors that enable a disease process. Some exemplary embodiments include systems that may incorporate at least a portion of, or all of, the Platform. Some exemplary methods may employ at least some of, or all of the Platform. Goals and objectives of some exemplary embodiments involving the platform are generally outlined below for illustrative purposes:

i) to create specific molecular signatures as drivers of critical components of the disease process (e.g., pervasive developmental disorder) as they relate to overall pathophysiology of the disease process;

ii) to generate molecular signatures or differential maps pertaining to the disease process, e.g., pervasive developmental disorder, which may help to identify differential molecular signatures that distinguishes the disease state versus a different state (e.g., a normal state), and develop understanding of signatures or molecular entities as they arbitrate mechanisms of change between the two states (e.g., from normal to disease state); and,

iii) to investigate the role of “hubs” of molecular activity as potential intervention targets for external control of the disease, e.g., pervasive developmental disorder, (e.g., to use the hub as a potential therapeutic target), or as potential bio-markers for the disease, e.g., pervasive developmental disorder, in question (e.g., disease specific biomarkers, in prognostic and/or theranostics uses).

Some exemplary methods involving the Platform may include one or more of the following features:

1) modeling the biological process (e.g., disease process) and/or components of the biological process (e.g., disease physiology & pathophysiology) in one or more models, preferably in vitro models, using cells associated with the biological process. For example, the cells may be human derived cells which normally participate in the biological process in question. The model may include various cellular cues/conditions/perturbations that are specific to the biological process (e.g., disease). Ideally, the model represents various (disease) states and flux components, instead of a static assessment of the biological (disease) condition.

2) profiling mRNA and/or protein signatures using any art-recognized means. For example, quantitative polymerase chain reaction (qPCR) & proteomics analysis tools such as Mass Spectrometry (MS). Such mRNA and protein data sets represent biological reaction to environment/perturbation. Where applicable and possible, lipidomics, metabolomics, and transcriptomics data may also be integrated as supplemental or alternative measures for the biological process in question. SNP analysis is another component that may be used at times in the process. It may be helpful for investigating, for example, whether the SNP or a specific mutation has any effect on the biological process. These variables may be used to describe the biological process, either as a static “snapshot,” or as a representation of a dynamic process.

3) assaying for one or more cellular responses to cues and perturbations, including but not limited to bioenergetics profiling, cell proliferation, apoptosis, and organellar function. True genotype-phenotype association is actualized by employment of functional models, such as ATP, ROS, OXPHOS, Seahorse assays, etc. Such cellular responses represent the reaction of the cells in the biological process (or models thereof) in response to the corresponding state(s) of the mRNA/protein expression, and any other related states in 2) above.

4) integrating functional assay data thus obtained in 3) with proteomics and other data obtained in 2), and determining protein associations as driven by causality, by employing artificial intelligence based (AI-based) informatics system or platform. Such an AI-based system is based on, and preferably based only on, the data sets obtained in 2) and/or 3), without resorting to existing knowledge concerning the biological process. Preferably, no data points are statistically or artificially cut-off. Instead, all obtained data is fed into the AI-system for determining protein associations. One goal or output of the integration process is one or more differential networks (otherwise may be referred to herein as “delta networks,” or, in some cases, “delta-delta networks” as the case may be) between the different biological states (e.g., disease vs. normal states).

5) profiling the outputs from the AI-based informatics platform to explore each hub of activity as a potential therapeutic target and/or biomarker. Such profiling can be done entirely in silico based on the obtained data sets, without resorting to any actual wet-lab experiments.

6) validating hub of activity by employing molecular and cellular techniques. Such post-informatic validation of output with wet-lab cell-based experiments may be optional, but they help to create a full-circle of interrogation.

Any or all of the approaches outlined above may be used in any specific application concerning any biological process, depending, at least in part, on the nature of the specific application. That is, one or more approaches outlined above may be omitted or modified, and one or more additional approaches may be employed, depending on specific application.

A schematic representation of the components of the platform including data collection, data integration, and data mining is depicted in FIG. 2. A schematic representation of a systematic interrogation and collection of response data from the “omics” cascade is depicted in FIG. 1.

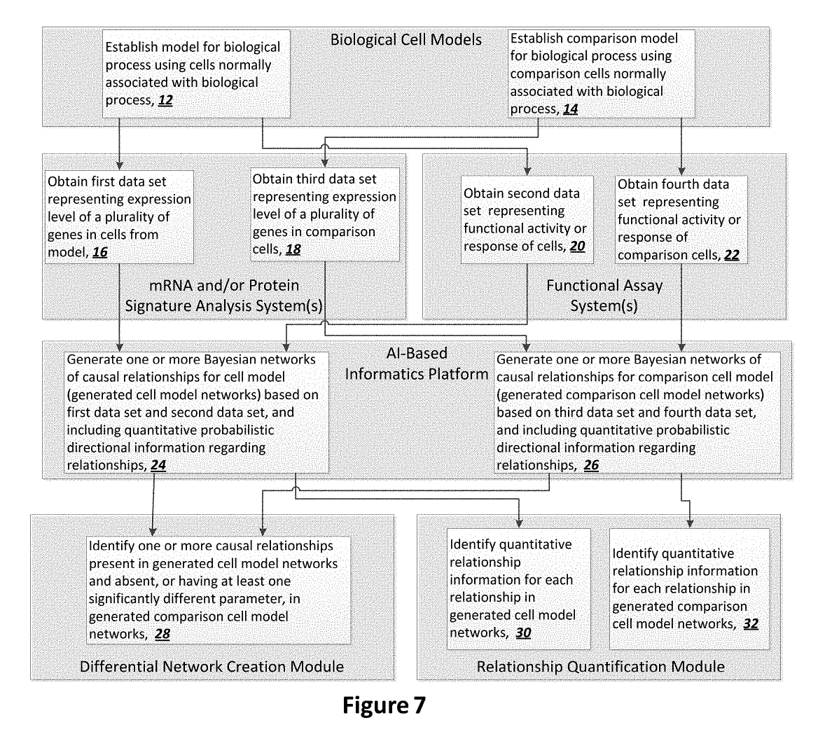

FIG. 7 is a high level flow chart of an exemplary method, in which components of an exemplary system that may be used to perform the exemplary method are indicated. Initially, a model (e.g., an in vitro model) is established for a biological process (e.g., a disease process) and/or components of the biological process (e.g., disease physiology and pathophysiology) using cells normally associated with the biological process (step 12). For example, the cells may be human-derived cells that normally participate in the biological process (e.g., disease). The cell model may include various cellular cues, conditions, and/or perturbations that are specific to the biological process (e.g., disease). Ideally, the cell model represents various (disease) states and flux components of the biological process (e.g., disease), instead of a static assessment of the biological process. The comparison cell model may include control cells or normal (e.g., non-diseased) cells. Additional description of the cell models appears below in sections IV.A.

A first data set is obtained from the cell model for the biological process, which includes information representing expression levels of a plurality of genes (e.g., mRNA and/or protein signatures) (step 16) using any known process or system (e.g., quantitative polymerase chain reaction (qPCR) & proteomics analysis tools such as Mass Spectrometry (MS)).

A third data set is obtained from the comparison cell model for the biological process (step 18). The third data set includes information representing expression levels of a plurality of genes in the comparison cells from the comparison cell model.

In certain embodiments of the methods of the invention, these first and third data sets are collectively referred to herein as a “first data set” that represents expression levels of a plurality of genes in the cells (all cells including comparison cells) associated with the biological system.

The first data set and third data set may be obtained from one or more mRNA and/or Protein Signature Analysis System(s). The mRNA and protein data in the first and third data sets may represent biological reactions to environment and/or perturbation. Where applicable and possible, lipidomics, metabolomics, and transcriptomics data may also be integrated as supplemental or alternative measures for the biological process. The SNP analysis is another component that may be used at times in the process. It may be helpful for investigating, for example, whether a single-nucleotide polymorphism (SNP) or a specific mutation has any effect on the biological process. The data variables may be used to describe the biological process, either as a static “snapshot,” or as a representation of a dynamic process. Additional description regarding obtaining information representing expression levels of a plurality of genes in cells appears below in section IV.B.

In certain embodiments, a second data set is obtained from the cell model for the biological process, which includes information representing a functional activity or response of cells (step 20). Similarly, in certain embodiments, a fourth data set is obtained from the comparison cell model for the biological process, which includes information representing a functional activity or response of the comparison cells (step 22).

In certain embodiments of the methods of the invention, these second and fourth data sets are collectively referred to herein as a “second data set” that represents a functional activity or a cellular response of the cells (all cells including comparison cells) associated with the biological system.

One or more functional assay systems may be used to obtain information regarding the functional activity or response of cells or of comparison cells. The information regarding functional cellular responses to cues and perturbations may include, but is not limited to, bioenergetics profiling, cell proliferation, apoptosis, and organellar function. Functional models for processes and pathways (e.g., adenosine triphosphate (ATP), reactive oxygen species (ROS), oxidative phosphorylation (OXPHOS), Seahorse assays, etc.,) may be employed to obtain true genotype-phenotype association. The functional activity or cellular responses represent the reaction of the cells in the biological process (or models thereof) in response to the corresponding state(s) of the mRNA/protein expression, and any other related applied conditions or perturbations. Additional information regarding obtaining information representing functional activity or response of cells is provided below in section IV.B.

The method also includes generating computer-implemented models of the biological processes in the cells and in the control cells. For example, one or more (e.g., an ensemble of) Bayesian networks of causal relationships between the expression level of the plurality of genes and the functional activity or cellular response may be generated for the cell model (the “generated cell model networks”) from the first data set and the second data set (step 24). The generated cell model networks, individually or collectively, include quantitative probabilistic directional information regarding relationships. The generated cell model networks are not based on known biological relationships between gene expression and/or functional activity or cellular response, other than information from the first data set and second data set. The one or more generated cell model networks may collectively be referred to as a consensus cell model network.

One or more (e.g., an ensemble of) Bayesian networks of causal relationships between the expression level of the plurality of genes and the functional activity or cellular response may be generated for the comparison cell model (the “generated comparison cell model networks”) from the first data set and the second data set (step 26). The generated comparison cell model networks, individually or collectively, include quantitative probabilistic directional information regarding relationships. The generated cell networks are not based on known biological relationships between gene expression and/or functional activity or cellular response, other than the information in the first data set and the second data set. The one or more generated comparison model networks may collectively be referred to as a consensus cell model network.

The generated cell model networks and the generated comparison cell model networks may be created using an artificial intelligence based (AI-based) informatics platform. Further details regarding the creation of the generated cell model networks, the creation of the generated comparison cell model networks and the AI-based informatics system appear below in section IV.C.

It should be noted that many different AI-based platforms or systems may be employed to generate the Bayesian networks of causal relationships including quantitative probabilistic directional information. Although certain examples described herein employ one specific commercially available system, i.e., REFS™ (Reverse Engineering/Forward Simulation) from GNS (Cambridge, Mass.), embodiments are not limited. AI-Based Systems or Platforms suitable to implement some embodiments employ mathematical algorithms to establish causal relationships among the input variables (e.g., the first and second data sets), based only on the input data without taking into consideration prior existing knowledge about any potential, established, and/or verified biological relationships.

For example, the REFS™ AI-based informatics platform utilizes experimentally derived raw (original) or minimally processed input biological data (e.g., genetic, genomic, epigenetic, proteomic, metabolomic, and clinical data), and rapidly performs trillions of calculations to determine how molecules interact with one another in a complete system. The REFS™ AI-based informatics platform performs a reverse engineering process aimed at creating an in silico computer-implemented cell model (e.g., generated cell model networks), based on the input data, that quantitatively represents the underlying biological system. Further, hypotheses about the underlying biological system can be developed and rapidly simulated based on the computer-implemented cell model, in order to obtain predictions, accompanied by associated confidence levels, regarding the hypotheses.

With this approach, biological systems are represented by quantitative computer-implemented cell models in which “interventions” are simulated to learn detailed mechanisms of the biological system (e.g., disease), effective intervention strategies, and/or clinical biomarkers that determine which patients will respond to a given treatment regimen. Conventional bioinformatics and statistical approaches, as well as approaches based on the modeling of known biology, are typically unable to provide these types of insights.

After the generated cell model networks and the generated comparison cell model networks are created, they are compared. One or more causal relationships present in at least some of the generated cell model networks, and absent from, or having at least one significantly different parameter in, the generated comparison cell model networks are identified (step 28). Such a comparison may result in the creation of a differential network. The comparison, identification, and/or differential (delta) network creation may be conducted using a differential network creation module, which is described in further detail below in section IV.D.

In some embodiments, input data sets are from one cell type and one comparison cell type, which creates an ensemble of cell model networks based on the one cell type and another ensemble of comparison cell model networks based on the one comparison control cell type. A differential may be performed between the ensemble of networks of the one cell type and the ensemble of networks of the comparison cell type(s).

In other embodiments, input data sets are from multiple cell types and multiple comparison cell types. An ensemble of cell model networks may be generated for each cell types and each comparison cell type individually, and/or data from the multiple cell types and the multiple comparison cell types may be combined into respective composite data sets. The composite data sets produce an ensemble of networks corresponding to the multiple cell types (composite data) and another ensemble of networks corresponding to the multiple comparison cell types (comparison composite data). A differential may be performed on the ensemble of networks for the composite data as compared to the ensemble of networks for the comparison composite data.

In some embodiments, a differential may be performed between two different differential networks. This output may be referred to as a delta-delta network.

Quantitative relationship information may be identified for each relationship in the generated cell model networks (step 30). Similarly, quantitative relationship information for each relationship in the generated comparison cell model networks may be identified (step 32). The quantitative information regarding the relationship may include a direction indicating causality, a measure of the statistical uncertainty regarding the relationship (e.g., an Area Under the Curve (AUC) statistical measurement), and/or an expression of the quantitative magnitude of the strength of the relationship (e.g., a fold). The various relationships in the generated cell model networks may be profiled using the quantitative relationship information to explore each hub of activity in the networks as a potential therapeutic target and/or biomarker. Such profiling can be done entirely in silico based on the results from the generated cell model networks, without resorting to any actual wet-lab experiments.

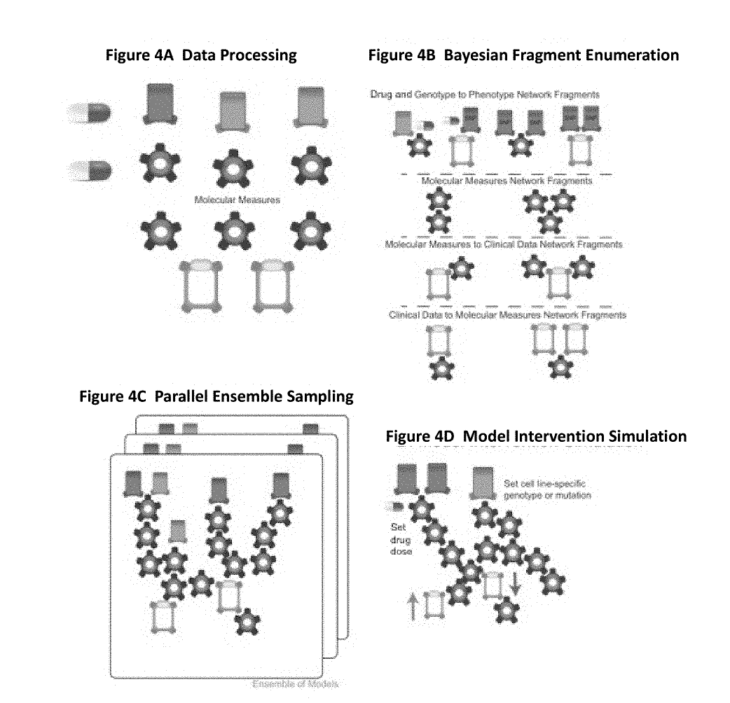

In some embodiments, a hub of activity in the networks may be validated by employing molecular and cellular techniques. Such post-informatic validation of output with wet-lab cell based experiments need not be performed, but it may help to create a full-circle of interrogation. FIG. 4 schematically depicts a simplified high level representation of the functionality of an exemplary AI-based informatics system (e.g., REFS™ AI-based informatics system) and interactions between the AI-based system and other elements or portions of an interrogative biology platform (“the Platform”). In FIG. 4A, various data sets obtained from a model for a biological process (e.g., a disease model), such as drug dosage, treatment dosage, protein expression, mRNA expression, and any of many associated functional measures (such as OCR, ECAR) are fed into an AI-based system. As shown in FIG. 4B, from the input data sets, the AI-system creates a library of “network fragments” that includes variables (proteins, lipids and metabolites) that drive molecular mechanisms in the biological process (e.g., disease), in a process referred to as Bayesian Fragment Enumeration (FIG. 4B).

In FIG. 4C, the AI-based system selects a subset of the network fragments in the library and constructs an initial trial network from the fragments. The AI-based system also selects a different subset of the network fragments in the library to construct another initial trial network. Eventually an ensemble of initial trial networks are created (e.g., 1000 networks) from different subsets of network fragments in the library. This process may be termed parallel ensemble sampling. Each trial network in the ensemble is evolved or optimized by adding, subtracting and/or substitution additional network fragments from the library. If additional data is obtained, the additional data may be incorporated into the network fragments in the library and may be incorporated into the ensemble of trial networks through the evolution of each trial network. After completion of the optimization/evolution process, the ensemble of trial networks may be described as the generated cell model networks.

As shown in FIG. 4D, the ensemble of generated cell model networks may be used to simulate the behavior of the biological system. The simulation may be used to predict behavior of the biological system to changes in conditions, which may be experimentally verified using wet-lab cell-based, or animal-based, experiments. Also, quantitative parameters of relationships in the generated cell model networks may be extracted using the simulation functionality by applying simulated perturbations to each node individually while observing the effects on the other nodes in the generated cell model networks. Further detail is provided below in section IV.C.

The automated reverse engineering process of the AI-based informatics system creates an ensemble of generated cell model networks that is an unbiased and systematic computer-based model of the cells.

The reverse engineering determines the probabilistic directional network connections between the molecular measurements in the data, and the phenotypic outcomes of interest. The variation in the molecular measurements enables learning of the probabilistic cause and effect relationships between these entities and changes in endpoints. The machine learning nature of the platform also enables cross training and predictions based on a data set that is constantly evolving.

The network connections between the molecular measurements in the data are “probabilistic,” partly because the connection may be based on correlations between the observed data sets “learned” by the computer algorithm. For example, if the expression level of protein X and that of protein Y are positively or negatively correlated, based on statistical analysis of the data set, a causal relationship may be assigned to establish a network connection between proteins X and Y. The reliability of such a putative causal relationship may be further defined by a likelihood of the connection, which can be measured by p-value (e.g., p<0.1, 0.05, 0.01, etc).

The network connections between the molecular measurements in the data are “directional,” partly because the network connections between the molecular measurements, as determined by the reverse-engineering process, reflects the cause and effect of the relationship between the connected gene/protein, such that raising the expression level of one protein may cause the expression level of the other to rise or fall, depending on whether the connection is stimulatory or inhibitory.

The network connections between the molecular measurements in the data are “quantitative,” partly because the network connections between the molecular measurements, as determined by the process, may be simulated in silico, based on the existing data set and the probabilistic measures associated therewith. For example, in the established network connections between the molecular measurements, it may be possible to theoretically increase or decrease (e.g., by 1, 2, 3, 5, 10, 20, 30, 50,100-fold or more) the expression level of a given protein (or a “node” in the network), and quantitatively simulate its effects on other connected proteins in the network.

The network connections between the molecular measurements in the data are “unbiased,” at least partly because no data points are statistically or artificially cut-off, and partly because the network connections are based on input data alone, without referring to pre-existing knowledge about the biological process in question.

The network connections between the molecular measurements in the data are “systemic” and (unbiased), partly because all potential connections among all input variables have been systemically explored, for example, in a pair-wise fashion. The reliance on computing power to execute such systemic probing exponentially increases as the number of input variables increases.

In general, an ensemble of ˜1,000 networks is usually sufficient to predict probabilistic causal quantitative relationships among all of the measured entities. The ensemble of networks captures uncertainty in the data and enables the calculation of confidence metrics for each model prediction. Predictions generated using the ensemble of networks together, where differences in the predictions from individual networks in the ensemble represent the degree of uncertainty in the prediction. This feature enables the assignment of confidence metrics for predictions of clinical response generated from the model.

Once the models are reverse-engineered, further simulation queries may be conducted on the ensemble of models to determine key molecular drivers for the biological process in question, such as a disease condition.

### III. Exemplary Steps and Components of the Platform Technology

For illustration purpose only, the following steps of the subject Platform Technology may be described herein below for integrating data obtained from a custom built pervasive developmental disorder model, and for identifying novel proteins/pathways driving the pathogenesis of pervasive developmental disorder. Relational maps resulting from this analysis provides pervasive developmental disorder treatment targets, as well as diagnostic/prognostic markers associated with pervasive developmental disorder. Methods described here are described in further detail in U.S. Ser. No. 13/411,460, the entire contents of which are expressly incorporated herein by reference.

In addition, although the description below is presented in some portions as discrete steps, it is for illustration purpose and simplicity, and thus, in reality, it does not imply such a rigid order and/or demarcation of steps. Moreover, the steps of the invention may be performed separately, and the invention provided herein is intended to encompass each of the individual steps separately, as well as combinations of one or more (e.g., any one, two, three, four, five, six or all seven steps) steps of the subject Platform Technology, which may be carried out independently of the remaining steps.

The invention also is intended to include all aspects of the Platform Technology as separate components and embodiments of the invention. For example, the generated data sets are intended to be embodiments of the invention. As further examples, the generated causal relationship networks, generated consensus causal relationship networks, and/or generated simulated causal relationship networks, are also intended to be embodiments of the invention. The causal relationships identified as being unique in a pervasive developmental disorder are intended to be embodiments of the invention. Further, the custom built models for a pervasive developmental disorder are also intended to be embodiments of the invention.

A. Custom Model Building

The first step in the Platform Technology is the establishment of a model for a biological system or process, e.g., a pervasive developmental disorder. An example of a pervasive developmental disorder is autism. As any other complicated biological process or system, autism is a complicated pathological condition characterized by multiple unique aspects. For example, mitochondrial dysfunction may play a crucial role in the autism disease pathophysiology. As a result, autism cells may react differently to an environmental perturbation associated with mitochondrial functions, such as treatment by a potential drug, as compared to the reaction by a normal cell in response to the same treatment. Thus, it would be of interest to decipher autism's unique responses to drug treatment as compared to the responses of normal cells. To this end, a custom autism model may be established to simulate the environment of a cell associated with the autism disorder, e.g., lymphoblasts or other bodily fluid (e.g. serum or urine) samples from autism patients. Environmental perturbations associated with mitochondrial functions, e.g. CoQ10, can be applied to treat the autism cells. Mitochondrial function assays, e.g ATP and/or ROS, can be employed to provide insightful biological readout.

Individual conditions reflecting different aspects or characteristics of a pervasive developmental disorder may be investigated separately in the custom built pervasive developmental disorder model, and/or may be combined together. In one embodiment, combinations of at least 2, 3, 4, 5, 6, 7, 8, 9, 10, 15, 20, 25, 30, 40, 50 or more conditions reflecting or simulating different aspects of pervasive developmental disorder are investigated in the custom built pervasive developmental disorder model. In one embodiment, individual conditions and, in addition, combinations of at least 2, 3, 4, 5, 6, 7, 8, 9, 10, 15, 20, 25, 30, 40, 50 or more of the conditions reflecting or simulating different aspects of pervasive developmental disorder are investigated in the custom built pervasive developmental disorder model. All values presented in the foregoing list can also be the upper or lower limit of ranges, that are intended to be a part of this invention, e.g., between 1 and 5, 1 and 10, 1 and 20, 1 and 30, 2 and 5, 2 and 10, 5 and 10, 1 and 20, 5 and 20, 10 and 20, 10 and 25, 10 and 30 or 10 and 50 different conditions.

As a control one or more normal cell lines (e.g., cells obtained from normal, unaffected subjects, e.g., normal, unaffected subjects that are family members of a subject suffering from a pervasive developmental disorder and from which the cells associated with a pervasive developmental disorder are obtained) are cultured under similar conditions in order to identify proteins or pathways unique to a pervasive developmental disorder (see below).

Multiple cell types from the same subject afflicted with or suffering from a pervasive developmental disorder, e.g., lymphoblasts and cells derived from the central nervous system, or cells from multiple different subjects afflicted with or suffering from a pervasive developmental disorder, may be included in the pervasive developmental disorder model. In certain situations, cross talk or ECS experiments between different cells associated with a pervasive developmental disorder model may be conducted for several inter-related purposes.

In some embodiments that involve cross talk, experiments conducted on the cell models are designed to determine modulation of cellular state or function of one cell system or population (e.g., lymphoblasts) by another cell system or population (e.g., cells derived from the central nervous system), optionally under defined treatment conditions. According to a typical setting, a first cell system/population is contacted by an external stimulus components, such as a candidate molecule (e.g., a small drug molecule, a protein) or a candidate condition (e.g., hypoxia, high glucose environment). In response, the first cell system/population changes its transcriptome, proteome, metabolome, and/or interactome, leading to changes that can be readily detected both inside and outside the cell. For example, changes in transcriptome can be measured by the transcription level of a plurality of target mRNAs; changes in proteome can be measured by the expression level of a plurality of target proteins; and changes in metabolome can be measured by the level of a plurality of target metabolites by assays designed specifically for given metabolites. Alternatively, the above referenced changes in metabolome and/or proteome, at least with respect to certain secreted metabolites or proteins, can also be measured by their effects on the second cell system/population, including the modulation of the transcriptome, proteome, metabolome, and interactome of the second cell system/population. Therefore, the experiments can be used to identify the effects of the molecule(s) of interest secreted by the first cell system/population on a second cell system/population under different treatment conditions. The experiments can also be used to identify any proteins that are modulated as a result of signaling from the first cell system (in response to the external stimulus component treatment) to another cell system, by, for example, differential screening of proteomics. The same experimental setting can also be adapted for a reverse setting, such that reciprocal effects between the two cell systems can also be assessed. In general, for this type of experiment, the choice of cell line pairs is largely based on the factors such as origin, disease state and cellular function.

Although two-cell systems are typically involved in this type of experimental setting, similar experiments can also be designed for more than two cell systems by, for example, immobilizing each distinct cell system on a separate solid support.

Once the custom model is built, one or more “perturbations” may be applied to the system, such as genetic variation from patient to patient, or with/without treatment by certain drugs or pro-drugs. The effects of such perturbations to the system, including the effect on pervasive developmental disorder related cells, and normal control cells, can be measured using various art-recognized or proprietary means, as described in section IV.B below.

In an exemplary embodiment, cell lines derived from one or more subjects afflicted with a pervasive developmental disorder, e.g., autism, and control, e.g., normal cells, e.g., cells derived from unaffected subjects, such as one or more unaffected family members related to the subject afflicted with a pervasive developmental disorder, are used. In one embodiment, the cells are treated with or without an environmental perturbation, e.g., treatment with Coenzyme Q10.

The custom built pervasive developmental disorder model may be established and used throughout the steps of the Platform Technology of the invention to ultimately identify a causal relationship unique in the pervasive developmental disorder, by carrying out the steps described herein. It will be understood by the skilled artisan, however, that a custom built pervasive developmental disorder model that is used to generate an initial, “first generation” consensus causal relationship network for a pervasive developmental disorder can continually evolve or expand over time, e.g., by the introduction of additional cell lines and/or additional appropriate conditions. Additional data from the evolved cell model for a pervasive developmental disorder, i.e., data from the newly added portion(s) of the cell model, can be collected. The new data collected from an expanded or evolved cell model, i.e., from newly added portion(s) of the cell model, can then be introduced to the data sets previously used to generate the “first generation” consensus causal relationship network in order to generate a more robust “second generation” consensus causal relationship network. New causal relationships unique to the pervasive developmental disorder can then be identified from the “second generation” consensus causal relationship network. In this way, the evolution of the cell model provides an evolution of the consensus causal relationship networks, thereby providing new and/or more reliable insights into the modulators of the pervasive developmental disorder.

The present invention provides methods that include treating cells with an Environmental Influencer. “Environmental influencers” (Env-influencers) are molecules that influence or modulate the disease environment of a human in a beneficial manner allowing the human's disease environment to shift, reestablish back to or maintain a normal or healthy environment leading to a normal state. Env-influencers include both Multidimensional Intracellular Molecules (MIMs) and Epimetabolic shifters (Epi-shifters) as defined below. MIMs and epishifters are described in further detail in U.S. Ser. No. 12/777,902 (US 2011-0110914), the entire contents of which are expressly incorporated herein by reference.

The term “Multidimensional Intracellular Molecule (MIM)” is an isolated version or synthetically produced version of an endogenous molecule that is naturally produced by the body and/or is present in at least one cell of a human. A MIM is characterized by one or more, two or more, three or more, or all of the following functions. MIMs are capable of entering a cell, and the entry into the cell includes complete or partial entry into the cell, as long as the biologically active portion of the molecule wholly enters the cell. MIMs are capable of inducing a signal transduction and/or gene expression mechanism within a cell. MIMs are multidimensional in that the molecules have both a therapeutic and a carrier, e.g., drug delivery, effect. MIMs also are multidimensional in that the molecules act one way in a disease state and a different way in a normal state. Preferably, MIMs selectively act in cells of a disease state, and have substantially no effect in (matching) cells of a normal state. Preferably, MIMs selectively renders cells of a disease state closer in phenotype, metabolic state, genotype, mRNA/protein expression level, etc. to (matching) cells of a normal state.

In one embodiment, a MIM is also an epi-shifter. In another embodiment, a MIM is not an epi-shifter. The skilled artisan will appreciate that a MIM of the invention is also intended to encompass a mixture of two or more endogenous molecules, wherein the mixture is characterized by one or more of the foregoing functions. The endogenous molecules in the mixture are present at a ratio such that the mixture functions as a MIM.

MIMs can be lipid based or non-lipid based molecules. Examples of MIMs include, but are not limited to, CoQ10, acetyl Co-A, palmityl Co-A, L-carnitine, amino acids such as, for example, tyrosine, phenylalanine, and cysteine. In one embodiment, the MIM is a small molecule. In one embodiment of the invention, the MIM is not CoQ10. MIMs can be routinely identified by one of skill in the art using any of the assays described in detail herein.

As used herein, an “epimetabolic shifter” (epi-shifter) is a molecule (endogenous or exogenous) that modulates the metabolic shift from a healthy (or normal) state to a disease state and vice versa, thereby maintaining or reestablishing cellular, tissue, organ, system and/or host health in a human. Epi-shifters are capable of effectuating normalization in a tissue microenvironment. For example, an epi-shifter includes any molecule which is capable, when added to or depleted from a cell, of affecting the microenvironment (e.g., the metabolic state) of a cell. The skilled artisan will appreciate that an epi-shifter of the invention is also intended to encompass a mixture of two or more molecules, wherein the mixture is characterized by one or more of the foregoing functions. The molecules in the mixture are present at a ratio such that the mixture functions as an epi-shifter.

In some embodiments, the epi-shifter is an enzyme, such as an enzyme that either directly participates in catalyzing one or more reactions in the Citric Acid Cycle, or produces a Citric Acid Cycle intermediate, the excess of which drive the Citric Acid Cycle. In one embodiment, the enzyme is a component enzyme or enzyme complex that facilitates the Citric Acid Cycle, such as a synthase or a ligase. Exemplary enzymes include succinyl CoA synthase (Krebs Cycle enzyme) or pyruvate carboxylase (a ligase that catalyzes the reversible carboxylation of pyruvate to form oxaloacetate (OAA), a Krebs Cycle intermediate).

In some embodiments, the enzymes of the present invention, e.g., the MIMs or epi-shifters described herein, share a common activity with the proteins listed in Tables 2-6. As used herein, the phrase “share a common activity with a protein listed in Tables 2-6” refers to the ability of a protein to exhibit at least a portion of the same or similar activity as said protein. In some embodiments, the proteins of the present invention exhibit 25% or more of the activity of said protein. In some embodiments, the compounds of the present invention exhibit up to and including about 130% of the activity of said protein. In some embodiments, the compounds of the present invention exhibit about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, or 130% of the activity of said protein. It is to be understood that each of the values listed in this paragraph may be modified by the term “about.” Additionally, it is to be understood that any range which is defined by any two values listed in this paragraph is meant to be encompassed by the present invention. For example, in some embodiments, the proteins of the present invention exhibit between about 50% and about 100% of the activity of said protein.

B. Data Collection

In general, two types of data may be collected from any custom built model system for a pervasive developmental disorder. One type of data (e.g., the first set of data, the third set of data) usually relates to the level of certain macromolecules, such as DNA, RNA, protein, lipid, etc. An exemplary data set in this category is proteomic data (e.g., qualitative and quantitative data concerning the expression of all or substantially all measurable proteins from a sample). Another type of data that may, optionally, be collected is functional data (e.g., the optional second set of data, the fourth set of data) that reflects the phenotypic changes resulting from the changes in the first type of data.

With respect to the first type of data, in some example embodiments, quantitative polymerase chain reaction (qPCR) and proteomics are performed to profile changes in cellular mRNA and protein expression by quantitative polymerase chain reaction (qPCR) and proteomics. Total RNA can be isolated using a commercial RNA isolation kit. Following cDNA synthesis, specific commercially available qPCR arrays (e.g., those from SA Biosciences) for disease area or cellular processes such as angiogenesis, apoptosis, and diabetes, may be employed to profile a predetermined set of genes by following a manufacturer's instructions. For example, the Biorad cfx-384 amplification system can be used for all transcriptional profiling experiments. Following data collection (Ct), the final fold change over control can be determined using the δCt method as outlined in manufacturer's protocol. Proteomic sample analysis can be performed as described in subsequent sections.

The subject method may employ large-scale high-throughput quantitative proteomic analysis of hundreds of samples of similar character, and provides the data necessary for identifying the cellular output differentials.

There are numerous art-recognized technologies suitable for this purpose. An exemplary technique, iTRAQ analysis in combination with mass spectrometry, is briefly described below.

The quantitative proteomics approach is based on stable isotope labeling with the 8-plex iTRAQ reagent and 2D-LC MALDI MS/MS for peptide identification and quantification. Quantification with this technique is relative: peptides and proteins are assigned abundance ratios relative to a reference sample. Common reference samples in multiple iTRAQ experiments facilitate the comparison of samples across multiple iTRAQ experiments.

For example, to implement this analysis scheme, six primary samples and two control pool samples can be combined into one 8-plex iTRAQ mix according to the manufacturer's suggestions. This mixture of eight samples then can be fractionated by two-dimensional liquid chromatography; strong cation exchange (SCX) in the first dimension, and reversed-phase HPLC in the second dimension, then can be subjected to mass spectrometric analysis.

A brief overview of exemplary laboratory procedures that can be employed is provided herein.

Protein Extraction:

Cells can be lysed with 8 M urea lysis buffer with protease inhibitors (Thermo Scientific Halt Protease inhibitor EDTA-free) and incubate on ice for 30 minutes with vertex for 5 seconds every 10 minutes. Lysis can be completed by ultrasonication in 5 seconds pulse. Cell lysates can be centrifuged at 14000×g for 15 minutes (4° C.) to remove cellular debris. Bradford assay can be performed to determine the protein concentration. 100 ug protein from each samples can be reduced (10 mM Dithiothreitol (DTT), 55° C., 1 h), alkylated (25 mM iodoacetamide, room temperature, 30 minutes) and digested with Trypsin (1:25 w/w, 200 mM triethylammonium bicarbonate (TEAB), 37° C., 16 h).

Secretome Sample Preparation:

1) In one embodiment, the cells can be cultured in serum free medium: Conditioned media can be concentrated by freeze dryer, reduced (10 mM Dithiothreitol (DTT), 55° C., 1 h), alkylated (25 mM iodoacetamide, at room temperature, incubate for 30 minutes), and then desalted by actone precipitation. Equal amount of proteins from the concentrated conditioned media can be digested with Trypsin (1:25 w/w, 200 mM triethylammonium bicarbonate (TEAB), 37° C., 16 h).

In one embodiment, the cells can be cultured in serum containing medium: The volume of the medium can be reduced using 3 k MWCO Vivaspin columns (GE Healthcare Life Sciences), then can be reconstituted with 1×PBS (Invitrogen). Serum albumin can be depleted from all samples using AlbuVoid column (Biotech Support Group, LLC) following the manufacturer's instructions with the modifications of buffer-exchange to optimize for condition medium application.

iTRAQ 8 Plex Labeling:

Aliquot from each tryptic digests in each experimental set can be pooled together to create the pooled control sample. Equal aliquots from each sample and the pooled control sample can be labeled by iTRAQ 8 Plex reagents according to the manufacturer's protocols (AB Sciex). The reactions can be combined, vacuumed to dryness, re-suspended by adding 0.1% formic acid, and analyzed by LC-MS/MS.

2D-NanoLC-MS/MS:

All labeled peptides mixtures can be separated by online 2D-nanoLC and analysed by electrospray tandem mass spectrometry. The experiments can be carried out on an Eksigent 2D NanoLC Ultra system connected to an LTQ Orbitrap Velos mass spectrometer equipped with a nanoelectrospray ion source (Thermo Electron, Bremen, Germany).

The peptides mixtures can be injected into a 5 cm SCX column (300 μm ID, 5 μm, PolySULFOETHYL Aspartamide column from PolyLC, Columbia, Md.) with a flow of 4 μL/min and eluted in 10 ion exchange elution segments into a C18 trap column (2.5 cm, 100 μm ID, 5 μm, 300 Å ProteoPep II from New Objective, Woburn, Mass.) and washed for 5 min with H2O/0.1% FA. The separation then can be further carried out at 300 nL/min using a gradient of 2-45% B (H2O/0.1% FA (solvent A) and ACN/0.1% FA (solvent B)) for 120 minutes on a 15 cm fused silica column (75 μm ID, 5 μm, 300 Å ProteoPep II from New Objective, Woburn, Mass.).

Full scan MS spectra (m/z 300-2000) can be acquired in the Orbitrap with resolution of 30,000. The most intense ions (up to 10) can be sequentially isolated for fragmentation using High energy C-trap Dissociation (HCD) and dynamically exclude for 30 seconds. HCD can be conducted with an isolation width of 1.2 Da. The resulting fragment ions can be scanned in the orbitrap with resolution of 7500. The LTQ Orbitrap Velos can be controlled by Xcalibur 2.1 with foundation 1.0.1.

Peptides/Proteins Identification and Quantification:

Peptides and proteins can be identified by automated database searching using Proteome Discoverer software (Thermo Electron) with Mascot search engine against SwissProt database. Search parameters can include 10 ppm for MS tolerance, 0.02 Da for MS2 tolerance, and full trypsin digestion allowing for up to 2 missed cleavages. Carbamidomethylation (C) can be set as the fixed modification. Oxidation (M), TMT6, and deamidation (NQ) can be set as dynamic modifications. Peptides and protein identifications can be filtered with Mascot Significant Threshold (p<0.05). The filters can be allowed a 99% confidence level of protein identification (1% FDA).

The Proteome Discoverer software can apply correction factors on the reporter ions, and can reject all quantitation values if not all quantitation channels are present. Relative protein quantitation can be achieved by normalization at the mean intensity.

With respect to the second type of data, in some exemplary embodiments, bioenergetics profiling of pervasive developmental disorder and normal models may employ the Seahorse™ XF24 analyzer to enable the understanding of glycolysis and oxidative phosphorylation components.

Specifically, cells can be plated on Seahorse culture plates at optimal densities. These cells can be plated in 100 μl of media or treatment and left in a 37° C. incubator with 5% CO₂. Two hours later, when the cells are adhered to the 24 well plate, an additional 150 μl of either media or treatment solution can be added and the plates can be left in the culture incubator overnight. This two step seeding procedure allows for even distribution of cells in the culture plate. Seahorse cartridges that contain the oxygen and pH sensor can be hydrated overnight in the calibrating fluid in a non-CO₂ incubator at 37° C. Three mitochondrial drugs are typically loaded onto three ports in the cartridge. Oligomycin, a complex III inhibitor, FCCP, an uncoupler and Rotenone, a complex I inhibitor can be loaded into ports A, B and C respectively of the cartridge. All stock drugs can be prepared at a 10× concentration in an unbuffered DMEM media. The cartridges can be first incubated with the mitochondrial compounds in a non-CO₂ incubator for about 15 minutes prior to the assay. Seahorse culture plates can be washed in DMEM based unbuffered media that contains glucose at a concentration found in the normal growth media. The cells can be layered with 630 ul of the unbuffered media and can be equilibrated in a non-CO₂ incubator before placing in the Seahorse instrument with a precalibrated cartridge. The instrument can be run for three-four loops with a mix, wait and measure cycle for get a baseline, before injection of drugs through the port is initiated. There can be two loops before the next drug is introduced.

OCR (Oxygen consumption rate) and ECAR (Extracellular Acidification Rate) can be recorded by the electrodes in a 7 μl chamber and can be created with the cartridge pushing against the seahorse culture plate.

C. Data Integration and in Silico Model Generation

Once relevant data sets have been obtained, integration of data sets and generation of computer-implemented statistical models may be performed using an AI-based informatics system or platform (e.g, the REFS™ platform). For example, an exemplary AI-based system may produce simulation-based networks of protein associations as key drivers of metabolic end points (ECAR/OCR). See FIG. 4. Some background details regarding the REFS™ system may be found in Xing et al., “Causal Modeling Using Network Ensemble Simulations of Genetic and Gene Expression Data Predicts Genes Involved in Rheumatoid Arthritis,” PLoS Computational Biology, vol. 7, issue. 3, 1-19 (March 2011) (e100105) and U.S. Pat. No. 7,512,497 to Periwal, the entire contents of each of which is expressly incorporated herein by reference in its entirety. In essence, as described earlier, the REFS™ system is an AI-based system that employs mathematical algorithms to establish causal relationships among the input variables (e.g., protein expression levels, mRNA expression levels, and the corresponding functional data, such as the OCR/ECAR values measured on Seahorse culture plates). This process is based only on the input data alone, without taking into consideration prior existing knowledge about any potential, established, and/or verified biological relationships.

In particular, a significant advantage of the platform of the invention is that the AI-based system is based on the data sets obtained from the cell model, without resorting to or taking into consideration any existing knowledge in the art concerning the biological process. Further, preferably, no data points are statistically or artificially cut-off and, instead, all obtained data is fed into the AI-system for determining protein associations. Accordingly, the resulting statistical models generated from the platform are unbiased, since they do not take into consideration any known biological relationships.

Specifically, data from the proteomics and ECAR/OCR can be input into the AI-based information system, which builds statistical models based on data associations, as described above. Simulation-based networks of protein associations are then derived for each disease versus normal scenario, including treatments and conditions using the following methods.

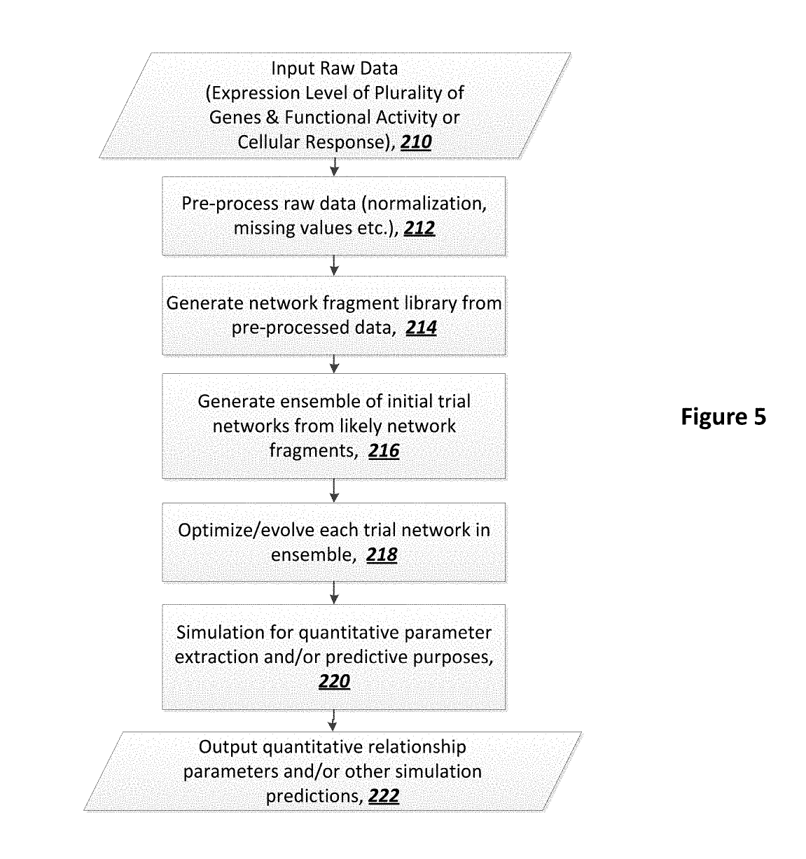

A detailed description of an exemplary process for building the generated (e.g., optimized or evolved) networks appears below with respect to FIG. 5. As described above, data from the proteomics and, optionally, functional cell data is input into the AI-based system (step 210). The input data, which may be raw data or minimally processed data, is pre-processed, which may include normalization (e.g., using a quantile function or internal standards) (step 212). The pre-processing may also include imputing missing data values (e.g., by using the K-nearest neighbor (K-NN) algorithm) (step 212).

The pre-processed data is used to construct a network fragment library (step 214). The network fragments define quantitative, continuous relationships among all possible small sets (e.g., 2-3 member sets or 2-4 member sets) of measured variables (input data). The relationships between the variables in a fragment may be linear, logistic, multinomial, dominant or recessive homozygous, etc. The relationship in each fragment is assigned a Bayesian probabilistic score that reflect how likely the candidate relationship is given the input data, and also penalizes the relationship for its mathematical complexity. By scoring all of the possible pairwise and three-way relationships (and in some embodiments also four-way relationships) inferred from the input data, the most likely fragments in the library can be identified (the likely fragments). Quantitative parameters of the relationship are also computed based on the input data and stored for each fragment. Various model types may be used in fragment enumeration including but not limited to linear regression, logistic regression, (Analysis of Variance) ANOVA models, (Analysis of Covariance) ANCOVA models, non-linear/polynomial regression models and even non-parametric regression. The prior assumptions on model parameters may assume Gull distributions or Bayesian Information Criterion (BIC) penalties related to the number of parameters used in the model. In a network inference process, each network in an ensemble of initial trial networks is constructed from a subset of fragments in the fragment library. Each initial trial network in the ensemble of initial trial networks is constructed with a different subset of the fragments from the fragment library (step 216).

An overview of the mathematical representations underlying the Bayesian networks and network fragments, which is based on Xing et al., “Causal Modeling Using Network Ensemble Simulations of Genetic and Gene Expression Data Predicts Genes Involved in Rheumatoid Arthritis,” PLoS Computational Biology, vol. 7, issue. 3, 1-19 (March 2011) (e100105), is presented below.

A multivariate system with random variables X=X₁, . . . , Xₙ may be characterized by a multivariate probability distribution function P(X₁, . . . , Xₙ; Θ), that includes a large number of parameters Θ. The multivariate probability distribution function may be factorized and represented by a product of local conditional probability distributions:

$$P ( {X}_{1} , … , {X}_{n} ; Θ ) = {\prod }_{i - 1}^{n} {P}_{i} ( {X}_{i} \vert {Y}_{j 1} , … , {Y}_{{jK}_{i}} ; {Θ}_{i} ) ,$$

in which each variable Xᵢ is independent from its non-descendent variables given its Kᵢ parent variables, which are Yⱼ₁, . . . , Y_{jK}_{ᵢ}. After factorization, each local probability distribution has its own parameters Θᵢ.

The multivariate probability distribution function may be factorized in different ways with each particular factorization and corresponding parameters being a distinct probabilistic model. Each particular factorization (model) can be represented by a Directed Acrylic Graph (DAC) having a vertex for each variable Xᵢ and directed edges between vertices representing dependences between variables in the local conditional distributions Pᵢ(Xᵢ|Yⱼ₁, . . . , Y_{jK}_{ᵢ}). Subgraphs of a DAG, each including a vertex and associated directed edges are network fragments.

A model is evolved or optimized by determining the most likely factorization and the most likely parameters given the input data. This may be described as “learning a Bayesian network,” or, in other words, given a training set of input data, finding a network that best matches the input data. This is accomplished by using a scoring function that evaluates each network with respect to the input data.

A Bayesian framework is used to determine the likelihood of a factorization given the input data. Bayes Law states that the posterior probability, P(D|M), of a model M, given data D is proportional to the product of the product of the posterior probability of the data given the model assumptions, P(D|M), multiplied by the prior probability of the model, P(M), assuming that the probability of the data, P(D), is constant across models. This is expressed in the following equation:

$$P ( M \vert D ) = \frac{P ( D \vert M ) * P ( M )}{P ( D )} .$$

The posterior probability of the data assuming the model is the integral of the data likelihood over the prior distribution of parameters:

P(D|M)=∫P(D|M(Θ))P(Θ|M)dΘ.

Assuming all models are equally likely (i.e., that P(M) is a constant), the posterior probability of model M given the data D may be factored into the product of integrals over parameters for each local network fragment Mᵢ as follows:

$$P ( M \vert D ) = {\prod }_{i = 1}^{n} \int {P}_{i} ( {X}_{i} \vert {Y}_{j 1} , … , {Y}_{{jK}_{i}} ; {Θ}_{i} ) .$$

Note that in the equation above, a leading constant term has been omitted. In some embodiments, a Bayesian Information Criterion (BIC), which takes a negative logarithm of the posterior probability of the model P(D|M) may be used to “Score” each model as follows:

$${S}_{tot} ( M ) = - log P ( M \vert D ) = {\sum }_{i = 1}^{n} S ( {M}_{i} ) ,$$

where the total score Sₜₒₜ for a model M is a sum of the local scores Sᵢ for each local network fragment. The BIC further gives an expression for determining a score each individual network fragment:

$$S ( {M}_{i} ) \approx {S}_{BIC} ( {M}_{i} ) = {S}_{MLE} ( {M}_{i} ) + \frac{κ ( {M}_{i} )}{2} log N$$

where κ(Mᵢ) is the number of fitting parameter in model Mᵢ and N is the number of samples (data points). S_{MLE}(Mᵢ) is the negative logarithm of the likelihood function for a network fragment, which may be calculated from the functional relationships used for each network fragment. For a BIC score, the lower the score, the more likely a model fits the input data.

The ensemble of trial networks is globally optimized, which may be described as optimizing or evolving the networks (step 218). For example, the trial networks may be evolved and optimized according to a Metropolis Monte Carlo Sampling algorithm. Simulated annealing may be used to optimize or evolve each trial network in the ensemble through local transformations. In an example simulated annealing processes, each trial network is changed by adding a network fragment from the library, by deleted a network fragment from the trial network, by substituting a network fragment or by otherwise changing network topology, and then a new score for the network is calculated. Generally speaking, if the score improves, the change is kept and if the score worsens the change is rejected. A “temperature” parameter allows some local changes which worsen the score to be kept, which aids the optimization process in avoiding some local minima. The “temperature” parameter is decreased over time to allow the optimization/evolution process to converge.

All or part of the network inference process may be conducted in parallel for the trial different networks. Each network may be optimized in parallel on a separate processor and/or on a separate computing device. In some embodiments, the optimization process may be conducted on a supercomputer incorporating hundreds to thousands of processors which operate in parallel. Information may be shared among the optimization processes conducted on parallel processors.

The optimization process may include a network filter that drops any networks from the ensemble that fail to meet a threshold standard for overall score. The dropped network may be replaced by a new initial network. Further any networks that are not “scale free” may be dropped from the ensemble. After the ensemble of networks has been optimized or evolved, the result may be termed an ensemble of generated cell model networks, which may be collectively referred to as the generated consensus network.

D. Simulation to Extract Quantitative Relationship Information and for Prediction

Simulation may be used to extract quantitative parameter information regarding each relationship in the generated cell model networks (step 220). For example, the simulation for quantitative information extraction may involve perturbing (increasing or decreasing) each node in the network by 10 fold and calculating the posterior distributions for the other nodes (e.g., proteins) in the models. The endpoints are compared by t-test with the assumption of 100 samples per group and the 0.01 significance cut-off. The t-test statistic is the median of 100 t-tests. Through use of this simulation technique, an AUC (area under the curve) representing the strength of prediction and fold change representing the in silico magnitude of a node driving an end point are generated for each relationship in the ensemble of networks.

A relationship quantification module of a local computer system may be employed to direct the AI-based system to perform the perturbations and to extract the AUC information and fold information. The extracted quantitative information may include fold change and AUC for each edge connecting a parent note to a child node. In some embodiments, a custom-built R program may be used to extract the quantitative information.

In some embodiments, the ensemble of generated cell model networks can be used through simulation to predict responses to changes in conditions, which may be later verified though wet-lab cell-based, or animal-based, experiments.

The output of the AI-based system may be quantitative relationship parameters and/or other simulation predictions (222).

E. Generation of Differential (Delta) Networks

A differential network creation module may be used to generate differential (delta) networks between generated cell model networks and generated comparison cell model networks (e.g., a differential (delta) network between a network generated from cells associated with a pervasive developmental disorder, and a network generated from control cells). As described above, in some embodiments, the differential network compares all of the quantitative parameters of the relationships in the generated cell model networks and the generated comparison cell model network. The quantitative parameters for each relationship in the differential network are based on the comparison. In some embodiments, a differential may be performed between various differential networks, which may be termed a delta-delta network. The differential network creation module may be a program or script written in PERL.

F. Visualization of Networks

The relationship values for the ensemble of networks and for the differential networks may be visualized using a network visualization program (e.g., Cytoscape open source platform for complex network analysis and visualization from the Cytoscape consortium). In the visual depictions of the networks, the thickness of each edge (e.g., each line connecting the proteins) represents the strength of fold change. The edges are also directional indicating causality, and each edge has an associated prediction confidence level.

G. Exemplary Computer System

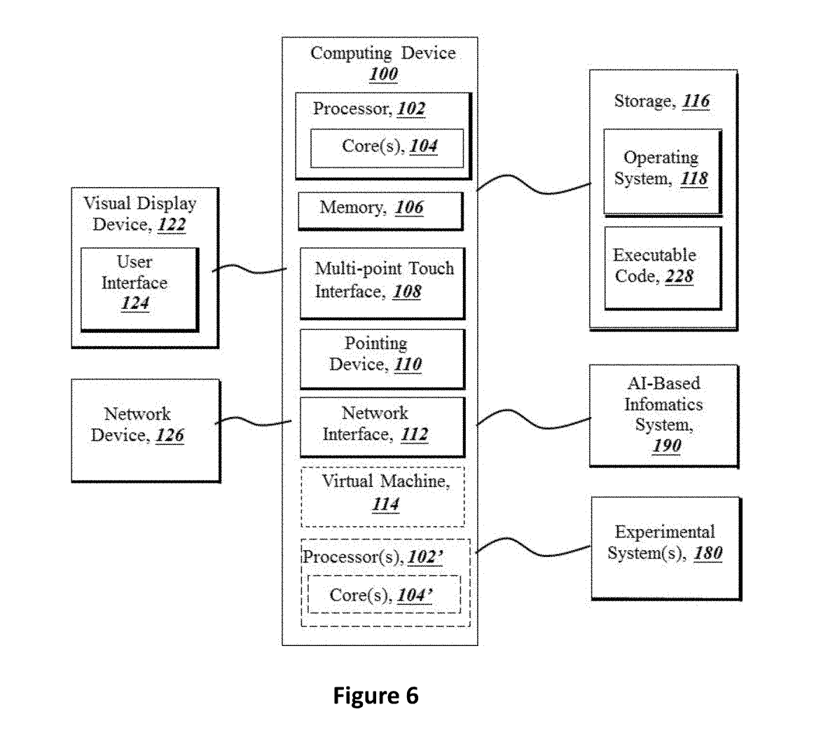

FIG. 6 schematically depicts an exemplary computer system/environment that may be employed in some embodiments for communicating with the AI-based informatics system, for generating differential networks, for visualizing networks, for saving and storing data, and/or for interacting with a user. As explained above, calculations for an AI-based informatics system may be performed on a separate supercomputer with hundreds or thousands of parallel processors that interacts, directly or indirectly, with the exemplary computer system. The environment includes a computing device 100 with associated peripheral devices. Computing device 100 is programmable to implement executable code 150 for performing various methods, or portions of methods, taught herein. Computing device 100 includes a storage device 116, such as a hard-drive, CD-ROM, or other non-transitory computer readable media. Storage device 116 may store an operating system 118 and other related software. Computing device 100 may further include memory 106. Memory 106 may comprise a computer system memory or random access memory, such as DRAM, SRAM, EDO RAM, etc. Memory 106 may comprise other types of memory as well, or combinations thereof. Computing device 100 may store, in storage device 116 and/or memory 106, instructions for implementing and processing each portion of the executable code 150.

The executable code 150 may include code for communicating with the AI-based informatics system 190, for generating differential networks (e.g., a differential network creation module), for extracting quantitative relationship information from the AI-based informatics system (e.g., a relationship quantification module) and for visualizing networks (e.g., Cytoscape).

In some embodiments, the computing device 100 may communicate directly or indirectly with the AI-based informatics system 190 (e.g., a system for executing REFS). For example, the computing device 100 may communicate with the AI-based informatics system 190 by transferring data files (e.g., data frames) to the AI-based informatics system 190 through a network. Further, the computing device 100 may have executable code 150 that provides an interface and instructions to the AI-based informatics system 190.

In some embodiments, the computing device 100 may communicate directly or indirectly with one or more experimental systems 180 that provide data for the input data set. Experimental systems 180 for generating data may include systems for mass spectrometry based proteomics, microarray gene expression, qPCR gene expression, mass spectrometry based metabolomics, and mass spectrometry based lipidomics, SNP microarrays, a panel of functional assays, and other in-vitro biology platforms and technologies.

Computing device 100 also includes processor 102, and may include one or more additional processor(s) 102′, for executing software stored in the memory 106 and other programs for controlling system hardware, peripheral devices and/or peripheral hardware. Processor 102 and processor(s) 102′ each can be a single core processor or multiple core (104 and 104′) processor. Virtualization may be employed in computing device 100 so that infrastructure and resources in the computing device can be shared dynamically. Virtualized processors may also be used with executable code 150 and other software in storage device 116. A virtual machine 114 may be provided to handle a process running on multiple processors so that the process appears to be using only one computing resource rather than multiple. Multiple virtual machines can also be used with one processor.

A user may interact with computing device 100 through a visual display device 122, such as a computer monitor, which may display a user interface 124 or any other interface. The user interface 124 of the display device 122 may be used to display raw data, visual representations of networks, etc. The visual display device 122 may also display other aspects or elements of exemplary embodiments (e.g., an icon for storage device 116). Computing device 100 may include other I/O devices such a keyboard or a multi-point touch interface (e.g., a touchscreen) 108 and a pointing device 110, (e.g., a mouse, trackball and/or trackpad) for receiving input from a user. The keyboard 108 and the pointing device 110 may be connected to the visual display device 122 and/or to the computing device 100 via a wired and/or a wireless connection.

Computing device 100 may include a network interface 112 to interface with a network device 126 via a Local Area Network (LAN), Wide Area Network (WAN) or the Internet through a variety of connections including, but not limited to, standard telephone lines, LAN or WAN links (e.g., 802.11, T1, T3, 56 kb, X.25), broadband connections (e.g., ISDN, Frame Relay, ATM), wireless connections, controller area network (CAN), or some combination of any or all of the above. The network interface 112 may comprise a built-in network adapter, network interface card, PCMCIA network card, card bus network adapter, wireless network adapter, USB network adapter, modem or any other device suitable for enabling computing device 100 to interface with any type of network capable of communication and performing the operations described herein.

Moreover, computing device 100 may be any computer system such as a workstation, desktop computer, server, laptop, handheld computer or other form of computing or telecommunications device that is capable of communication and that has sufficient processor power and memory capacity to perform the operations described herein.

Computing device 100 can be running any operating system 118 such as any of the versions of the MICROSOFT WINDOWS operating systems, the different releases of the Unix and Linux operating systems, any version of the MACOS for Macintosh computers, any embedded operating system, any real-time operating system, any open source operating system, any proprietary operating system, any operating systems for mobile computing devices, or any other operating system capable of running on the computing device and performing the operations described herein. The operating system may be running in native mode or emulated mode.

H. Exemplary Cell Model and Protein Analysis Used to Identify Proteins as Therapeutic Targets and/or Diagnostic Markers for Pervasive Developmental Disorder

Virtually all disease conditions involve complicated interactions among different cell types and/or organ systems. Perturbation of critical functions in one cell type or organ may lead to secondary effects on other interacting cells types and organs, and such downstream changes may in turn feedback to the initial changes and cause further complications.

Therefore, it may be beneficial to dissect a given disease condition to its components, such as interaction between pairs of cell types or organs, and systemically probe the interactions between these components in order to gain a more complete, global view of the disease condition.

To this end, Applicants have identified multiple sets of cell pairs for use in the subject discovery platform in a number of disease conditions relating to pervasive developmental disorder, such as autism and Alzheimer's disease, and have conducted experiments using the discovery platform to decipher the critical determinative differentials that may be important for the particular disease status. Cell lines indicated below have been processed and analyzed as described herein.

| Cell line 1 | Cell line 2 | Disease model |
|---|---|---|
| Cells from Autistic | Cell line from control, | Autism |
| Individual | healthy individual (e.g., | |
| | sibling or parent who is not | |
| | afflicted with Autism) | |
| Cell line from Individual | Cell line from control, | Alzheimer's |
| afflicted with Alzheimer's | healthy individual (e.g., | disease |
| disease | sibling or parent who is not | |
| | afflicted with Alzheimer's | |
| | disease) | |

Various stress conditions/stressors may be employed in each of the listed disease conditions. These stressors/conditions may constitute the external stimulus for the cell systems. For example, the cells may be treated with Coenzyme Q10.

### 1. Proteomic Sample Analysis

In certain embodiments, the subject method employs large-scale high-throughput quantitative proteomic analysis of hundreds of samples of similar character, and provide the data necessary for identifying the cellular output differentials.

There are numerous art-recognized technologies suitable for this purpose. An exemplary technique, iTRAQ analysis in combination with mass spectrometry, is briefly described below.

To provide reference samples for relative quantification with the iTRAQ technique, multiple QC pools are created. Two separate QC pools, consisting of aliquots of each sample, were generated from the Cell #1 and Cell #2 samples—these samples are denoted as QCS1 and QCS2, and QCP1 and QCP2 for supernatants and pellets, respectively. In order to allow for protein concentration comparison across the two cell lines, cell pellet aliquots from the QC pools described above are combined in equal volumes to generate reference samples (QCP).

The quantitative proteomics approach is based on stable isotope labeling with the 8-plex iTRAQ reagent and 2D-LC MALDI MS/MS for peptide identification and quantification. Quantification with this technique is relative: peptides and proteins are assigned abundance ratios relative to a reference sample. Common reference samples in multiple iTRAQ experiments facilitate the comparison of samples across multiple iTRAQ experiments.

To implement this analysis scheme, six primary samples and two control pool samples are combined into one 8-plex iTRAQ mix, with the control pool samples labeled with 113 and 117 reagents according to the manufacturer's suggestions. This mixture of eight samples is then fractionated by two-dimensional liquid chromatography; strong cation exchange (SCX) in the first dimension, and reversed-phase HPLC in the second dimension. The HPLC eluent is directly fractionated onto MALDI plates, and the plates are analyzed on an MDS SCIEX/AB 4800 MALDI TOF/TOF mass spectrometer.

In the absence of additional information, it is assumed that the most important changes in protein expression are those within the same cell types under different treatment conditions. For this reason, primary samples from Cell#1 and Cell#2 are analyzed in separate iTRAQ mixes. To facilitate comparison of protein expression in Cell#1 vs. Cell#2 samples, universal QCP samples are analyzed in the available “iTRAQ slots” not occupied by primary or cell line specific QC samples (QC1 and QC2).

A brief overview of the laboratory procedures employed is provided herein.

a. Protein Extraction from Cell Supernatant Samples

For cell supernatant samples (CSN), proteins from the culture medium are present in a large excess over proteins secreted by the cultured cells. In an attempt to reduce this background, upfront abundant protein depletion was implemented. As specific affinity columns are not available for bovine or horse serum proteins, an anti-human IgY14 column was used. While the antibodies are directed against human proteins, the broad specificity provided by the polyclonal nature of the antibodies was anticipated to accomplish depletion of both bovine and equine proteins present in the cell culture media that was used.

A 200-μl aliquot of the CSN QC material is loaded on a 10-mL IgY14 depletion column before the start of the study to determine the total protein concentration (Bicinchoninic acid (BCA) assay) in the flow-through material. The loading volume is then selected to achieve a depleted fraction containing approximately 40 μg total protein.

b. Protein Extraction from Cell Pellets

An aliquot of Cell #1 and Cell #2 is lysed in the “standard” lysis buffer used for the analysis of tissue samples at BGM, and total protein content is determined by the BCA assay. Having established the protein content of these representative cell lystates, all cell pellet samples (including QC samples described in Section 1.1) were processed to cell lysates. Lysate amounts of approximately 40 □g of total protein were carried forward in the processing workflow.

c. Sample Preparation for Mass Spectrometry

Sample preparation follows standard operating procedures and constitute of the following:
Reduction and alkylation of proteins
Protein clean-up on reversed-phase column (cell pellets only)
Digestion with trypsin
iTRAQ labeling
Strong cation exchange chromatography—collection of six fractions (Agilent 1200 system)
HPLC fractionation and spotting to MALDI plates (Dionex Ultimate3000/Probot system)

d. MALDI MS and MS/MS

HPLC-MS generally employs online ESI MS/MS strategies. BG Medicine uses an off-line LC-MALDI MS/MS platform that results in better concordance of observed protein sets across the primary samples without the need of injecting the same sample multiple times. Following first pass data collection across all iTRAQ mixes, since the peptide fractions are retained on the MALDI target plates, the samples can be analyzed a second time using a targeted MS/MS acquisition pattern derived from knowledge gained during the first acquisition. In this manner, maximum observation frequency for all of the identified proteins is accomplished (ideally, every protein should be measured in every iTRAQ mix).

e. Data Processing

The data processing process within the BGM Proteomics workflow can be separated into those procedures such as preliminary peptide identification and quantification that are completed for each iTRAQ mix individually (Section 1.5.1) and those processes (Section 1.5.2) such as final assignment of peptides to proteins and final quantification of proteins, which are not completed until data acquisition is completed for the project.

The main data processing steps within the BGM Proteomics workflow are:
Peptide identification using the Mascot (Matrix Sciences) database search engine
Automated in house validation of Mascot IDs
Quantification of peptides and preliminary quantification of proteins
Expert curation of final dataset
Final assignment of peptides from each mix into a common set of proteins using the automated PVT tool
Outlier elimination and final quantification of proteins

i. Data Processing of Individual iTRAQ Mixes

As each iTRAQ mix is processed through the workflow the MS/MS spectra are analyzed using proprietary BGM software tools for peptide and protein identifications, as well as initial assessment of quantification information. Based on the results of this preliminary analysis, the quality of the workflow for each primary sample in the mix is judged against a set of BGM performance metrics. If a given sample (or mix) does not pass the specified minimal performance metrics, and additional material is available, that sample is repeated in its entirety and it is data from this second implementation of the workflow that is incorporated in the final dataset.

ii. Peptide Identification

MS/MS spectra was searched against the Uniprot protein sequence database containing human, bovine, and horse sequences augmented by common contaminant sequences such as porcine trypsin. The details of the Mascot search parameters, including the complete list of modifications, are given in Table 1.

TABLE 1
Mascot Search Parameters

| Precursor mass tolerance | 100 ppm |
|---|---|
| Fragment mass tolerance | 0.4 Da |
| Variable modifications | N-term iTRAQ8 |
| | Lysine iTRAQ8 |
| | Cys carbamidomethyl |
| | Pyro-Glu (N-term) |
| | Pyro-Carbamidomethyl Cys |
| | (N-term) |
| | Deamidation (N only) |
| | Oxidation (M) |
| Enzyme specificity | Fully Tryptic |
| Number of missed tryptic sites allowed | 2 |
| Peptide rank considered | 1 |

After the Mascot search is complete, an auto-validation procedure is used to promote (i.e., validate) specific Mascot peptide matches. Differentiation between valid and invalid matches is based on the attained Mascot score relative to the expected Mascot score and the difference between the Rank 1 peptides and Rank 2 peptide Mascot scores. The criteria required for validation are somewhat relaxed if the peptide is one of several matched to a single protein in the iTRAQ mix or if the peptide is present in a catalogue of previously validated peptides.

iii. Peptide and Protein Quantification

The set of validated peptides for each mix is utilized to calculate preliminary protein quantification metrics for each mix. Peptide ratios are calculated by dividing the peak area from the iTRAQ label (i.e., m/z 114, 115, 116, 118, 119, or 121) for each validated peptide by the best representation of the peak area of the reference pool (QC1 or QC2). This peak area is the average of the 113 and 117 peaks provided both samples pass QC acceptance criteria. Preliminary protein ratios are determined by calculating the median ratio of all “useful” validated peptides matching to that protein. “Useful” peptides are fully iTRAQ labeled (all N-terminal are labeled with either Lysine or PyroGlu) and fully Cysteine labeled (i.e., all Cys residues are alkylated with Carbamidomethyl or N-terminal Pyro-cmc).

f. Post-Acquisition Processing

Once all passes of MS/MS data acquisition are complete for every mix in the project, the data is collated using the three steps discussed below which are aimed at enabling the results from each primary sample to be simply and meaningfully compared to that of another.

i. Global Assignment of Peptide Sequences to Proteins

Final assignment of peptide sequences to protein accession numbers is carried out through the proprietary Protein Validation Tool (PVT). The PVT procedure determines the best, minimum non-redundant protein set to describe the entire collection of peptides identified in the project. This is an automated procedure that has been optimized to handle data from a homogeneous taxonomy.

Protein assignments for the supernatant experiments were manually curated in order to deal with the complexities of mixed taxonomies in the database. Since the automated paradigm is not valid for cell cultures grown in bovine and horse serum supplemented media, extensive manual curation is necessary to minimize the ambiguity of the source of any given protein.

ii. Normalization of Peptide Ratios

The peptide ratios for each sample are normalized based on the method of Vandesompele et al. Genome Biology, 2002, 3(7), research 0034.1-11. This procedure is applied to the cell pellet measurements only. For the supernatant samples, quantitative data are not normalized considering the largest contribution to peptide identifications coming from the media.

iii. Final Calculation of Protein Ratios

A standard statistical outlier elimination procedure is used to remove outliers from around each protein median ratio, beyond the 1.96σ level in the log-transformed data set. Following this elimination process, the final set of protein ratios are (re-)calculated.

### IV. Pervasive Developmental Disorders

Pervasive developmental disorders are neurodevelopmental disorders that include autistic disorder, Asperger's syndrome, pervasive developmental disorder-not otherwise specified (PDD-NOS), Rett's syndrome, and childhood disintegrative disorder. The disorders and diagnostic criteria are provided in the Diagnostic and Statistical Manual of Mental Disorders, 4^{th} edition (DSM-IV); International Classification of Diseases, 10^{th} edition; Levy et al.), the pertinent contents of which are expressly incorporated herein by reference. Autism spectrum disorders include autistic disorder (also known autism), Asperger's syndrome, and PDD-NOS. Autism spectrum disorders are observed three to four times more frequently in males than in females. In the U.S.A. and Europe, prevalence rates of autism spectrum disorders have increased dramatically since the 1960s. Prevalence rates are estimated at about 1 in 150.

Autism spectrum disorders are characterized by qualitative impairments in social functioning and communication, often accompanied by repetitive and stereotyped patterns of behavior and interests. Autism or autistic disorder involves a severe and pervasive impairment in reciprocal socialization. Asperger's syndrome differs from other autism spectrum disorders by its relative preservation of linguistic and cognitive development. Although not required for diagnosis, physical clumsiness and atypical use of language are frequently reported in Asperger's syndrome. Pervasive developmental disorder-not otherwise specified (PDD-NOS, also known as “atypical personality development,” “atypical PDD,” or “atypical autism”) is included in DSM-IV to encompass cases where there is marked impairment of social interaction, communication, and/or stereotyped behavior patterns or interest, but full features of another pervasive developmental disorder are not met. Individuals diagnosed with PDD-NOS may have difficulties socializing, exhibit repetitive behaviors, or be oversensitive to certain stimuli. In their interaction with others they may struggle to maintain eye contact, appear unemotional, or appear to be unable to speak. They may also have difficulty transitioning from one activity to another.

Individuals with autism spectrum disorders also exhibit obsessive-compulsive behaviors that partially overlap with symptoms associated with obsessive compulsive disorder. It is contemplated that the methods provided by this invention can be used to treat obsessive compulsive symptoms in individuals with pervasive developmental disorders, as well as other types of disorders such as obsessive compulsive disorder that have similar symptoms or causes.

Autism spectrum disorders are highly heritable; estimates of heritability from family and twin studies suggest that approximately 90% of the variance is attributable to genetic factors. Parents and siblings of those affected often show subsyndromal manifestations of autism (“the broad autism phenotype”), which include delayed language, difficulties with social aspects of language, delayed social development, absence of close friendships, and a perfectionistic or rigid personality style. However, neither the genetic aspects nor the complex etiology of the disorders are not understood.

Rett's syndrome is a neurodevelopmental disorder observed primarily in girls and characterized by small hands and feet, repetitive hand movements, and a deceleration of the rate of head growth. Girls with Rett's syndrome are prone to gastrointestinal disorders, up to 80% have seizures, they typically have no verbal skills, and about 50% are not ambulatory. Scoliosis, growth failure, and constipation are also very common.

Childhood disintegrative disorder (CDD), also known as Heller's syndrome and disintegrative psychosis, is characterized by developmental delays in language, social function, and motor skills that appear from the age of 2 to around the age of 10 years of age. CDD is sometimes considered a low-functioning form of autism.

As used herein, a subject “exhibiting one or more signs or symptoms of a pervasive developmental disorder” includes a subject that suffers from a pervasive developmental disorder, as well as a subject that does not suffer from the developmental disorder but that exhibits subsyndromal manifestations of a pervasive developmental disorder, such as the broad autism phenotype, which is described, for example, in the DSM-IV, in Piven et al. Am J Psychiatry 154: 185-190 (1997) and Losh et al. Am J Med Genet B Neuropsychiatr Genet 147: 424-433 (2008). Identification, quantitation, and/or monitoring of one or more signs or symptoms of a pervasive developmental disorder, particularly autism, can be accomplished using the Autism Diagnostic Observation Schedule (ADOS) (Lord et al., J. Autism Dev Dis. 19:185-212 (1989) incorporated herein by reference) and/or the Revised Autism Diagnostic Interview (ADI-R) (Lord, et al., J. Autism Dev Dis. 24:659-685 (1994). As used herein, one or more signs or symptoms of a pervasive developmental disorder are those signs or symptoms included in the diagnostic criteria for the pervasive developmental disorders and do not include other signs or symptoms commonly observed with pervasive developmental disorder that are not an aspect of the diagnostic criteria e.g., constipation, seizure disorder, mental retardation, physical malformation resulting in delayed speech, etc.

A subject “exhibiting one or more sign or symptoms of a pervasive developmental disorder” also includes a nonhuman subject that exhibits such symptoms. Non-human animals that exhibit signs or symptoms of pervasive developmental disorder include animal models of these disorders. A number of mice having various genetic mutations have been suggested for use as models of autism and other pervasive developmental disorders as discussed herein. Drosophila models of fragile X syndrome are known (as discussed below, fragile X genotype is associated with autism) and as well as mouse models of Rett's syndrome.

A subject that “suffers from” a pervasive developmental disorder includes a subject that has been clinically diagnosed with such a disorder as well as a subject that meets diagnostic criteria for having such a disorder. Diagnostic criteria and methods for diagnosing autism spectrum disorders are discussed in Levy et al and the DSM-IV.

Diagnostic criteria in the DSM-IV for various pervasive developmental disorders are as follows:

299.00 Autistic Disorder
(A) total of six (or more) items from (1), (2), and (3), with at least two from (1), and one each from (2) and (3):
(1) qualitative impairment in social interaction, as manifested by at least two of the following:
(a) marked impairment in the use of multiple nonverbal behaviors such as eye-to-eye gaze, facial expression, body postures, and gestures to regulate social interaction
(b) failure to develop peer relationships appropriate to developmental level
(c) a lack of spontaneous seeking to share enjoyment, interests, or achievements with other people (e.g., by a lack of showing, bringing, or pointing out objects of interest)
(d) lack of social or emotional reciprocity
(2) qualitative impairments in communication as manifested by at least one of the following:
(a) delay in, or total lack of, the development of spoken language (not accompanied by an attempt to compensate through alternative modes of communication such as gestures or mime)
(b) in individuals with adequate speech, marked impairment in the ability to initiate or sustain a conversation with others
(c) stereotyped and repetitive use of language or idiosyncratic language
(d) lack of varied, spontaneous make-believe play or social imitative play appropriate to developmental level
(3) restricted repetitive and stereotyped patterns of behavior, interests, and activities, as manifested by at least one of the following:
(a) encompassing preoccupation with one or more stereotyped patterns of interest that is abnormal either in intensity or focus
(b) apparently inflexible adherence to specific, nonfunctional routines or rituals
(c) stereotyped and repetitive motor mannerisms (e.g., hand or finger flapping or twisting, or complex whole-body movements)
(d) persistent preoccupation with parts of objects
(B) Delays or abnormal functioning in at least one of the following areas, with onset prior to age 3 years: (1) social interaction, (2) language as used in social communication, or (3) symbolic or imaginative play.
(C) The disturbance is not better accounted for by Rett's Disorder or Childhood Disintegrative Disorder.

299.80 Rett's Disorder
(A) All of the following:
(1) apparently normal prenatal and perinatal development
(2) apparently normal psychomotor development through the first 5 months after birth
(3) normal head circumference at birth
(B) Onset of all of the following after the period of normal development:
(1) deceleration of head growth between ages 5 and 48 months
(2) loss of previously acquired purposeful hand skills between ages 5 and 30 months with the subsequent development of stereotyped hand movements (e.g., hand-wringing or hand washing)
(3) loss of social engagement early in the course (although often social interaction develops later)
(4) appearance of poorly coordinated gait or trunk movements
(5) severely impaired expressive and receptive language development with severe psychomotor retardation

299.10 Childhood Disintegrative Disorder
(A) Apparently normal development for at least the first 2 years after birth as manifested by the presence of age-appropriate verbal and nonverbal communication, social relationships, play, and adaptive behavior.
(B) Clinically significant loss of previously acquired skills (before age 10 years) in at least two of the following areas:
(1) expressive or receptive language
(2) social skills or adaptive behavior
(3) bowel or bladder control
(4) play
(5) motor skills
(C) Abnormalities of functioning in at least two of the following areas:
(1) qualitative impairment in social interaction (e.g., impairment in nonverbal behaviors, failure to develop peer relationships, lack of social or emotional reciprocity)
(2) qualitative impairments in communication (e.g., delay or lack of spoken language, inability to initiate or sustain a conversation, stereotyped and repetitive use of language, lack of varied make-believe play)
(3) restricted, repetitive, and stereotyped patterns of behavior, interests, and activities, including motor stereotypes and mannerisms
(D) The disturbance is not better accounted for by another specific Pervasive Developmental Disorder or by Schizophrenia.

299.80 Asperger's Disorder
(A) Qualitative impairment in social interaction, as manifested by at least two of the following:
(1) marked impairment in the use of multiple nonverbal behaviors such as eye-to-eye gaze, facial expression, body postures, and gestures to regulate social interaction
(2) failure to develop peer relationships appropriate to developmental level
(3) a lack of spontaneous seeking to share enjoyment, interests, or achievements with other people (e.g., by a lack of showing, bringing, or pointing out objects of interest to other people) lack of social or emotional reciprocity.
(B) Restricted repetitive and stereotyped patterns of behavior, interests, and activities, as manifested by at least one of the following:
(1) encompassing preoccupation with one or more stereotyped and restricted patterns of interest that is abnormal either in intensity or focus
(2) apparently inflexible adherence to specific, non-functional routines or rituals
(3) stereotyped and repetitive motor mannerisms (e.g., hand or finger flapping or twisting, or complex whole-body movements)
(4) persistent preoccupation with parts of objects
(C) The disturbance causes clinically significant impairment in social, occupational, or other important areas of functioning.
(D) There is no clinically significant general delay in language (e.g., single words used by age 2 years, communicative phrases used by age 3 years)
(E) There is no clinically significant delay in cognitive development or in the development of age-appropriate self-help skills, adaptive behavior (other than in social interaction), and curiosity about the environment in childhood.
(F) Criteria are not met for another specific Pervasive Developmental Disorder or Schizophrenia.

299.80 Pervasive Developmental Disorder not Otherwise Specified (Including Atypical Autism)
This category should be used when there is a severe and pervasive impairment in the development of reciprocal social interaction or verbal and nonverbal communication skills, or when stereotyped behavior, interests, and activities are present, but the criteria are not met for a specific Pervasive Developmental Disorder, Schizophrenia, Schizotypal Personality Disorder, or Avoidant Personality Disorder. For example, this category includes atypical autism—presentations that do not meet the criteria for Autistic Disorder because of late age of onset, atypical symptomatology, or subthreshold symptomatology, or all of these.

Genetics of Autism and Pervasive Developmental Disorders

Autism is considered to be a complex multifactorial disorder involving many genes. Accordingly, several loci have been identified, some or all of which may contribute to the phenotype. Included in this entry is AUTS1, which has been mapped to chromosome 7q22.

Other susceptibility loci include AUTS3 (608049), which maps to chromosome 13q14; AUTS4 (608636), which maps to chromosome 15q11; AUTS5 (606053), which maps to chromosome 2q; AUTS6 (609378), which maps to chromosome 17q11; AUTS7 (610676), which maps to chromosome 17q21; AUTS8 (607373), which maps to chromosome 3q25-q27; AUTS9 (611015), which maps to chromosome 7q31; AUTS10 (611016), which maps to chromosome 7q36; AUTS11 (610836), which maps to chromosome 1q41; AUTS12 (610838), which maps to chromosome 21p13-q11; AUTS13 (610908), which maps to chromosome 12q14; AUTS14 (611913), which maps to chromosome 16p11.2; AUTS15 (612100), associated with mutation in the CNTNAP2 gene (604569) on chromosome 7q35-q36; AUTS16 (613410), associated with mutation in the SLC9A9 gene (608396) on chromosome 3q24; and AUTS17 (613436), associated with mutation in the SHANK2 gene (603290) on chromosome 11q13. (NOTE: the symbol ‘AUTS2’ has been used to refer to a gene on chromosome 7q11 (KIAA0442; 607270) and therefore is not used as a part of this autism locus series.)

Three X-linked forms of autism (AUTSX1; 300425; AUTSX2; 300495; AUTSX3; 300496) are associated with mutations in the NLGN3 (300336), NLGN4 (300427), and MECP2 (300005) genes, respectively.

In addition to mapping studies, functional candidate gene and proteomic approaches have identified variants in specific genes that may affect susceptibility to the development of autism; see, e.g., the glyoxalase I gene (GLO1; 138750) on chromosome 6p21.3.

Animal Models of Pervasive Developmental Disorders

A number of mouse models have been suggested as possibly being relevant for use as models for autism or pervasive developmental disorders. The following are provided as examples of animal models that can be used to study the efficacy and safety of a therapeutic agent, e.g., the proteins listed in Tables 2-6. It is understood that additional animal models are available and will become available in the future that can be used in relation to the instant invention. Most of the mice are commercially available, e.g., from Jackson Laboratories in Bar Harbor, Me. (see, e.g., Mice strain sheds new light on autism JAX® NOTES Issue 512, Winter 2008).

The neuroligin3 knock out mouse is a targeted mutation strain carries a deletion of exons 2 and 3 of the gene (B6;129-Nlgn3^{tm2.1Sud}/J (Tabuchi et al., Science 318(5847):71-6 (2007)). These mice show no alteration in their inhibitory synaptic transmission characteristics. Homozygotes are viable, normal in size and do not display any gross physical abnormalities. It has been suggested that this mutant mouse strain may be useful in studies of synapse formation and/or function and neurodevelopmental defects, such as autism. A second neuroligin3 transgenic mouse was generated with an R451C mutation in exon 7 which is flanked by loxP sites B6;129-Nlgn3^{tm1Sud}/J). Mutant mice exhibit enhancements in inhibitory synaptic transmission as well as spacial learning and memory, but show deficits in social interaction. It has been suggested that this mutant mouse strain may be useful in studies of the pathophysiology of autism. When used in conjunction with a Cre recombinase-expressing strain, this strain is useful in generating tissue-specific mutants of the foxed allele. Mice that are homozygous for the targeted mutation are viable, fertile, normal in size and do not display any gross physical abnormalities.

A transgenic mouse overexpressing rat neuroligin 2 (B6.Cg-Tg(Thy1-Nlgn2)6Hnes/J) has been suggested as a model for autism and Rett's syndrome (Hines et al., J Neurosci 28:6055-67, 2008). Mice hemizygous for the TgNL2 transgene are viable and fertile, but hemizygous females are poor mothers. The TgNL2 transgene encodes a hemagglutinin-tagged rat neuroligin 2 (Nlgn2 or NL2) gene driven by the murine Thy1.2 expression cassette. HA-NL2 transcript and protein is expressed throughout the neuroaxis in neuronal cells (high levels in cortex and limbic structures such as amygdala and hippocampus) and is predominantly localized to inhibitory synaptic contacts. TgNL2.6 mice have moderate to high levels of HA-NL2 expression (approximately 1.6-fold greater than wild type NL2). This overexpression leads to reduced lifespan and body weight, and induces aberrant synapse maturation and altered neuronal excitability that lead to behavioral deficits. Specifically, TgNL2.6 mice manifest disorders reminiscent of autism and/or Rett syndrome; jumping, limb clasping, anxiety, and impaired social interactions. Transgenic mice also exhibit Straub tail, transient episodes of kyphosis, and enhanced incidence of spike-wave discharges.

Mice with aberrant expression of beta3 coding region of the Gabrb3 (gamma-aminobutyric acid (GABA-A) receptor, subunit beta 3) have been suggested for use as a model for autism spectrum disorder (129-Gabrb3^{tm1Geh}/J) (Delorey et al., Behav Brain Res 187:207-20, 2008; Homanics et al., Proc Natl Acad Sci USA 94:4143-8, 1997). The mice demonstrate multiple phenotypic abnormalities including cleft palate, seizures, epilepsy, and sensitivity to anesthetics and ethanol. In addition, the observed behavioral deficits (especially regarding social behaviors) indicate that mutant mice may be a useful model of autism spectrum disorders.

The BTBR T⁺ tf/J are a spontaneously occurring mutant mouse strain including mutations in at least the tufted (tf) gene and the Disci gene (Petkov et al., Genomics 83:902-11, 2004) which is known to be involved in schizophrenia. The mice exhibit a 100% absence of the corpus callosum and a severely reduced hippocampal commissure (Wahlsten D, 2003 Brain Res. 971:47-54). This strain exhibits several symptoms of autism including: reduced social interactions, impaired play, low exploratory behavior, unusual vocalizations and high anxiety as compared to other inbred strains (McFarlane et al., Gen, Brain Behav 7:152-63, 2008; Moy et al., Behav Br Res. 176:4-20, 2007; Scattoni et al., PLoS ONE, 3:e3067, 2008).

Mice with a mutation in the arginine vasopressin receptor 1B was generated by replacing the coding region from before the initiating methionine to just upstream of the transmembrane VI region of the endogenous gene with a neomycin resistance cassette. The mice have been suggested to be useful in studies of aggressive behavior, social motivation, and appropriate behavioral responses, and may be potential models of autism and aggression accompanying dementia and traumatic brain injury (B6;129X1-Avpr1b^{tm1Wsy}/J). Mice homozygous for this targeted mutation are viable, fertile, normal in size, exhibit apparently normal sexual behavior, and do not display any gross physical abnormalities. Homozygous mice have been demonstrated to exhibit less social aggression, altered chemoinvestigatory behavior, and impaired social recognition (Wersinger et al., Horm Behav 46:638-45, 2004).

Other mice useful as models for autism or other pervasive developmental disorders can be found using the database at jaxmice.jax.org/query/f?p=205:1:2176162254083441.

### V. Markers of the Invention

The invention relates to markers (hereinafter “biomarkers”, “markers” or “markers of the invention”). Preferred markers of the invention are the markers listed in Tables 2-6.

The invention provides nucleic acids and proteins that are encoded by or correspond to the markers (hereinafter “marker nucleic acids” and “marker proteins,” respectively). These markers are particularly useful in screening for the presence of a pervasive developmental disorder, in assessing severity of a pervasive developmental disorder, assessing whether a subject is afflicted with a pervasive developmental disorder, identifying a composition for treating a pervasive developmental disorder, assessing the efficacy of an environmental influencer compound for treating a pervasive developmental disorder, monitoring the progression of a pervasive developmental disorder, prognosing the aggressiveness of a pervasive developmental disorder, prognosing the survival of a subject with a pervasive developmental disorder, prognosing the recurrence of a pervasive developmental disorder and prognosing whether a subject is predisposed to developing a pervasive developmental disorder.

In some embodiments of the present invention, one or more biomarkers is used in connection with the methods of the present invention. As used herein, the term “one or more biomarkers” is intended to mean that at least one biomarker in a disclosed list of biomarkers is assayed and, in various embodiments, more than one biomarker set forth in the list may be assayed, such as two, three, four, five, ten, twenty, thirty, forty, fifty, more than fifty, or all the biomarkers in the list may be assayed.

A “marker” is a gene whose altered level of expression in a tissue or cell from its expression level in normal or healthy tissue or cell is associated with a disease state, such as a pervasive developmental disorder (e.g., autism or Alzheimer's disease). A “marker nucleic acid” is a nucleic acid (e.g., mRNA, cDNA) encoded by or corresponding to a marker of the invention. Such marker nucleic acids include DNA (e.g., cDNA) comprising the entire or a partial sequence of any of SEQ ID NO (nts) or the complement of such a sequence. The marker nucleic acids also include RNA comprising the entire or a partial sequence of any SEQ ID NO (nts) or the complement of such a sequence, wherein all thymidine residues are replaced with uridine residues. A “marker protein” is a protein encoded by or corresponding to a marker of the invention. A marker protein comprises the entire or a partial sequence of any of the SEQ ID NO (AAs). The terms “protein” and “polypeptide’ are used interchangeably.

The “normal” level of expression of a marker is the level of expression of the marker in cells of a human subject or patient not afflicted with a pervasive developmental disorder (e.g., autism or Alzheimer's disease).

An “over-expression” or “higher level of expression” of a marker refers to an expression level in a test sample that is greater than the standard error of the assay employed to assess expression, and is preferably at least twice, and more preferably three, four, five, six, seven, eight, nine or ten times the expression level of the marker in a control sample (e.g., sample from a healthy subject not having the marker associated disease, i.e., a pervasive developmental disorder) and preferably, the average expression level of the marker in several control samples.

A “lower level of expression” of a marker refers to an expression level in a test sample that is at least twice, and more preferably three, four, five, six, seven, eight, nine or ten times lower than the expression level of the marker in a control sample (e.g., sample from a healthy subjects not having the marker associated disease, i.e., a pervasive developmental disorder) and preferably, the average expression level of the marker in several control samples.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

“Proteins of the invention” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The invention further provides antibodies, antibody derivatives and antibody fragments which specifically bind with the marker proteins and fragments of the marker proteins of the present invention. Unless otherwise specified herewithin, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In certain embodiments, where a particular listed gene is associated with more than one treatment conditions, such as at different time periods after a treatment, or treatment by different concentrations of a potential environmental influencer, the fold change for that particular gene refers to the longest recorded treatment time. In other embodiments, the fold change for that particular gene refers to the shortest recorded treatment time. In other embodiments, the fold change for that particular gene refers to treatment by the highest concentration of env-influencer. In other embodiments, the fold change for that particular gene refers to treatment by the lowest concentration of env-influencer. In yet other embodiments, the fold change for that particular gene refers to the modulation (e.g., up- or down-regulation) in a manner that is consistent with the therapeutic effect of the env-influencer.

In certain embodiments, the positive or negative fold change refers to that of any gene described herein.

As used herein, “positive fold change” refers to “up-regulation” or “increase (of expression)” of a marker that is listed herein.

As used herein, “negative fold change” refers to “down-regulation” or “decrease (of expression)” of a marker that is listed herein.

Various aspects of the invention are described in further detail in the following subsections.

### 1. Isolated Nucleic Acid Molecules

One aspect of the invention pertains to isolated nucleic acid molecules, including nucleic acids which encode a marker protein or a portion thereof. Isolated nucleic acids of the invention also include nucleic acid molecules sufficient for use as hybridization probes to identify marker nucleic acid molecules, and fragments of marker nucleic acid molecules, e.g., those suitable for use as PCR primers for the amplification or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a marker nucleic acid or to the nucleotide sequence of a nucleic acid encoding a marker protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker nucleic acid or which encodes a marker protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a marker protein (e.g., protein having the sequence of the SEQ ID NO (AAs)), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a marker nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a variant marker protein that contain changes in amino acid residues that are not essential for activity. Such variant marker proteins differ in amino acid sequence from the naturally-occurring marker proteins, yet retain biological activity. In one embodiment, such a variant marker protein has an amino acid sequence that is at least about 40% identical, 50%, 60%, 70%, 80%, 90%, 91%, 92%, 93%, 94%, 95%, 96%, 97%, 98% or 99% identical to the amino acid sequence of a marker protein.

An isolated nucleic acid molecule encoding a variant marker protein can be created by introducing one or more nucleotide substitutions, additions or deletions into the nucleotide sequence of marker nucleic acids, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, is oleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded marker cDNA molecule or complementary to a marker mRNA sequence. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a marker protein. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a pervasive developmental disorder-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEES Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a marker protein can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a marker of the invention can be inhibited by targeting nucleotide sequences complementary to the regulatory region of the gene encoding the marker nucleic acid or protein (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

### 2. Isolated Proteins and Antibodies

One aspect of the invention pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein of the invention can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein.

Preferred marker proteins are encoded by nucleotide sequences comprising the sequence of any of the SEQ ID NO (nts). Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring marker protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins comprising a marker protein or a segment thereof. As used herein, a “chimeric protein” or “fusion protein” comprises all or part (preferably a biologically active part) of a marker protein operably linked to a heterologous polypeptide (i.e., a polypeptide other than the marker protein). Within the fusion protein, the term “operably linked” is intended to indicate that the marker protein or segment thereof and the heterologous polypeptide are fused in-frame to each other. The heterologous polypeptide can be fused to the amino-terminus or the carboxyl-terminus of the marker protein or segment.

One useful fusion protein is a GST fusion protein in which a marker protein or segment is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a marker protein can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, N Y, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a marker protein is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a marker protein. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a marker protein in a subject, to purify ligands and in screening assays to identify molecules which inhibit the interaction of the marker protein with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of marker proteins. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to marker proteins, fusion proteins or segments thereof having a signal sequence, as well as to such proteins from which the signal sequence has been proteolytically cleaved (i.e., the cleavage products). In one embodiment, a nucleic acid sequence encoding a signal sequence can be operably linked in an expression vector to a protein of interest, such as a marker protein or a segment thereof. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the marker proteins. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a marker protein which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the marker proteins from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of segments of a marker protein can be used to generate a variegated population of polypeptides for screening and subsequent selection of variant marker proteins or segments thereof. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

Another aspect of the invention pertains to antibodies directed against a protein of the invention. In preferred embodiments, the antibodies specifically bind a marker protein or a fragment thereof. The terms “antibody” and “antibodies” as used interchangeably herein refer to immunoglobulin molecules as well as fragments and derivatives thereof that comprise an immunologically active portion of an immunoglobulin molecule, (i.e., such a portion contains an antigen binding site which specifically binds an antigen, such as a marker protein, e.g., an epitope of a marker protein). An antibody which specifically binds to a protein of the invention is an antibody which binds the protein, but does not substantially bind other molecules in a sample, e.g., a biological sample, which naturally contains the protein. Examples of an immunologically active portion of an immunoglobulin molecule include, but are not limited to, single-chain antibodies (scAb), F(ab) and F(ab′)₂ fragments.

An isolated protein of the invention or a fragment thereof can be used as an immunogen to generate antibodies. The full-length protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the proteins of the invention, and encompasses at least one epitope of the protein such that an antibody raised against the peptide forms a specific immune complex with the protein. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. In preferred embodiments, an isolated marker protein or fragment thereof is used as an immunogen.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized protein or peptide. The preparation can further include an adjuvant, such as Freund's complete or incomplete adjuvant, or a similar immunostimulatory agent. Preferred immunogen compositions are those that contain no other human proteins such as, for example, immunogen compositions made using a non-human host cell for recombinant expression of a protein of the invention. In such a manner, the resulting antibody compositions have reduced or no binding of human proteins other than a protein of the invention.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. Preferred polyclonal and monoclonal antibody compositions are ones that have been selected for antibodies directed against a protein of the invention. Particularly preferred polyclonal and monoclonal antibody preparations are ones that contain only antibodies directed against a marker protein or fragment thereof.

Polyclonal antibodies can be prepared by immunizing a suitable subject with a protein of the invention as an immunogen The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a protein of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

The invention also provides recombinant antibodies that specifically bind a protein of the invention. In preferred embodiments, the recombinant antibodies specifically binds a marker protein or fragment thereof. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng. 7:1017-1026 and U.S. Pat. No. 6,121,424.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The antibodies of the invention can be isolated after production (e.g., from the blood or serum of the subject) or synthesis and further purified by well-known techniques. For example, IgG antibodies can be purified using protein A chromatography. Antibodies specific for a protein of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein of the invention.

In a preferred embodiment, the substantially purified antibodies of the invention may specifically bind to a signal peptide, a secreted sequence, an extracellular domain, a transmembrane or a cytoplasmic domain or cytoplasmic membrane of a protein of the invention. In a particularly preferred embodiment, the substantially purified antibodies of the invention specifically bind to a secreted sequence or an extracellular domain of the amino acid sequences of a protein of the invention. In a more preferred embodiment, the substantially purified antibodies of the invention specifically bind to a secreted sequence or an extracellular domain of the amino acid sequences of a marker protein.

An antibody directed against a protein of the invention can be used to isolate the protein by standard techniques, such as affinity chromatography or immunoprecipitation. Moreover, such an antibody can be used to detect the marker protein or fragment thereof (e.g., in a cellular lysate or cell supernatant) in order to evaluate the level and pattern of expression of the marker. The antibodies can also be used diagnostically to monitor protein levels in tissues or body fluids (e.g. in a pervasive developmental disorder-associated body fluid) as part of a clinical testing procedure, e.g., to, for example, determine the efficacy of a given treatment regimen. Detection can be facilitated by the use of an antibody derivative, which comprises an antibody of the invention coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

Antibodies of the invention may also be used as therapeutic agents in treating pervasive developmental disorders. In a preferred embodiment, completely human antibodies of the invention are used for therapeutic treatment of human patients suffering from a pervasive developmental disorder. In another preferred embodiment, antibodies that bind specifically to a marker protein or fragment thereof are used for therapeutic treatment. Further, such therapeutic antibody may be an antibody derivative or immunotoxin comprising an antibody conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugated antibodies of the invention can be used for modifying a given biological response, for the drug moiety is not to be construed as limited to classical chemical therapeutic agents. For example, the drug moiety may be a protein or polypeptide possessing a desired biological activity. Such proteins may include, for example, a toxin such as ribosome-inhibiting protein (see Better et al., U.S. Pat. No. 6,146,631, the disclosure of which is incorporated herein in its entirety), abrin, ricin A, pseudomonas exotoxin, or diphtheria toxin; a protein such as tumor necrosis factor, .alpha.-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Accordingly, in one aspect, the invention provides substantially purified antibodies, antibody fragments and derivatives, all of which specifically bind to a protein of the invention and preferably, a marker protein. In various embodiments, the substantially purified antibodies of the invention, or fragments or derivatives thereof, can be human, non-human, chimeric and/or humanized antibodies. In another aspect, the invention provides non-human antibodies, antibody fragments and derivatives, all of which specifically bind to a protein of the invention and preferably, a marker protein. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies. In still a further aspect, the invention provides monoclonal antibodies, antibody fragments and derivatives, all of which specifically bind to a protein of the invention and preferably, a marker protein. The monoclonal antibodies can be human, humanized, chimeric and/or non-human antibodies.

The invention also provides a kit containing an antibody of the invention conjugated to a detectable substance, and instructions for use. Still another aspect of the invention is a pharmaceutical composition comprising an antibody of the invention. In one embodiment, the pharmaceutical composition comprises an antibody of the invention and a pharmaceutically acceptable carrier.

### 3. Sequences of Markers of the Invention

Information about the markers of the invention are described in detail in below. Sequences of the markers of the invention are listed in the concurrently filed Sequence Listing.

### AHSA1

Official Symbol: AHSA1
Official Name: AHA1, activator of heat shock 90 kDa protein ATPase homolog 1 (yeast)
Gene ID: 10598
Organism: Homo sapiens
Other Aliases: HSPC322, AHA1, C14orf3, p38
Other Designations: activator of 90 kDa heat shock protein ATPase homolog 1
Nucleotide sequence:
   NCBI Reference Sequence: NM_012111.2
   LOCUS: NM_012111
   ACCESSION: NM_012111
   VERSION NM_012111.2 GI:224451069
   SEQ ID NO: 1
Protein sequence:
   NCBI Reference Sequence: NP_036243.1
   LOCUS NP_036243
   ACCESSION NP_036243
   VERSION NP_036243 GI:6912280
   SEQ ID NO: 2

### AHSG

Official Symbol: AHSG
Official Name: alpha-2-HS-glycoprotein
Gene ID: 197
Organism: Homo sapiens
Other Aliases: PRO2743, A2HS, AHS, FETUA, HSGA
Other Designations: alpha-2-Z-globulin; ba-alpha-2-glycoprotein; fetuin-A
Nucleotide sequence:
   NCBI Reference Sequence: NM_001622.2
   LOCUS: NM_001622
   ACCESSION: NM_001622
   VERSION NM_001622.2 GI:156523969
   SEQ ID NO: 3
Protein sequence:
   NCBI Reference Sequence: NP_001613.2
   LOCUS NP_001613
   ACCESSION NP_001613
   VERSION NP_001613.2 GI:156523970
   SEQ ID NO: 4

### ANXA6

Official Symbol: ANXA6
Official Name: annexin A6
Gene ID: 309
Organism: Homo sapiens
Other Aliases: ANX6, CBP68
Other Designations: 67 kDa calelectrin; CPB-II; annexin VI (p68); annexin-6; calcium-binding protein p68; calelectrin; calphobindin II; calphobindin-II; chromobindin-20; lipocortin VI; p68; p70
Nucleotide sequence: transcript variant 1
   NCBI Reference Sequence: NM_001155.4
   LOCUS: NM_001155
   ACCESSION: NM_001155
   VERSION NM_001155.4 GI:302129650
   SEQ ID NO: 5
Protein sequence: isoform 1
   NCBI Reference Sequence: NP_001146.2
   LOCUS NP_001146
   ACCESSION NP_001146
   VERSION NP_001146.2 GI:71773329
   SEQ ID NO: 6
Nucleotide sequence: transcript variant 2
   NCBI Reference Sequence: NM_001193544.1
   LOCUS: NM_001193544
   ACCESSION: NM_001193544
   VERSION NM_001193544.1 GI:302129651
   SEQ ID NO: 7
Protein sequence: isoform 2
   NCBI Reference Sequence: NP_001180473.1
   LOCUS NP_001180473
   ACCESSION NP_001180473
   VERSION NP_001180473.1 GI:302129652
   SEQ ID NO: 8

### AP1S1

Official Symbol: AP1S1
Official Name: adaptor-related protein complex 1, sigma 1 subunit
Gene ID: 1174
Organism: Homo sapiens
Other Aliases: AP19, CLAPS1, MEDNIK, SIGMA1A, WUGSC:H_DJ0747G18.2
Other Designations: AP-1 complex subunit sigma-1A; HA1 19 kDa subunit; adapter-related protein complex 1 sigma-1A subunit; clathrin assembly protein complex 1 sigma-1A small chain; clathrin coat assembly protein AP19; clathrin-associated/assembly/adaptor protein, small 1 (19 kD); golgi adaptor HA1/AP1 adaptin sigma-1A subunit; sigma1A subunit of AP-1 clathrin adaptor complex; sigma1A-adaptin
Nucleotide sequence:
   NCBI Reference Sequence: NM_001283.3
   LOCUS: NM_001283
   ACCESSION: NM_001283
   VERSION NM_001283.3 GI:148536831
   SEQ ID NO: 9
Protein sequence:
   NCBI Reference Sequence: NP_001274.1
   LOCUS NP_001274
   ACCESSION NP_001274
   VERSION NP_001274.1 GI:4557471
   SEQ ID NO: 10

### APMAP

Official Symbol: APMAP
Official Name: adipocyte plasma membrane associated protein
Gene ID: 57136
Organism: Homo sapiens
Other Aliases: RP4-568C11.2, BSCv, C20orf3
Other Designations: adipocyte plasma membrane-associated protein; protein BSCv
Nucleotide sequence:
   NCBI Reference Sequence: NM_020531.2
   LOCUS: NM_020531
   ACCESSION: NM_020531
   VERSION NM_020531.2 GI:41327713
   SEQ ID NO: 11
Protein sequence:
   NCBI Reference Sequence: NP_065392.1
   LOCUS NP_065392
   ACCESSION NP_065392
   VERSION NP_065392.1 GI:24308201
   SEQ ID NO: 12

### CAPG

Official Symbol: CAPG
Official Name: capping protein (actin filament), gelsolin-like
Gene ID: 822
Organism: Homo sapiens
Other Aliases: AFCP, MCP
Other Designations: actin regulatory protein CAP-G; actin-regulatory protein CAP-G; gelsolin-like capping protein; macrophage capping protein; macrophage-capping protein
Nucleotide sequence: transcript variant 2
   NCBI Reference Sequence: NM_001256139.1
   LOCUS: NM_001256139
   ACCESSION: NM_001256139
   VERSION NM_001256139.1 GI:371502124
   SEQ ID NO: 13
Protein sequence: isoform 1
   NCBI Reference Sequence: NP_001243068.1
   LOCUS NP_001243068
   ACCESSION NP_001243068
   VERSION NP_001243068.1 GI:371502125
   SEQ ID NO: 14
Nucleotide sequence: transcript variant 3
   NCBI Reference Sequence: NM_001256140.1
   LOCUS: NM_001256140
   ACCESSION: NM_001256140
   VERSION NM_001256140.1 GI:371502126
   SEQ ID NO: 15
Protein sequence: isoform 2
   NCBI Reference Sequence: NP_001243069.1
   LOCUS NP_001243069
   ACCESSION NP_001243069
   VERSION NP_001243069.1 GI:371502127
   SEQ ID NO: 16
Nucleotide sequence: transcript variant 1
   NCBI Reference Sequence: NM_001747.3
   LOCUS: NM_001747
   ACCESSION: NM_001747
   VERSION NM_001747 0.3 GI:371502123
   SEQ ID NO: 17
Protein sequence: isoform 1
   NCBI Reference Sequence: NP_001738.2
   LOCUS NP_001738
   ACCESSION NP_001738
   VERSION NP_001738.2 GI:63252913
   SEQ ID NO: 18

### CORO1A

Official Symbol: CORO1A
Official Name: coronin, actin binding protein, 1A
Gene ID: 11151
Organism: Homo sapiens
Other Aliases: CLABP, CLIPINA, HCORO1, TACO, p57
Other Designations: clipin-A; coronin-1; coronin-1A; coronin-like protein A; coronin-like protein p57; tryptophan aspartate-containing coat protein
Nucleotide sequence:
   NCBI Reference Sequence: NM_001193333.2
   LOCUS: NM_001193333
   ACCESSION: NM_001193333
   VERSION NM_001193333.2 GI:306482594
   SEQ ID NO: 19
Protein sequence:
   NCBI Reference Sequence: NP_001180262.1
   LOCUS NP_001180262
   ACCESSION NP_001180262
   VERSION NP_001180262.1 GI:300934762
   SEQ ID NO: 20
Nucleotide sequence: transcript variant 2
   NCBI Reference Sequence: NM_007074.3
   LOCUS: NM_007074
   ACCESSION: NM_007074
   VERSION NM_007074.3 GI:306482593
   SEQ ID NO: 21
Protein sequence:
   NCBI Reference Sequence: NP_009005.1
   LOCUS NP_009005
   ACCESSION NP_009005
   VERSION NP_009005.1 GI:5902134
   SEQ ID NO: 22

### COTL1

Official Symbol: COTL1
Official Name: coactosin-like 1 (Dictyostelium)
Gene ID: 23406
Organism: Homo sapiens
Other Aliases: CLP
Other Designations: coactosin-like protein
Nucleotide sequence:
   NCBI Reference Sequence: NM_021149.2
   LOCUS: NM_021149
   ACCESSION: NM_021149
   VERSION NM_021149.2 GI:23510452
   SEQ ID NO: 23
Protein sequence:
   NCBI Reference Sequence: NP_066972.1
   LOCUS NP_066972
   ACCESSION NP_066972
   VERSION NP_066972.1 GI:21624607
   SEQ ID NO: 24

### CPDX

Official Symbol: CPDX
Official Name:
Gene ID: 1371
Organism: Homo sapiens
Other Aliases: CPO, CPX, HCP
Other Designations: COX; coprogen oxidase; coproporphyrinogen-III oxidase, mitochondrial; coproporphyrinogenase
Nucleotide sequence:
   NCBI Reference Sequence: NM_000097.5
   LOCUS: NM_000097
   ACCESSION: NM_000097
   VERSION NM_000097.5 GI:261862333
   SEQ ID NO: 25
Protein sequence:
   NCBI Reference Sequence: NP_000088.3
   LOCUS NP_000088
   ACCESSION NP_000088
   VERSION NP_000088.3 GI:41393599
   SEQ ID NO: 26

### CPSF6

Official Symbol: CPSF6
Official Name: cleavage and polyadenylation specific factor 6, 68 kDa
Gene ID: 11052
Organism: Homo sapiens
Other Aliases: CFIM, CFIM68, HPBRII-4, HPBRII-7
Other Designations: CPSF 68 kDa subunit; cleavage and polyadenylation specificity factor 68 kDa subunit; cleavage and polyadenylation specificity factor subunit 6; pre-mRNA cleavage factor I, 68 kD subunit; pre-mRNA cleavage factor Im (68 kD); pre-mRNA cleavage factor Im 68 kDa subunit; protein HPBRII-4/7
Nucleotide sequence:
   NCBI Reference Sequence: NM_007007.2
   LOCUS: NM_007007
   ACCESSION: NM_007007
   VERSION NM_007007.2 GI:162329582
   SEQ ID NO: 27
Protein sequence:
   NCBI Reference Sequence: NP_008938.2
   LOCUS NP_008938
   ACCESSION NP_008938
   VERSION NP_008938.2 GI:162329583
   SEQ ID NO: 28

### CUX1

Official Symbol: CUX1
Official Name: cut-like homeobox 1
Gene ID: 1523
Organism: Homo sapiens
Other Aliases: CASP, CDP, CDP/Cut, CDP1, COY1, CUTL1, CUX, Clox, Cux/CDP, GOLIM6, Nbla10317, p100, p110, p200, p75
Other Designations: CCAAT displacement protein; cut homolog; golgi integral membrane protein 6; homeobox protein cux-1; protein CASP; putative protein product of Nbla10317
Nucleotide sequence: transcript variant 4
   NCBI Reference Sequence: NM_001202543.1
   LOCUS: NM_001202543
   ACCESSION: NM_001202543
   VERSION: NM_001202543.1 GI:321400106
   SEQ ID NO: 29
Protein sequence: isoform d
   NCBI Reference Sequence: NP_001189472.1
   LOCUS NP_001189472
   ACCESSION NP_001189472
   VERSION: NP_007189472.1 GI:321400107
   SEQ ID NO: 30
Nucleotide sequence: transcript variant 5
   NCBI Reference Sequence: NM_001202544.1
   LOCUS: NM_001202544
   ACCESSION: NM_001202544
   VERSION: NM_001202544.1 GI:321400111
   SEQ ID NO: 31
Protein sequence: isoform e
   NCBI Reference Sequence: NP_001189473.1
   LOCUS NP_001189473
   ACCESSION NP_001189473
   VERSION: NP_001189473.1 GI:321400112
   SEQ ID NO: 32
Nucleotide sequence: transcript variant 6
   NCBI Reference Sequence: NM_001202545.1
   LOCUS: NM_001202545
   ACCESSION: NM_001202545 XR_108855 XR_110720 XR_113043 XR_114073
   VERSION: NM_001202545.1 GI:321400113
   SEQ ID NO: 33
Protein sequence: isoform f
   NCBI Reference Sequence: NP_001189474.1
   LOCUS NP_001189474
   ACCESSION NP_001189474
   VERSION: NP_001189474.1 GI:321400114
   SEQ ID NO: 34
Nucleotide sequence: transcript variant 7
   NCBI Reference Sequence: NM_001202546.1
   LOCUS: NM_001202546
   ACCESSION: NM_001202546
   VERSION: NM_001202546.1 GI:321400115
   SEQ ID NO: 35
Protein sequence: isoform g
   NCBI Reference Sequence: NP_001189475.1
   LOCUS NP_001189475
   ACCESSION NP_001189475
   VERSION: NP_001189475.1 GI:321400116
   SEQ ID NO: 36
Nucleotide sequence: transcript variant 2
   NCBI Reference Sequence: NM_001913.3
   LOCUS: NM_001913
   ACCESSION: NM_001913
   VERSION: NM_001913.3 GI:321400109
   SEQ ID NO: 37
Protein sequence: isoform b
   NCBI Reference Sequence: NP_001904.2
   LOCUS NP_001904
   ACCESSION NP_001904
   VERSION: NP_001904.2 GI:31652236
   SEQ ID NO: 38
Nucleotide sequence: transcript variant 3
   NCBI Reference Sequence: NM_181500.2
   LOCUS: NM_181500
   ACCESSION: NM_181500
   VERSION: NM_181500.2 GI:321400110
   SEQ ID NO: 39
Protein sequence: isoform c
   NCBI Reference Sequence: NP_852477.1
   LOCUS NP_852477
   ACCESSION NP_852477
   VERSION: NP_852477.1 GI:31652238
   SEQ ID NO: 40
Nucleotide sequence: transcript variant 1
   NCBI Reference Sequence: NM_181552.3
   LOCUS: NM_181552
   ACCESSION: NM_181552
   VERSION: NM_181552.3 GI:321400108
   SEQ ID NO: 41
Protein sequence: isoform a
   NCBI Reference Sequence: NP_853530.2
   LOCUS NP_853530
   ACCESSION NP_853530
   VERSION: NP_853530.2 GI:148277064
   SEQ ID NO: 42

### DDX39A

Official Symbol: DDX39A
Official Name: DEAD (Asp-Glu-Ala-Asp) box polypeptide 39A (“DEAD” disclosed as SEQ ID NO: 244)
Gene ID: 10212
Organism: Homo sapiens
Other Aliases: BAT1, BAT1L, DDX39, DDXL, URH49
Other Designations: ATP-dependent RNA helicase DDX39A; DEAD (Asp-Glu-Ala-Asp) (SEQ ID NO: 244) box polypeptide 39 transcript; DEAD (SEQ ID NO: 244) box protein 39; DEAD/H (Asp-Glu-Ala-Asp/His) (SEQ ID NO: 245) box polypeptide 39; UAP56-related helicase, 49 kDa; nuclear RNA helicase URH49; nuclear RNA helicase, DECD variant (SEQ ID NO: 246) of DEAD box family (“DEAD” disclosed as SEQ ID NO: 244)
Nucleotide sequence:
   NCBI Reference Sequence: NM_005804.3
   LOCUS: NM_005804
   ACCESSION: NM_005804
   VERSION NM_005804.3 GI:308522777
   SEQ ID NO: 43
Protein sequence:
   NCBI Reference Sequence: NP_005795.2
   LOCUS NP_005795
   ACCESSION NP_005795
   VERSION NP_005795.2 GI:21040371
   SEQ ID NO: 44

### DDX6

Official Symbol: DDX6
Official Name: DEAD (Asp-Glu-Ala-Asp) box helicase 6 (“DEAD” disclosed as SEQ ID NO: 244)
Gene ID: 1656
Organism: Homo sapiens
Other Aliases: HLR2, P54, RCK
Other Designations: ATP-dependent RNA helicase p54; DEAD (Asp-Glu-Ala-Asp) (SEQ ID NO: 244) box polypeptide 6; DEAD (SEQ ID NO: 244) box protein 6; DEAD (SEQ ID NO: 244) box-6; DEAD/H (Asp-Glu-Ala-Asp/His) (SEQ ID NO: 245) box polypeptide 6 (RNA helicase, 54 kD); oncogene RCK; probable ATP-dependent RNA helicase DDX6
Nucleotide sequence: transcript variant 2
   NCBI Reference Sequence: NM_001257191.1
   LOCUS: NM_001257191
   ACCESSION: NM_001257191
   VERSION: NM_001257191.1 GI:380692341
   SEQ ID NO: 45
Protein sequence:
   NCBI Reference Sequence: NP_001244120.1
   LOCUS NP_001244120
   ACCESSION NP_001244120
   VERSION: NP_001244120.1 GI:380692342
   SEQ ID NO: 46
Nucleotide sequence: transcript variant 1
   NCBI Reference Sequence: NM_004397.4
   LOCUS: NM_004397
   ACCESSION: NM_004397
   VERSION: NM_004397.4 GI:164664517
   SEQ ID NO: 47
Protein sequence:
   NCBI Reference Sequence: NP_004388.2
   LOCUS NP_004388
   ACCESSION NP_004388
   VERSION: NP_004388.2 GI:164664518
   SEQ ID NO: 48

### DIABLO

Official Symbol: DIABLO
Official Name: diablo, IAP-binding mitochondrial protein
Gene ID: 56616
Organism: Homo sapiens
Other Aliases: hCG_1782202, DFNA64, DIABLO-S, SMAC, SMAC3
Other Designations: 0610041G12Rik; diablo homolog, mitochondrial; direct IAP-binding protein with low pI; mitochondrial Smac protein; second mitochondria-derived activator of caspase
Nucleotide sequence: mitochondrial isoform 1 precursor
   NCBI Reference Sequence: NM_019887.4
   LOCUS: NM_019887
   ACCESSION: NM_019887
   VERSION: NM_019887.4 GI:218505810
   SEQ ID NO: 49
Protein sequence: Isoform 1
   NCBI Reference Sequence: NP_063940.1
   LOCUS NP_063940
   ACCESSION: NP_063940
   VERSION: NP_063940.1 GI:9845297
   SEQ ID NO: 50
Nucleotide sequence: mitochondrial isoform 3 precursor
   NCBI Reference Sequence: NM_138929.3
   LOCUS: NM_138929
   ACCESSION: NM_138929
   VERSION: NM_138929.3 GI:218505811
   SEQ ID NO: 51
Protein sequence: Isoform 3
   NCBI Reference Sequence: NP_620307.1
   LOCUS: NP_620307
   ACCESSION: NP_620307
   VERSION: NP_620307.1 GI:21070976
   SEQ ID NO: 52

### EIF3B

Official Symbol: EIF3B
Official Name: eukaryotic translation initiation factor 3, subunit B
Gene ID: 8662
Organism: Homo sapiens
Other Aliases: EIF3-ETA, EIF3-P110, EIF3-P116, EIF3S9, PRT1
Other Designations: eIF-3-eta; eIF3 p110; eIF3 p116; eukaryotic translation initiation factor 3 subunit 9; eukaryotic translation initiation factor 3 subunit B; eukaryotic translation initiation factor 3, subunit 9 (eta, 116 kD); eukaryotic translation initiation factor 3, subunit 9 eta, 116 kDa; hPrt1; prt1 homolog
Nucleotide sequence:
   NCBI Reference Sequence: NM_001037283.1
   LOCUS: NM_001037283
   ACCESSION: NM_001037283
   VERSION: NM_001037283.1 GI:83367071
   SEQ ID NO: 53
Protein sequence:
   NCBI Reference Sequence: NP_001032360.1
   LOCUS NP_001032360
   ACCESSION NP_001032360
   VERSION: NP_001032360.1 GI:83367072
   SEQ ID NO: 54
Nucleotide sequence:
   NCBI Reference Sequence: NM_003751.3
   LOCUS: NM_003751
   ACCESSION: NM_003751
   VERSION: NM_003751.3 GI:83367073
   SEQ ID NO: 55
Protein sequence:
   NCBI Reference Sequence: NP_003742.2
   LOCUS NP_003742
   ACCESSION NP_003742
   VERSION: NP_003742.2 GI:33239445
   SEQ ID NO: 56

### EIF3G

Official Symbol: EIF3G
Official Name: eukaryotic translation initiation factor 3, subunit G
Gene ID: 8666
Organism: Homo sapiens
Other Aliases: EIF3-P42, EIF3S4, eIF3-delta, eIF3-p44
Other Designations: eIF-3 RNA-binding subunit; eIF-3-delta; eIF3 p42; eIF3 p44; eukaryotic translation initiation factor 3 RNA-binding subunit; eukaryotic translation initiation factor 3 subunit 4; eukaryotic translation initiation factor 3 subunit G; eukaryotic translation initiation factor 3 subunit p42; eukaryotic translation initiation factor 3, subunit 4 (delta, 44 kD); eukaryotic translation initiation factor 3, subunit 4 delta, 44 kDa
Nucleotide sequence:
   NCBI Reference Sequence: NM_003755.3
   LOCUS: NM_003755
   ACCESSION: NM_003755
   VERSION: NM_003755.3 GI:83281440
   SEQ ID NO: 57
Protein sequence:
   NCBI Reference Sequence: NP_003746.2
   LOCUS NP_003746
   ACCESSION NP_003746
   VERSION: NP_003746.2 GI:49472822
   SEQ ID NO: 58

### EIF3L

Official Symbol: EIF3L
Official Name: eukaryotic translation initiation factor 3, subunit L
Gene ID: 51386
Organism: Homo sapiens
Other Aliases: AL022311.1, EIF3EIP, EIF3S11, EIF3S6IP, HSPC021, HSPC025, MSTP005
Other Designations: eIEF associated protein HSPC021; eukaryotic translation initiation factor 3 subunit 6-interacting protein; eukaryotic translation initiation factor 3 subunit E-interacting protein; eukaryotic translation initiation factor 3 subunit L
Nucleotide sequence: Isoform 1
   NCBI Reference Sequence: NM_016091.3
   LOCUS: NM_016091
   ACCESSION: NM_016091
   VERSION: NM_016091.3 GI:339275829
   SEQ ID NO: 59
Protein sequence: Isoform 1
   NCBI Reference Sequence: NP_057175.1
   LOCUS NP_057175
   ACCESSION NP_057175
   VERSION: NP_0.57175.1 GI:7705433
   SEQ ID NO: 60
Nucleotide sequence: Isoform 2
   NCBI Reference Sequence: NM_001242923.1
   LOCUS: NM_001242923
   ACCESSION: NM_001242923
   VERSION: NM_001242923.1 GI:339275830
   SEQ ID NO: 61
Protein sequence: Isoform 2
   NCBI Reference Sequence: NP_001229852.1
   LOCUS NP_001229852
   ACCESSION NP_001229852
   VERSION: NP_001229852.1 GI:339275831
   SEQ ID NO: 62

### EIF4A2

Official Symbol: EIF4A2
Official Name: eukaryotic translation initiation factor 4A2
Gene ID: 1974
Organism: Homo sapiens
Other Aliases: BM-010, DDX2B, EIF4A, EIF4F, eIF-4A-II, eIF4A-II
Other Designations: ATP-dependent RNA helicase eIF4A-2; eukaryotic initiation factor 4A-II; eukaryotic translation initiation factor 4A
Nucleotide sequence:
   NCBI Reference Sequence: NM_001967.3
   LOCUS: NM_001967
   ACCESSION: NM_001967
   VERSION: NM_001967.3 GI:83700234
   SEQ ID NO: 63
Protein sequence:
   NCBI Reference Sequence: NP_001958.2
   LOCUS NP_001958
   ACCESSION NP_001958
   VERSION: NP_001958.2 GI:83700235
   SEQ ID NO: 64

### ERAP1

Official Symbol: ERAP1
Official Name: endoplasmic reticulum aminopeptidase 1
Gene ID: 51752
Organism: Homo sapiens
Other Aliases: UNQ584/PRO1154, A-LAP, ALAP, APPILS, ARTS-1, ARTS1, ERAAP, ERAAP1, PILS-AP, PILSAP
Other Designations: adipocyte-derived leucine aminopeptidase; aminopeptidase PILS; aminopeptidase regulator of TNFR1 shedding; endoplasmic reticulum aminopeptidase associated with antigen processing; puromycin-insensitive leucyl-specific aminopeptidase; type 1 tumor necrosis factor receptor shedding aminopeptidase regulator
Nucleotide sequence: Transcript variant 2
   NCBI Reference Sequence: NM_001040458.1
   LOCUS: NM_001040458
   ACCESSION: NM_001040458
   VERSION: NM_001040458.1 GI:94818890
   SEQ ID NO: 65
Protein sequence: Variant 2
   NCBI Reference Sequence: NP_001035548.1
   LOCUS NP_001035548
   ACCESSION NP_001035548
   VERSION: NP_001035548.1 GI:94818891
   SEQ ID NO: 66
Nucleotide sequence: Transcript variant 1
   NCBI Reference Sequence: NM_016442.3
   LOCUS: NM_016442
   ACCESSION: NM_016442
   VERSION: NM_016442.3 GI:94818900
   SEQ ID NO: 67
Protein sequence: Variant 1
   NCBI Reference Sequence: NP_057526.3
   LOCUS NP_057526
   ACCESSION NP_057526
   VERSION: NP_057526.3 GI:94818901
   SEQ ID NO: 68
Nucleotide sequence: Transcript variant 3
   NCBI Reference Sequence: NM_001198541.1
   LOCUS: NM_001198541
   ACCESSION: NM_001198541
   VERSION: NM_001198541.1 GI:309747090
   SEQ ID NO: 69
Protein sequence: Variant 3
   NCBI Reference Sequence: NP_001185470.1
   LOCUS NP_001185470
   ACCESSION NP_001185470
   VERSION: NP_001185470.1 GI:309747091
   SEQ ID NO: 70

### ERP44

Official Symbol: ERP44
Official Name: endoplasmic reticulum protein 44
Gene ID: 23071
Organism: Homo sapiens
Other Aliases: UNQ532/PRO1075, PDIA10, TXNDC4
Other Designations: ER protein 44; endoplasmic reticulum resident protein 44; endoplasmic reticulum resident protein 44 kDa; protein disulfide isomerase family A, member 10; thioredoxin domain containing 4 (endoplasmic reticulum); thioredoxin domain-containing protein 4
Nucleotide sequence:
   NCBI Reference Sequence: NM_015051.1
   LOCUS: NM_015051
   ACCESSION: NM_015051
   VERSION: NM_015051.1 GI:52487190
   SEQ ID NO: 71
Protein sequence:
   NCBI Reference Sequence: NP_055866.1
   LOCUS NP_055866
   ACCESSION NP_055866
   VERSION: NP_055866.1 GI:52487191
   SEQ ID NO: 72

### ETFB

Official Symbol: ETFB
Official Name: electron-transfer-flavoprotein, beta polypeptide
Gene ID: 2109
Organism: Homo sapiens
Other Aliases: FP585, MADD
Other Designations: beta-ETF; electron transfer flavoprotein beta subunit; electron transfer flavoprotein beta-subunit; electron transfer flavoprotein subunit beta; electron transfer flavoprotein, beta polypeptide; electron-transferring-flavoprotein, beta polypeptide
Nucleotide sequence: Isoform 1
   NCBI Reference Sequence: NM_001985.2
   LOCUS: NM_001985
   ACCESSION: NM_001985
   VERSION: NM_001985.2 GI:62420878
   SEQ ID NO: 73
Protein sequence: Isoform 1
   NCBI Reference Sequence: NP_001976.1
   LOCUS NP_001976
   ACCESSION NP_001976
   VERSION: NP_001976.1 GI:4503609
   SEQ ID NO: 74
Nucleotide sequence: Isoform 2
   NCBI Reference Sequence: NM_001014763.1
   LOCUS: NM_001014763
   ACCESSION: NM_001014763
   VERSION: NM_001014763.1 GI:62420876
   SEQ ID NO: 75
Protein sequence: Isoform 2
   NCBI Reference Sequence: NP_001014763.1
   LOCUS NP_001014763
   ACCESSION NP_001014763
   VERSION: NP_001014763.1 GI:62420877
   SEQ ID NO: 76

### FARSA

Official Symbol: FARSA
Official Name: phenylalanyl-tRNA synthetase, alpha subunit
Gene ID: 2193
Organism: Homo sapiens
Other Aliases: CML33, FARSL, FARSLA, FRSA, PheHA
Other Designations: pheRS; phenylalanine tRNA ligase 1, alpha, cytoplasmic; phenylalanine-tRNA ligase alpha chain; phenylalanine-tRNA ligase alpha subunit; phenylalanine-tRNA synthetase alpha-subunit; phenylalanine-tRNA synthetase-like, alpha subunit; phenylalanyl-tRNA synthetase alpha chain; phenylalanyl-tRNA synthetase-like, alpha subunit
Nucleotide sequence:
   NCBI Reference Sequence: NM_004461.2
   LOCUS: NM_004461
   ACCESSION: NM_004461
   VERSION: NM_004461.2 GI:126517492
   SEQ ID NO: 77
Protein sequence:
   NCBI Reference Sequence: NP_004452.1
   LOCUS NP_004452
   ACCESSION NP_004452
   VERSION: NP_004452.1 GI:4758340
   SEQ ID NO: 78

### FKBP4

Official Symbol: FKBP4
Official Name: FK506 binding protein 4, 59 kDa
Gene ID: 2288
Organism: Homo sapiens
Other Aliases: FKBP51, FKBP52, FKBP59, HBI, Hsp56, PPIase, p52
Other Designations: 51 kDa FK506-binding protein; FK506-binding protein 4 (59 kD); HSP binding immunophilin; T-cell FK506-binding protein, 59 kD; peptidylprolyl cis-trans isomerase FKBP4; peptidylprolyl cis-trans isomerase; rotamase
Nucleotide sequence:
   NCBI Reference Sequence: NM_002014.3
   LOCUS: NM_002014
   ACCESSION: NM_002014
   VERSION: NM_002014.3 GI:206725538
   SEQ ID NO: 79
Protein sequence:
   NCBI Reference Sequence: NP_002005.1
   LOCUS NP_002005
   ACCESSION NP_002005
   VERSION: NP_002005.1 GI:4503729
   SEQ ID NO: 80

### GET4

Official Symbol: GET4
Official Name: golgi to ER traffic protein 4 homolog
Gene ID: 51608
Organism: Homo sapiens
Other Aliases: CEE; TRC35; CGI-20; C7orf20
Other Designations: Golgi to ER traffic protein 4 homolog; H_NH1244M04.5; conserved edge expressed protein; conserved edge protein; conserved edge-expressed protein; transmembrane domain recognition complex 35 kDa subunit; transmembrane domain recognition complex, 35 kDa
Nucleotide sequence:
   NCBI Reference Sequence: NM_015949.2
   LOCUS: NM_015949
   ACCESSION: NM_015949
   VERSION: NM_015949.2 GI:38570061
   SEQ ID NO: 81
Protein sequence:
   NCBI Reference Sequence: NP_057033.2
   LOCUS: NP_057033
   ACCESSION: NP_057033
   VERSION: NP_057033.2 GI:38570062
   SEQ ID NO: 82

### GLUD1

Official Symbol: GLUD1
Official Name: glutamate dehydrogenase 1
Gene ID: 2746
Organism: Homo sapiens
Other Aliases: GDH; GDH1; GLUD
Other Designations: GDH 1; glutamate dehydrogenase (NAD(P)+); glutamate dehydrogenase 1, mitochondrial
Nucleotide sequence:
   NCBI Reference Sequence: NM_005271.3
   LOCUS: NM_005271
   ACCESSION: NM_005271
   VERSION: NM_005271.3 GI:260064010
   SEQ ID NO: 83
Protein sequence:
   NCBI Reference Sequence: NP_005262.1
   LOCUS: NP_005262
   ACCESSION: NP_005262
   VERSION: NP_005262.1 GI:4885281
   SEQ ID NO: 84

### GTF2I

Official Symbol: GTF2I
Official Name: general transcription factor IIi
Gene ID: 2969
Organism: Homo sapiens
Other Aliases: BAP135, BTKAP1, DIWS, GTFII-I, IB291, SPIN, TFII-I, WBS, WBSCR6
Other Designations: BTK-associated protein 135; BTK-associated protein, 135 kD; Bruton tyrosine kinase-associated protein 135; SRF-Phox1-interacting protein; Williams-Beuren syndrome chromosome region 6; general transcription factor II-I; williams-Beuren syndrome chromosomal region 6 protein
Nucleotide sequence: transcript variant 5
   NCBI Reference Sequence: NM_001163636.1
   LOCUS: NM_001163636
   ACCESSION: NM_001163636
   VERSION: NM_001163636.1 GI:254692933
   SEQ ID NO: 85
Protein sequence: isoform 5
   NCBI Reference Sequence: NP_001157108.1
   LOCUS: NP_001157108
   ACCESSION: NP_001157108
   VERSION: NP_001157108.1 GI:254692934
   SEQ ID NO: 86
Nucleotide sequence: transcript variant 4
   NCBI Reference Sequence: NM_001518.3
   LOCUS: NM_001518
   ACCESSION: NM_001518
   VERSION: NM_001518.3 GI:169881251
   SEQ ID NO: 87
Protein sequence: isoform 4
   NCBI Reference Sequence: NP_001509.3
   LOCUS: NP_001509
   ACCESSION: NP_001509 NP_127496 XP_944599
   VERSION: NP_001509.3 GI:169881252
   SEQ ID NO: 88
Nucleotide sequence: transcript variant 1
   NCBI Reference Sequence: NM_032999.2
   LOCUS: NM_032999
   ACCESSION: NM_032999
   VERSION: NM_032999.2 GI:169881253
   SEQ ID NO: 89
Protein sequence: isoform 1
   NCBI Reference Sequence: NP_127492.1
   LOCUS: NP_127492
   ACCESSION: NP_127492
   VERSION: NP_127492.1 GI:14670350
   SEQ ID NO: 90
Nucleotide sequence: transcript variant 2
   NCBI Reference Sequence: NM_033000.2
   LOCUS: NM_033000
   ACCESSION: NM_033000 XM_001133646
   VERSION: NM_033000.2 GI:169881254
   SEQ ID NO: 91
Protein sequence: isoform 2
   NCBI Reference Sequence: NP_127493.1
   LOCUS: NP_127493
   ACCESSION: NP_127493 XP_001133646
   VERSION: NP_127493.1 GI:14670352
   SEQ ID NO: 92
Nucleotide sequence: transcript variant 3
   NCBI Reference Sequence: NM_033001.2
   LOCUS: NM_033001
   ACCESSION: NM_033001 XM_001130609
   VERSION: NM_033001.2 GI:169881255
   SEQ ID NO: 93
Protein sequence: isoform 3
   NCBI Reference Sequence: NP_127494.1
   LOCUS: NP_127494
   ACCESSION: NP_127494 XP_001130609
   VERSION: NP_127494.1 GI:14670354
   SEQ ID NO: 94

### HBA2

Official Symbol: HBA2
Official Name: hemoglobin, alpha 2
Gene ID: 3040
Organism: Homo sapiens
Other Aliases: HBH
Other Designations: alpha globin; alpha-2 globin; alpha-globin; hemoglobin alpha chain; hemoglobin subunit alpha
Nucleotide sequence:
   NCBI Reference Sequence: NM_000517.4
   LOCUS: NM_000517
   ACCESSION: NM_000517
   VERSION: NM_000517.4 GI:172072689
   SEQ ID NO: 95
Protein sequence:
   NCBI Reference Sequence: NP_000508.1
   LOCUS: NP_000508
   ACCESSION: NP_000508
   VERSION: NP_000508.1 GI:4504345
   SEQ ID NO: 96

### HLA-A

Official Symbol: HLA-A
Official Name: major histocompatibility complex, class I, A
Gene ID: 3105
Organism: Homo sapiens
Other Aliases: DAQB-90C11.16-002, HLAA
Other Designations: HLA class I histocompatibility antigen, A-1 alpha chain; MHC class I antigen HLA-A heavy chain; antigen presenting molecule; leukocyte antigen class I-A
Nucleotide sequence: transcript variant 2
   NCBI Reference Sequence: NM_001242758.1
   LOCUS: NM_001242758
   ACCESSION: NM_0012427.58 XM_003960035 XM_003960036 XM_003960037 XM_003960038 XM_003960039 XM_003960040 XM_003960041 XM_003960042 XM_003960043 XM_003960044 XM_003960045
   VERSION: NM_001242758.1 GI:337752169
   SEQ ID NO: 97
Protein sequence: A*01:01:01:01 allele
   NCBI Reference Sequence: NP_001229687.1
   LOCUS: NP_001229687
   ACCESSION: NP_001229687 XP_003960084 XP_003960085 XP_003960086 XP_003960087 XP_003960088 XP_003960089 XP_003960090 XP_003960091 XP_003960092 XP_003960093 XP_003960094
   VERSION: NP_001229687.1 GI:337752170
   SEQ ID NO: 98
Nucleotide sequence: Transcript variant 1
   NCBI Reference Sequence: NM_002116.7
   LOCUS: NM_002116
   ACCESSION: NM_002116 NM_001080840 XM_001713645
   VERSION: NM_002116.7 GI:337752171
   SEQ ID NO: 99
Protein sequence: A*03:01:0:01 allele
   NCBI Reference Sequence: NP_002107.3
   LOCUS: NP_002107 NP_001074309 XP_001713697
   ACCESSION: NP_002107
   VERSION: NP_002107.3 GI:24797067
   SEQ ID NO: 100

### HLA-D0131

Official Symbol: HLA-DQB1
Official Name: major histocompatibility complex, class II, DQ beta
Gene ID: 3119
Organism: Homo sapiens
Other Aliases: DADB-249P12.2, CELIAC1, HLA-DQB, IDDM1
Other Designations: HLA class II histocompatibility antigen, DQ beta 1 chain; MHC DQ beta; MHC class II DQ beta chain; MHC class II HLA-DQ beta glycoprotein; MHC class II antigen DQB1; MHC class II antigen HLA-DQ-beta-1; MHC class2 antigen; lymphocyte antigen
Nucleotide sequence: transcript variant 2
   NCBI Reference Sequence: NM_001243961.1
   LOCUS: NM_001243961
   ACCESSION: NM_001243961
   VERSION: NM_001243961.1 GI:345461080
   SEQ ID NO: 101
Protein sequence: isoform 2
   NCBI Reference Sequence: NP_001230890.1
   LOCUS: NP_001230890
   ACCESSION: NP_001230890
   VERSION: NP_001230890.1 GI:345461081
   SEQ ID NO: 102
Nucleotide sequence: transcript variant 3
   NCBI Reference Sequence: NM_001243962.1
   LOCUS: NM_001243962
   ACCESSION: NM_001243962 XM_003846474 XM_003846475
   VERSION: NM_001243962.1 GI:345461078
   SEQ ID NO: 103
Protein sequence: isoform 1
   NCBI Reference Sequence: NP_001230891.1
   LOCUS: NP_001230891
   ACCESSION: NP_001230891 XP_003846522 XP_003846523
   VERSION: NP_001230891.1 GI:345461079
   SEQ ID NO: 104
Nucleotide sequence: transcript variant 1
   NCBI Reference Sequence: NM_002123.4
   LOCUS: NM_002123
   ACCESSION: NM_002123 XM_001722253 XM_001723447
   VERSION: NM_002123.4 GI:345461082
   SEQ ID NO: 105
Protein sequence: isoform 1
   NCBI Reference Sequence: NP_002114.3
   LOCUS: NP_002114
   ACCESSION: NP_002114 XP_001722305 XP_001723499
   VERSION: NP_002114.3 GI:150418002
   SEQ ID NO: 106

### HLA-DRA

Official Symbol: HLA-DRA
Official Name: major histocompatibility complex, class II, DR alpha
Gene ID: 3122
Organism: Homo sapiens
Other Aliases: DASS-397D15.1, HLA-DRA1, MLRW
Other Designations: HLA class II histocompatibility antigen, DR alpha chain; MHC cell surface glycoprotein; MHC class II antigen DRA; histocompatibility antigen HLA-DR alpha
Nucleotide sequence:
   NCBI Reference Sequence: NM_019111.4
   LOCUS: NM_019111
   ACCESSION: NM_019111
   VERSION: NM_019111.4 GI:301171411
   SEQ ID NO: 107
Protein sequence:
   NCBI Reference Sequence: NP_061984.2
   LOCUS: NP_061984
   ACCESSION: NP_061984
   VERSION: NP_061984.2 GI:52426774
   SEQ ID NO: 108

### HNRNPM

Official Symbol: HNRNPM
Official Name: heterogeneous nuclear ribonucleoprotein M
Gene ID: 4670
Organism: Homo sapiens
Other Aliases: CEAR, HNRNPM4, HNRPM, HNRPM4, HTGR1, NAGR1, hnRNP M
Other Designations: CEA receptor; N-acetylglucosamine receptor 1; heterogenous nuclear ribonucleoprotein M4; hnRNA-binding protein M4
Nucleotide sequence: transcript variant 1
   NCBI Reference Sequence: NM_005968.4
   LOCUS: NM_005968
   ACCESSION: NM_005968
   VERSION: NM_00.5968.4 GI:345091004
   SEQ ID NO: 109
Protein sequence: isoform a
   NCBI Reference Sequence: NP_005959.2
   LOCUS: NP_005959
   ACCESSION: NP_005959
   VERSION: NP_005959.2 GI:14141152
   SEQ ID NO: 110
Nucleotide sequence: transcript variant 2
   NCBI Reference Sequence: NM_031203.3
   LOCUS: NM_031203
   ACCESSION: NM_031203
   VERSION: NM_031203.3 GI:345091007
   SEQ ID NO: 111
Protein sequence: isoform b
   NCBI Reference Sequence: NP_112480.2
   LOCUS: NP_112480
   ACCESSION: NP_112480
   VERSION: NP_112480.2 GI:157412270
   SEQ ID NO: 112

### HPRT1

Official Symbol: HPRT1
Official Name: hypoxanthine phosphoribosyltransferase 1
Gene ID: 3251
Organism: Homo sapiens
Other Aliases: HGPRT, HPRT
Other Designations: HGPRTase; hypoxanthine-guanine phosphoribosyltransferase
Nucleotide sequence:
   NCBI Reference Sequence: NM_000194.2
   LOCUS: NM_000194
   ACCESSION: NM_000194
   VERSION: NM_000194.2 GI:164518913
   SEQ ID NO: 113
Protein sequence:
   NCBI Reference Sequence: NP_000185.1
   LOCUS: NP_000185
   ACCESSION: NP_000185
   VERSION: NP_000185.1 GI:4504483
   SEQ ID NO: 114

### HSP90B1

Official Symbol: HSP90B1
Official Name: heat shock protein 90 kDa beta (Grp94), member 1
Gene ID: 7184
Organism: Homo sapiens
Other Aliases: ECGP, GP96, GRP94, TRA1
Other Designations: 94 kDa glucose-regulated protein; endoplasmin; endothelial cell (HBMEC) glycoprotein; heat shock protein 90 kDa beta member 1; stress-inducible tumor rejection antigen gp96; tumor rejection antigen (gp96) 1; tumor rejection antigen 1
Nucleotide sequence:
   NCBI Reference Sequence: NM_003299.2
   LOCUS: NM_003299
   ACCESSION: NM_003299
   VERSION: NM_003299.2 GI:399567818
   SEQ ID NO: 115
Protein sequence:
   NCBI Reference Sequence: NP_003290.1
   LOCUS: NP_003290
   ACCESSION: NP_003290
   VERSION: NP_003290.1 GI:4507677
   SEQ ID NO: 116

### HSPH1

Official Symbol: HSPH1
Official Name: heat shock 105 kDa/110 kDa protein 1
Gene ID: 10808
Organism: Homo sapiens
Other Aliases: RP11-173P16.1, HSP105, HSP105A, HSP105B, NY-CO-25
Other Designations: antigen NY-CO-25; heat shock 105 kD alpha; heat shock 105 kD beta; heat shock 105 kDa protein 1; heat shock 110 kDa protein; heat shock protein 105 kDa
Nucleotide sequence:
   NCBI Reference Sequence: NM_006644.2
   LOCUS: NM_006644
   ACCESSION: NM_006644
   VERSION: NM_006644.2 GI:42544158
   SEQ ID NO: 117
Protein sequence:
   NCBI Reference Sequence: NP_006635.2
   LOCUS: NP_006635
   ACCESSION: NP_006635
   VERSION: NP_006635.2 GI:42544159
   SEQ ID NO: 118

### IGHM

Official Symbol: IGHM
Official Name: immunoglobulin heavy constant mu
Gene ID: 3507
Organism: Homo sapiens
Other Aliases: AGM1, MU, VH
Other Designations: none
Nucleotide sequence: mRNA variant 1
   ENA Sequence Reference No: X17115.1
   >ENA|X17115|X17115.1 Human mRNA for IgM heavy chain complete sequence: Location:1 . . . 1000
   SEQ ID NO: 119
Protein sequence: isoform 1
   UniProtKB/Swiss-Prot Reference No.: P01871-1
   >sp|P01871|IGHM_HUMAN Ig mu chain C region OS=Homo sapiens GN=IGHM PE=1 SV=3
   SEQ ID NO: 120
Nucleotide sequence: mRNA variant 2
   ENA Sequence Reference No: X57086.1
   >ENA|X57086|X57086.1 H. sapiens mRNA for IgM heavy chain constant domain: Location:1 . . . 1000
   SEQ ID NO: 121
Protein sequence: isoform 2
   UniProtKB/Swiss-Prot Reference No.: P01871-2
   >sp|P01871-2|IGHM_HUMAN Isoform 2 of Ig mu chain C region OS=Homo sapiens: GN=IGHM
   SEQ ID NO: 122

### IGLC1

Official Symbol: IGLC1
Official Name: immunoglobulin lambda constant 1 (Mcg marker)
Gene ID: 3537
Organism: Homo sapiens
Other Aliases: IGLC
Other Designations: none
Nucleotide sequence: mRNA variant 1
   ENA Sequence Reference No: CAA36047.1
      >ENA|CAA36047|CAA36047.1 Homo sapiens (human) hypothetical protein: Location:1 . . . 320
      SEQ ID NO: 123
Nucleotide sequence: mRNA variant 2
   ENA Sequence Reference No: AAA59106.1
   >ENA|AAA59106|AAA59106.1 Homo sapiens (human) partial immunoglobulin lambda light chain C region: Location: 1 . . . 315
   SEQ ID NO: 124
Protein sequence:
   UniProtKB/Swiss-Prot Reference No.: P0CG04
   >sp|P0CG04|LAC1_HUMAN Ig lambda-1 chain C regions OS=Homo sapiens GN=IGLC1 PE=1 SV=1
   SEQ ID NO: 125

### ITGB7

Official Symbol: ITGB7
Official Name: integrin, beta 7
Gene ID: 3695
Organism: Homo sapiens
Other Aliases: none
Other Designations: gut homing receptor beta subunit; integrin beta 7 subunit; integrin beta-7
Nucleotide sequence:
   NCBI Reference Sequence: NM_000889.1
   LOCUS: NM_000889
   ACCESSION: NM_000889
   VERSION: NM_000889.1 GI:4504776
   SEQ ID NO: 126
Protein sequence:
   NCBI Reference Sequence: NP_000880.1
   LOCUS: NP_000880
   ACCESSION: NP_000880
   VERSION: NP_000880.1 GI:4504777
   SEQ ID NO: 127

### LCP1

Official Symbol: LCP1
Official Name: lymphocyte cytosolic protein 1 (L-plastin)
Gene ID: 3936
Organism: Homo sapiens
Other Aliases: RP11-139H14.1, CP64, L-PLASTIN, LC64P, LPL, PLS2
Other Designations: L-plastin (Lymphocyte cytosolic protein 1) (LCP-1) (LC64P); LCP-1; Lymphocyte cytosolic protein-1 (plasmin); bA139H14.1 (lymphocyte cytosolic protein 1 (L-plastin)); plastin 2; plastin-2
Nucleotide sequence:
   NCBI Reference Sequence: NM_002298.4
   LOCUS: NM_002298
   ACCESSION: NM_002298
   VERSION: NM_002298.4 GI:195546923
   SEQ ID NO: 128
Protein sequence:
   NCBI Reference Sequence: NP_002289.2
   LOCUS: NP_002289
   ACCESSION: NP_002289
   VERSION: NP_002289.2 GI:167614506
   SEQ ID NO: 129

### LETM1

Official Symbol: LETM1
Official Name: leucine zipper-EF-hand containing transmembrane protein 1
Gene ID: 3954
Organism: Homo sapiens
Other Aliases: none
Other Designations: LETM1 and EF-hand domain-containing protein 1, mitochondrial; Mdm38 homolog; leucine zipper-EF-hand-containing transmembrane protein 1
Nucleotide sequence:
   NCBI Reference Sequence: NM_012318.2
   LOCUS: NM_012318
   ACCESSION: NM_012318
   VERSION: NM_012318.2 GI:194595498
   SEQ ID NO: 130
Protein sequence:
   NCBI Reference Sequence: NP_036450.1
   LOCUS: NP_036450
   ACCESSION: NP_036450
   VERSION: NP_036450.1 GI:6912482
   SEQ ID NO: 131

### LMNA

Official Symbol: LMNA
Official Name: 1amin A/C
Gene ID: 150330
Organism: Homo sapiens
Other Aliases: RP11-54H19.1, CDCD1, CDDC, CMD1A, CMT2B1, EMD2, FPL, FPLD, FPLD2, HGPS, IDC, LDP1, LFP, LGMD1B, LMN1, LMNC, LMNL1, PRO1
Other Designations: 70 kDa 1amin; 1amin; 1amin A/C-like 1; pre1amin-A/C; renal carcinoma antigen NY-REN-32
Nucleotide sequence: transcript variant 4
   NCBI Reference Sequence: NM_001257374.1
   LOCUS: NM_001257374
   ACCESSION: NM_001257374
   VERSION: NM_001257374.1 GI:383792149
   SEQ ID NO: 132
Protein sequence: isoform D
   NCBI Reference Sequence: NP_001244303.1
   LOCUS: NP_001244303
   ACCESSION: NP_001244303
   VERSION: NP_001244303.1 GI:383792150
   SEQ ID NO: 133
Nucleotide sequence: transcript variant 2
   NCBI Reference Sequence: NM_005572.3
   LOCUS: NM_005572
   ACCESSION: NM_005572
   VERSION: NM_005572.3 GI:153281091
   SEQ ID NO: 134
Protein sequence: isoform C
   NCBI Reference Sequence: NP_005563.1
   LOCUS: NP_005563
   ACCESSION: NP_005563
   VERSION: NP_005563.1 GI:5031875
   SEQ ID NO: 135
Nucleotide sequence: transcript variant 1
   NCBI Reference Sequence: NM_170707.3
   LOCUS: M_170707
   ACCESSION: NM_170707
   VERSION: NM_170707.3 GI:383792147
   SEQ ID NO: 136
Protein sequence: isoform A
   NCBI Reference Sequence: NP_733821.1
   LOCUS: NP_733821
   ACCESSION: NP_733821
   VERSION: NP_733821.1 GI:27436946
   SEQ ID NO: 137
Nucleotide sequence: transcript variant 3
   NCBI Reference Sequence: NM_170708.3
   LOCUS: NM_170708
   ACCESSION: NM_170708
   VERSION: NM_170708.3 GI:383792148
   SEQ ID NO: 138
Protein sequence: isoform A-delta10
   NCBI Reference Sequence: NP_733822.1
   LOCUS: NP_733822
   ACCESSION: NP_733822
   VERSION: NP_733822.1 GI:27436948
   SEQ ID NO: 139

### MGEA5

Official Symbol: MGEA5
Official Name: meningioma expressed antigen 5 (hyaluronidase)
Gene ID: 10724
Organism: Homo sapiens
Other Aliases: MEA5, NCOAT, OGA
Other Designations: O-GlcNAcase; bifunctional protein NCOAT; hyaluronidase in meningioma; meningioma-expressed antigen 5; nuclear cytoplasmic O-GlcNAcase and acetyltransferase
Nucleotide sequence: transcript variant 2
   NCBI Reference Sequence: NM_001142434.1
   LOCUS: NM_001142434
   ACCESSION: NM_001142434
   VERSION: NM_001142434.1 GI:215490055
   SEQ ID NO: 140
Protein sequence: isoform b
   NCBI Reference Sequence: NP_001135906.1
   LOCUS: NP_001135906
   ACCESSION: NP_00113.5906
   VERSION: NP_001135906.1 GI:215490056
   SEQ ID NO: 141
Nucleotide sequence: transcript variant 1
   NCBI Reference Sequence: NM_012215.3
   LOCUS: NM_012215
   ACCESSION: NM_012215
   VERSION: NM_012215.3 GI:215490054
   SEQ ID NO: 142
Protein sequence: isoform a
   NCBI Reference Sequence: NP_036347.1
   LOCUS: NP_036347
   ACCESSION: NP_036347
   VERSION: NP_036347.1 GI:11024698
   SEQ ID NO: 143

### MTHFD1

Official Symbol: MTHFD1
Official Name: methylenetetrahydrofolate dehydrogenase (NADP+ dependent) 1, methenyltetrahydrofolate cyclohydrolase, formyltetrahydrofolate synthetase
Gene ID: 4522
Organism: Homo sapiens
Other Aliases: MTHFC, MTHFD
Other Designations: 5,10-methylenetetrahydrofolate dehydrogenase, 5,10-methylenetetrahydrofolate cyclohydrolase, 10-formyltetrahydrofolate synthetase; C-1-tetrahydrofolate synthase, cytoplasmic; C1-THF synthase; cytoplasmic C-1-tetrahydrofolate synthase
Nucleotide sequence:
   NCBI Reference Sequence: NM_005956.3
   LOCUS: NM_005956
   ACCESSION: NM_005956
   VERSION: NM_005956.3 GI:222136638
   SEQ ID NO: 144
Protein sequence:
   NCBI Reference Sequence: NP_005947.3
   LOCUS: NP_005947
   ACCESSION: NP_005947
   VERSION: NP_005947.3 GI:222136639
   SEQ ID NO: 145

### MX1

Official Symbol: MX1
Official Name: myxovirus (influenza virus) resistance 1, interferon-inducible protein p78 (mouse)
Gene ID: 4599
Organism: Homo sapiens
Other Aliases: IFI-78K, IFI78, MX, MxA
Other Designations: interferon-induced GTP-binding protein Mx1; interferon-regulated resistance GTP-binding protein MxA; myxoma resistance protein 1
Nucleotide sequence: transcript variant 1
   NCBI Reference Sequence: NM_001144925.1
   LOCUS: NM_001144925
   ACCESSION: NM_001144925
   VERSION: NM_001144925.1 GI:222136618
   SEQ ID NO: 146
Protein sequence: all variants encode the same protein
   NCBI Reference Sequence: NP_001138397.1
   LOCUS: NP_001138397
   ACCESSION: NP_001138397
   VERSION: NP_001138397.1 GI:222136619
   SEQ ID NO: 147
Nucleotide sequence: transcript variant 3
   NCBI Reference Sequence: NM_001178046.1
   LOCUS: NM_001178046
   ACCESSION: NM_001178046
   VERSION: NM_001178046.1 GI:295842577
   SEQ ID NO: 148 protein sequence: all variants encode the same protein
   NCBI Reference Sequence: NP_001171517.1
   LOCUS: NP_001171517
   ACCESSION: NP_001171517
   VERSION: NP_001171517.1 GI:295842578
   SEQ ID NO: 149
Nucleotide sequence: transcript variant 2
   NCBI Reference Sequence: NM_002462.3
   LOCUS: NM_002462
   ACCESSION: NM_002462
   VERSION: NM_002462.3 GI:222136616
   SEQ ID NO: 150
Protein sequence: all variants encode the same protein
   NCBI Reference Sequence: NP_002453.2
   LOCUS: NP_002453
   ACCESSION: NP_002453
   VERSION: NP_002453.2 GI:222136617
   SEQ ID NO: 151

### OSBP

Official Symbol: OSBP
Official Name: oxysterol binding protein
Gene ID: 5007
Organism: Homo sapiens
Other Aliases: OSBP1
Other Designations: oxysterol-binding protein 1
Nucleotide sequence:
   NCBI Reference Sequence: NM_002556.2
   LOCUS: NM_002556
   ACCESSION: NM_002556
   VERSION: NM_002556.2 GI:34485728
   SEQ ID NO: 152
Protein sequence:
   NCBI Reference Sequence: NP_002547.1
   LOCUS: NP_002547
   ACCESSION: NP_002547
   VERSION: NP_002547.1 GI:4505531
   SEQ ID NO: 153

### P4HB

Official Symbol: P4HB
Official Name: prolyl 4-hydroxylase, beta polypeptide
Gene ID: 5034
Organism: Homo sapiens
Other Aliases: DSI, ERBA2L, GIT, P4Hbeta, PDI, PDIA1, PHDB, PO4DB, PO4HB, PROHB
Other Designations: cellular thyroid hormone-binding protein; collagen prolyl 4-hydroxylase beta; glutathione-insulin transhydrogenase; p55; procollagen-proline, 2-oxoglutarate 4-dioxygenase (proline 4-hydroxylase), beta polypeptide; prolyl 4-hydroxylase subunit beta; protein disulfide isomerase family A, member 1; protein disulfide isomerase-associated 1; protein disulfide isomerase/oxidoreductase; protein disulfide-isomerase; protocollagen hydroxylase; thyroid hormone-binding protein p55
Nucleotide sequence:
   NCBI Reference Sequence: NM_000918.3
   LOCUS: NM_000918
   ACCESSION: NM_000918
   VERSION: NM_000918.3 GI:121256637
   SEQ ID NO: 154
Protein sequence:
   NCBI Reference Sequence: NP_000909.2
   LOCUS: NP_000909
   ACCESSION: NP_000909
   VERSION: NP_000909.2 GI:20070125
   SEQ ID NO: 155

### PCNA

Official Symbol: PCNA
Official Name: proliferating cell nuclear antigen
Gene ID: 5111
Organism: Homo sapiens
Other Aliases: none
Other Designations: DNA polymerase delta auxiliary protein; cyclin
Nucleotide sequence: transcript variant 1
   NCBI Reference Sequence: NM_002592.2
   LOCUS: NM_002592
   ACCESSION: NM_002592
   VERSION: NM_002592.2 GI:33239449
   SEQ ID NO: 156
Protein sequence: both variants encode the same protein
   NCBI Reference Sequence: NP_002583.1
   LOCUS: NP_002583
   ACCESSION: NP_002583
   VERSION: NP_002583.1 GI:4505641
   SEQ ID NO: 157
Nucleotide sequence: transcript variant 2
   NCBI Reference Sequence: NM_{—} 182649.1
   LOCUS: NM_182649
   ACCESSION: NM_182649
   VERSION: NM_182649.1 GI:33239450
   SEQ ID NO: 158
Protein sequence: both variants encode the same protein
   NCBI Reference Sequence: NP_872590.1
   LOCUS: NP_872590
   ACCESSION: NP_872590
   VERSION: NP_872590.1 GI:33239451
   SEQ ID NO: 159

### PDCL3

Official Symbol: PDCL3
Official Name: phosducin-like 3
Gene ID: 79031
Organism: Homo sapiens
Other Aliases: HTPHLP, PHLP2A, PHLP3, VIAF, VIAF1
Other Designations: IAP-associated factor VIAF1; VIAF-1; phPL3; phosducin-like protein 3; viral IAP-associated factor 1
Nucleotide sequence:
   NCBI Reference Sequence: NM_024065.4
   LOCUS: NM_024065
   ACCESSION: NM_024065
   VERSION: NM_024065.4 GI:163310761
   SEQ ID NO: 160
Protein sequence:
   NCBI Reference Sequence: NP_076970.1
   LOCUS NP_076970
   ACCESSION NP_076970
   VERSION: NP_076970.1 GI:13129044
   SEQ ID NO: 161

### PDIA4

Official Symbol: PDIA4
Official Name: protein disulfide isomerase family A, member 4
Gene ID: 9601
Organism: Homo sapiens
Other Aliases: ERP70, ERP72, ERp-72
Other Designations: ER protein 70; ER protein 72; endoplasmic reticulum resident protein 70; endoplasmic reticulum resident protein 72; protein disulfide isomerase related protein (calcium-binding protein, intestinal-related); protein disulfide isomerase-associated 4; protein disulfide-isomerase A4
Nucleotide sequence:
   NCBI Reference Sequence: NM_004911.4
   LOCUS: NM_004911
   ACCESSION: NM_004911
   VERSION: NM_004911.4 GI:157427676
   SEQ ID NO: 162
Protein sequence:
   NCBI Reference Sequence: NP_004902.1
   LOCUS NP_004902
   ACCESSION NP_004902
   VERSION: NP_004902.1 GI:4758304
   SEQ ID NO: 163

### PEA15

Official Symbol: EA15
Official Name: phosphoprotein enriched in astrocytes 15
Gene ID: 8682
Organism: Homo sapiens
Other Aliases: RP11-536C5.8, HMAT1, HUMMAT1H, MAT1, MAT1H, PEA-15, PED
Other Designations: 15 kDa phosphoprotein enriched in astrocytes; Phosphoprotein enriched in astrocytes, 15 kD; astrocytic phosphoprotein PEA-15; homolog of mouse MAT-1 oncogene; phosphoprotein enriched in diabetes
Nucleotide sequence:
   NCBI Reference Sequence: NM_003768.3
   LOCUS: NM_003768
   ACCESSION: NM_003768 NM_013287
   VERSION: NM_003768.3 GI:208431812
   SEQ ID NO: 164
Protein sequence:
   NCBI Reference Sequence: NP_003759.1
   LOCUS NP_003759
   ACCESSION NP_003759 NP_037419
   VERSION: NP_003759.1 GI:4505705
   SEQ ID NO: 165

### PSMA2

Official Symbol: PSMA2
Official Name: proteasome (prosome, macropain) subunit, alpha type, 2
Gene ID: 5683
Organism: Homo sapiens
Other Aliases: HC3, MU, PMSA2, PSC2
Other Designations: macropain subunit C3; multicatalytic endopeptidase complex subunit C3; proteasome component C3; proteasome subunit HC3; proteasome subunit alpha type-2
Nucleotide sequence:
   NCBI Reference Sequence: NM_002787.4
   LOCUS: NM_002787
   ACCESSION: NM_002787
   VERSION: NM_002787.4 GI:156071494
   SEQ ID NO: 166
Protein sequence:
   NCBI Reference Sequence: NP_002778.1
   LOCUS NP_002778
   ACCESSION NP_002778
   VERSION: NP_002778.1 GI:4506181
   SEQ ID NO: 167

### PSME1

Official Symbol: PSME1
Official Name: proteasome (prosome, macropain) activator subunit 1 (PA28 alpha)
Gene ID: 5720
Organism: Homo sapiens
Other Aliases: IFI5111, PA28A, PA28alpha, REGalpha
Other Designations: 11S regulator complex alpha subunit; 11S regulator complex subunit alpha; 29-kD MCP activator subunit; IGUP I-5111; REG-alpha; activator of multicatalytic protease subunit 1; interferon gamma up-regulated I-5111 protein; interferon-gamma IEF SSP 5111; interferon-gamma-inducible protein 5111; proteasome activator 28 subunit alpha; proteasome activator complex subunit 1; proteasome activator subunit-1
Nucleotide sequence: transcript variant 1
   NCBI Reference Sequence: NM_006263.2
   LOCUS: NM_006263
   ACCESSION: NM_006263
   VERSION: NM_006263.2 GI:30581139
   SEQ ID NO: 168
Protein sequence: isoform 1
   NCBI Reference Sequence: NP_006254.1
   LOCUS NP_006254
   ACCESSION NP_006254
   VERSION: NP_006254.1 GI:5453990
   SEQ ID NO: 169
Nucleotide sequence: transcript variant 2
   NCBI Reference Sequence: NM_176783.1
   LOCUS: NM_176783
   ACCESSION: NM_176783
   VERSION: NM_176783.1 GI:30581140
   SEQ ID NO: 170
Protein sequence: isoform 2
   NCBI Reference Sequence: NP_788955.1
   LOCUS NP_788955
   ACCESSION NP_788955
   VERSION: NP_788955.1 GI:30581141
   SEQ ID NO: 171

### PDIA4

Official Symbol: RPL13
Official Name: ribosomal protein L13
Gene ID: 6137
Organism: Homo sapiens
Other Aliases: OK/SW-c1.46, BBC1, D16S444E, D16S44E, L13
Other Designations: 60S ribosomal protein L13; OK/SW-c1.46; breast basic conserved protein 1
Nucleotide sequence: transcript variant 1
   NCBI Reference Sequence: NM_000977.3
   LOCUS: NM_000977
   ACCESSION: NM_000977
   VERSION: NM_000977.3 GI:341604764
   SEQ ID NO: 172
Protein sequence: isoform 1
   NCBI Reference Sequence: NP_000968.2
   LOCUS NP_000968
   ACCESSION NP_000968
   VERSION: NP_000968.2 GI:15431297
   SEQ ID NO: 173
Nucleotide sequence: transcript variant 3
   NCBI Reference Sequence: NM_001243130.1
   LOCUS: NM_001243130
   ACCESSION: NM_001243130
   VERSION: NM_001243130.1 GI:341604767
   SEQ ID NO: 174
Protein sequence: isoform 2
   NCBI Reference Sequence: NP_001230059.1
   LOCUS NP_001230059
   ACCESSION NP_001230059
   VERSION: NP_001230059.1 GI:341604768
   SEQ ID NO: 175
Nucleotide sequence: transcript variant 4
   NCBI Reference Sequence: NM_001243131.1
   LOCUS: NM_001243131
   ACCESSION: NM_001243131
   VERSION: NM_001243131.1 GI:341604769
   SEQ ID NO: 176
Protein sequence: isoform 3
   NCBI Reference Sequence: NP_001230060.1
   LOCUS NP_001230060
   ACCESSION NP_001230060
   VERSION: NP_001230060.1 GI:341604770
   SEQ ID NO: 177
Nucleotide sequence: transcript variant 2
   NCBI Reference Sequence: NM_033251.2
   LOCUS: NM_033251
   ACCESSION: NM_033251
   VERSION: NM_033251.2 GI:341604766
   SEQ ID NO: 178
Protein sequence: isoform 1
   NCBI Reference Sequence: NP_150254.1
   LOCUS NP_150254
   ACCESSION NP_150254
   VERSION: NP_150254.1 GI:15431295
   SEQ ID NO: 179

### RPS15

Official Symbol: RPS15
Official Name: ribosomal protein S15
Gene ID: 6209
Organism: Homo sapiens
Other Aliases: RIG, S15
Other Designations: 40S ribosomal protein S15; homolog of rat insulinoma; insulinoma protein
Nucleotide sequence:
   NCBI Reference Sequence: NM_001018.3
   LOCUS: NM_001018
   ACCESSION: NM_001018 NM_001080831
   VERSION: NM_001018.3 GI:71284430
   SEQ ID NO: 180
Protein sequence:
   NCBI Reference Sequence: NP_001009.1
   LOCUS NP_001009
   ACCESSION NP_001009 NP_001074300
   VERSION: NP_001009.1 GI:4506687
   SEQ ID NO: 181

### SEC61A1

Official Symbol: SEC61 A1
Official Name: Sec61 alpha 1 subunit (S. cerevisiae)
Gene ID: 29927
Organism: Homo sapiens
Other Aliases: HSEC61, SEC61, SEC61A
Other Designations: Sec61 alpha-1; protein transport protein SEC61 alpha subunit; protein transport protein Sec61 subunit alpha; protein transport protein Sec61 subunit alpha isoform 1; sec61 homolog
Nucleotide sequence:
   NCBI Reference Sequence: NM_013336.3
   LOCUS: NM_013336
   ACCESSION: NM_013336 NM_015968
   VERSION: NM_013336.3 GI:60218911
   SEQ ID NO: 182
Protein sequence:
   NCBI Reference Sequence: NP_037468.1
   LOCUS NP_037468
   ACCESSION NP_037468 NP_057052
   VERSION: NP_037468.1 GI:7019415
   SEQ ID NO: 183

### SEPT2

Official Symbol: SEPT2
Official Name: septin 2
Gene ID: 4735
Organism: Homo sapiens
Other Aliases: DIFF6, NEDD5, Pnut13, hNedd5
Other Designations: NEDD-5; neural precursor cell expressed developmentally down-regulated protein 5; neural precursor cell expressed, developmentally down-regulated 5; septin-2
Nucleotide sequence: transcript variant 1
   NCBI Reference Sequence: NM_001008491.1
   LOCUS: NM_001008491
   ACCESSION: NM_001008491
   VERSION: NM_001008491.1 GI:56549635
   SEQ ID NO: 184
Protein sequence:
   NCBI Reference Sequence: NP_001008491.1
   LOCUS NP_001008491
   ACCESSION NP_001008491
   VERSION: NP_001008491.1 GI:56549636
   SEQ ID NO: 185
Nucleotide sequence: transcript variant 3
   NCBI Reference Sequence: NM_001008492.1
   LOCUS: NM_001008492
   ACCESSION: NM_001008492
   VERSION: NM_001008492.1 GI:56549637
   SEQ ID NO: 186
Protein sequence:
   NCBI Reference Sequence: NP_001008492.1
   LOCUS NP_001008492
   ACCESSION NP_001008492
   VERSION: NP_001008492.1 GI:56549638
   SEQ ID NO: 187
Nucleotide sequence: transcript variant 4
   NCBI Reference Sequence: NM_004404.3
   LOCUS: NM_004404
   ACCESSION: NM_004404
   VERSION: NM_004404.3 GI:56550108
   SEQ ID NO: 188
Protein sequence:
   NCBI Reference Sequence: NP_004395.1
   LOCUS NP_004395
   ACCESSION NP_004395
   VERSION: NP_004395.1 GI:4758158
   SEQ ID NO: 189
Nucleotide sequence: transcript variant 2
   NCBI Reference Sequence: NM_006155.1
   LOCUS: NM_006155
   ACCESSION: NM_006155
   VERSION: NM_006155.1 GI:56549639
   SEQ ID NO: 190
Protein sequence:
   NCBI Reference Sequence: NP_006146.1
   LOCUS NP_006146
   ACCESSION NP_006146
   VERSION: NP_006146.1 GI:56549640
   SEQ ID NO: 191

### SERPINB9

Official Symbol: SERPINB9
Official Name: serpin peptidase inhibitor, clade B (ovalbumin), member 9
Gene ID: 5272
Organism: Homo sapiens
Other Aliases: CAP-3, CAP3, PI-9, PI9
Other Designations: cytoplasmic antiproteinase 3; peptidase inhibitor 9; protease inhibitor 9 (ovalbumin type); serine (or cysteine) proteinase inhibitor, clade B (ovalbumin), member 9; serpin B9; serpin peptidase inhibitor, clade B, member 9
Nucleotide sequence:
   NCBI Reference Sequence: NM_004155.5
   LOCUS: NM_0041.55
   ACCESSION: NM_004155
   VERSION: NM_004155.5 GI:380254460
   SEQ ID NO: 192
Protein sequence:
   NCBI Reference Sequence: NP_004146.1
   LOCUS NP_004146
   ACCESSION NP_004146
   VERSION: NP_004146.1 GI:4758906
   SEQ ID NO: 193

### SMC4

Official Symbol: SMC4
Official Name: structural maintenance of chromosomes 4
Gene ID: 10051
Organism: Homo sapiens
Other Aliases: CAP-C, CAPC, SMC-4, SMC4L1, hCAP-C
Other Designations: SMC protein 4; SMC4 structural maintenance of chromosomes 4-like 1; XCAP-C homolog; chromosome-associated polypeptide C; structural maintenance of chromosomes protein 4
Nucleotide sequence: transcript variant 2
   NCBI Reference Sequence: NM_001002800.1
   LOCUS: NM_001002800
   ACCESSION: NM_001002800
   VERSION: NM_001002800.1 GI:50658062
   SEQ ID NO: 194
Protein sequence:
   NCBI Reference Sequence: NP_001002800.1
   LOCUS NP_001002800
   ACCESSION NP_001002800
   VERSION: NP_001002800.1 GI:50658063
   SEQ ID NO: 195
Nucleotide sequence: transcript variant 1
   NCBI Reference Sequence: NM_005496.3
   LOCUS: NM_005496
   ACCESSION: NM_005496
   VERSION: NM_005496.3 GI:50658064
   SEQ ID NO: 196
Protein sequence:
   NCBI Reference Sequence: NP_005487.3
   LOCUS NP_005487
   ACCESSION NP_005487
   VERSION: NP_005487.3 GI:50658065
   SEQ ID NO: 197

### SPTAN1

Official Symbol: SPTAN1
Official Name: spectrin, alpha, non-erythrocytic 1
Gene ID: 6709
Organism: Homo sapiens
Other Aliases: EIEE5, NEAS, SPTA2
Other Designations: alpha-II spectrin; alpha-fodrin; fodrin alpha chain; spectrin alpha chain, non-erythrocytic 1; spectrin, non-erythroid alpha chain; spectrin, non-erythroid alpha subunit
Nucleotide sequence: transcript variant 1
   NCBI Reference Sequence: NM_001130438.2
   LOCUS: NM_001130438
   ACCESSION: NM_001130438
   VERSION: NM_001130438.2 GI:306966130
   SEQ ID NO: 198
Protein sequence: isoform 1
   NCBI Reference Sequence: NP_001123910.1
   LOCUS NP_001123910
   ACCESSION NP_001123910
   VERSION: NP_001123910.1 GI:194595509
   SEQ ID NO: 199
Nucleotide sequence: transcript variant 3
   NCBI Reference Sequence: NM_001195532.1
   LOCUS: NM_001195532
   ACCESSION: NM_001195532
   VERSION: NM_001195532.1 GI:306966131
   SEQ ID NO: 200
Protein sequence: isoform 3
   NCBI Reference Sequence: NP_001182461.1
   LOCUS NP_001182461
   ACCESSION NP_001182461
   VERSION: NP_001182461.1 GI:306966132
   SEQ ID NO: 201
Nucleotide sequence: transcript variant 2
   NCBI Reference Sequence: NM_003127.3
   LOCUS: NM_003127
   ACCESSION: NM_003127
   VERSION: NM_003127.3 GI:306966129
   SEQ ID NO: 202
Protein sequence: isoform 2
   NCBI Reference Sequence: NP_003118.2
   LOCUS NP_003118
   ACCESSION NP_003118
   VERSION: NP_003118.2 GI:154759259
   SEQ ID NO: 203

### STX6

Official Symbol: STX6
Official Name: syntaxin 6
Gene ID: 10228
Organism: Homo sapiens
Other Aliases: N/A
Other Designations: ntaxin-6
Nucleotide sequence:
   NCBI Reference Sequence: NM_005819.4
   LOCUS: NM_005819
   ACCESSION: NM_005819
   VERSION: NM_005819.4 GI:58294156
   SEQ ID NO: 204
Protein sequence:
   NCBI Reference Sequence: NP_005810.1
   LOCUS NP_005810
   ACCESSION NP_005810
   VERSION: NP_005810.1 GI:5032131
   SEQ ID NO: 205

### TJP2

Official Symbol: TJP2
Official Name: tight junction protein 2
Gene ID: 9414
Organism: Homo sapiens
Other Aliases: RP11-16N10.1, C9DUPq21.11, DFNA51, DUP9q21.11, X104, ZO2
Other Designations: Friedreich ataxia region gene X104 (tight junction protein ZO-2); tight junction protein ZO-2; zona occludens 2; zonula occludens protein 2
Nucleotide sequence: transcript variant 5
   NCBI Reference Sequence: NM_001170414.2
   LOCUS: NM_001170414
   ACCESSION: NM_001170414
   VERSION: NM_001170414.2 GI:358679293
   SEQ ID NO: 206
Protein sequence: isoform 5
   NCBI Reference Sequence: NP_001163885.1
   LOCUS NP_001163885
   ACCESSION NP_001163885
   VERSION: NP_001163885.1 GI:282165800
   SEQ ID NO: 207
Nucleotide sequence: transcript variant 4
   NCBI Reference Sequence: NM_001170415.1
   LOCUS: NM_001170415
   ACCESSION: NM_001170415
   VERSION: NM_001170415.1 GI:282165803
   SEQ ID NO: 208
Protein sequence: isoform 4
   NCBI Reference Sequence: NP_001163886.1
   LOCUS NP_001163886
   ACCESSION NP_001163886
   VERSION: NP_007163886.1 GI:282165804
   SEQ ID NO: 209
Nucleotide sequence: transcript variant 3
   NCBI Reference Sequence: NM_001170416.1
   LOCUS: NM_001170416
   ACCESSION: NM_001170416
   VERSION: NM_001170416.1 GI:282165809
   SEQ ID NO: 210
Protein sequence: isoform 3
   NCBI Reference Sequence: NP_001163887.1
   LOCUS NP_001163887
   ACCESSION NP_001163887
   VERSION: NP_001163887.1 GI:282165810
   SEQ ID NO: 211
Nucleotide sequence: transcript variant 6
   NCBI Reference Sequence: NM_001170630.1
   LOCUS: NM_001170630
   ACCESSION: NM_001170630
   VERSION: NM_001170630.1 GI:282165705
   SEQ ID NO: 212
Protein sequence: isoform 6
   NCBI Reference Sequence: NP_001164101.1
   LOCUS NP_001164101
   ACCESSION NP_001164101
   VERSION: NP_001164101.1 GI:282165706
   SEQ ID NO: 213
Nucleotide sequence: transcript variant 1
   NCBI Reference Sequence: NM_004817.3
   LOCUS: NM_004817
   ACCESSION: NM_004817
   VERSION: NM_004817.3 GI:282165795
   SEQ ID NO: 214
Protein sequence: isoform 1
   NCBI Reference Sequence: NP_004808.2
   LOCUS NP_004808
   ACCESSION NP_004808
   VERSION: NP_004808.2 GI:42518070
   SEQ ID NO: 215
Nucleotide sequence: transcript variant 2
   NCBI Reference Sequence: NM_201629.3
   LOCUS: NM_201629
   ACCESSION: NM_201629
   VERSION: NM_201629.3 GI:318067950
   SEQ ID NO: 216
Protein sequence: isoform 2
   NCBI Reference Sequence: NP_963923.1
   LOCUS NP_963923
   ACCESSION NP_963923
   VERSION: NP_963923.1 GI:42518065
   SEQ ID NO: 217

### TPM4

Official Symbol: TPM4
Official Name: tropomyosin 4
Gene ID: 7171
Organism: Homo sapiens
Other Aliases: N/A
Other Designations: TM30p1; tropomyosin alpha-4 chain; tropomyosin-4
Nucleotide sequence: transcript variant 1
   NCBI Reference Sequence: NM_001145160.1
   LOCUS: NM_001145160
   ACCESSION: NM_00114.5160
   VERSION: NM_001145160.1 GI:223555974
   SEQ ID NO: 218
Protein sequence: isoform 1
   NCBI Reference Sequence: NP_001138632.1
   LOCUS NP_001138632
   ACCESSION NP_001138632
   VERSION: NP_001138632.1 GI:223555975
   SEQ ID NO: 219
Nucleotide sequence: transcript variant 2
   NCBI Reference Sequence: NM_003290.2
   LOCUS: NM_003290
   ACCESSION: NM_003290
   VERSION: NM_003290.2 GI:223555973
   SEQ ID NO: 220
Protein sequence: isoform 2
   NCBI Reference Sequence: NP_003281.1
   LOCUS NP_003281
   ACCESSION NP_003281
   VERSION: NP_003281.1 GI:4507651
   SEQ ID NO: 221

### TSN

Official Symbol: TSN
Official Name: translin
Gene ID: 7247
Organism: Homo sapiens
Other Aliases: BCLF-1, C3PO, RCHF1, REHF-1, TBRBP, TRSLN
Other Designations: component 3 of promoter of RISC; recombination hotspot associated factor; recombination hotspot-binding protein; testis brain-RNA binding protein
Nucleotide sequence: transcript variant 2
   NCBI Reference Sequence: NM_001261401.1
   LOCUS: NM_001261401
   ACCESSION: NM_001261401
   VERSION: NM_001261401.1 GI:386869379
   SEQ ID NO: 222
Protein sequence: isoform 2
   NCBI Reference Sequence: NP_001248330.1
   LOCUS NP_001248330
   ACCESSION NP_001248330
   VERSION: NP_001248330.1 GI:386869380
   SEQ ID NO: 223
Nucleotide sequence: transcript variant 1
   NCBI Reference Sequence: NM_004622.2
   LOCUS: NM_004622
   ACCESSION: NM_004622
   VERSION: NM_004622.2 GI:20302160
   SEQ ID NO: 224
Protein sequence: isoform 1
   NCBI Reference Sequence: NP_004613.1
   LOCUS NP_004613
   ACCESSION NP_004613
   VERSION: NP_004613.1 GI:4759270
   SEQ ID NO: 225

### TUBA4A

Official Symbol: TUBA4A
Official Name: tubulin, alpha 4a
Gene ID: 7277
Organism: Homo sapiens
Other Aliases: H2-ALPHA, TUBA1
Other Designations: tubulin H2-alpha; tubulin alpha-1 chain; tubulin alpha-4A chain; tubulin, alpha 1 (testis specific)
Nucleotide sequence:
   NCBI Reference Sequence: NM_006000.1
   LOCUS: NM_006000
   ACCESSION: NM_006000
   VERSION: NM_006000.1 GI:17921988
   SEQ ID NO: 226
Protein sequence:
   NCBI Reference Sequence: NP_005991.1
   LOCUS NP_005991
   ACCESSION NP_005991
   VERSION: NP_005991.1 GI:17921989
   SEQ ID NO: 227

### TXNDC5

Official Symbol: TXNDC5
Official Name: thioredoxin domain containing 5 (endoplasmic reticulum)
Gene ID: 81567
Organism: Homo sapiens
Other Aliases: RP1-126E20.1, ENDOPDI, ERP46, HCC-2, PDIA15, STRF8, UNQ364
Other Designations: ER protein 46; endoplasmic reticulum protein ERp46; endoplasmic reticulum resident protein 46; endothelial protein disulphide isomerase; protein disulfide isomerase family A, member 15; thioredoxin domain-containing protein 5; thioredoxin related protein; thioredoxin-like protein p46
Nucleotide sequence: transcript variant 3
   NCBI Reference Sequence: NM_001145549.2
   LOCUS: NM_001145549
   ACCESSION: NM_00114.5549
   VERSION: NM_001145549.2 GI:313482855
   SEQ ID NO: 228
Protein sequence: isoform 3
   NCBI Reference Sequence: NP_001139021.1
   LOCUS NP_001139021
   ACCESSION NP_001139021
   VERSION: NP_001139021.1 GI:224493972
   SEQ ID NO: 229
Nucleotide sequence: transcript variant 1
   NCBI Reference Sequence: NM_030810.3
   LOCUS: NM_030810
   ACCESSION: NM_030810
   VERSION: NM_030810.3 GI:313482856
   SEQ ID NO: 230
Protein sequence: isoform 1 precursor
   NCBI Reference Sequence: NP_110437.2
   LOCUS NP_110437
   ACCESSION NP_110437
   VERSION: NP_110437.2 GI:42794771
   SEQ ID NO: 231

### TXNL1

Official Symbol: TXNL1
Official Name: thioredoxin-like 1
Gene ID: 9352
Organism: Homo sapiens
Other Aliases: TRP32, TXL-1, TXNL, Txl
Other Designations: 32 kDa thioredoxin-related protein; thioredoxin-like protein 1; thioredoxin-related 32 kDa protein; thioredoxin-related protein 1
Nucleotide sequence: transcript variant 1
   NCBI Reference Sequence: NM_004786.2
   LOCUS: NM_004786
   ACCESSION: NM_004786
   VERSION: NM_004786.2 GI:215422360
   SEQ ID NO: 232
Protein sequence:
   NCBI Reference Sequence: NP_004777.1
   LOCUS NP_004777
   ACCESSION NP_004777
   VERSION: NP_004777.1 GI:4759274
   SEQ ID NO: 233

### VIM

Official Symbol: VIM
Official Name: vimentin
Gene ID: 431
Organism: Homo sapiens
Other Aliases: RP11-124N14.1
Other Designations: N/A
Nucleotide sequence:
   NCBI Reference Sequence: NM_003380.3
   LOCUS: NM_003380
   ACCESSION: NM_003380
   VERSION: NM_003380.3 GI:240849334
   SEQ ID NO: 234
Protein sequence:
   NCBI Reference Sequence: NP_003371.2
   LOCUS NP_003371
   ACCESSION NP_003371
   VERSION: NP_003371.2 GI:62414289
   SEQ ID NO: 235

### YWHAG

Official Symbol: YWHAG
Official Name: tyrosine 3-monooxygenase/tryptophan 5-monooxygenase activation protein, gamma polypeptide
Gene ID: 7532
Organism: Homo sapiens
Other Aliases: 14-3-3GAMMA
Other Designations: 14-3-3 gamma; 14-3-3 protein gamma; KCIP-1; protein kinase C inhibitor protein 1
Nucleotide sequence:
   NCBI Reference Sequence: NM_012479.3
   LOCUS: NM_012479
   ACCESSION: NM_012479
   VERSION: NM_012479.3 GI:194733744
   SEQ ID NO: 236
Protein sequence:
   NCBI Reference Sequence: NP_036611.2
   LOCUS NP_036611
   ACCESSION NP_036611
   VERSION: NP_036611.2 GI:21464101
   SEQ ID NO: 237

### ZNF207

Official Symbol: ZNF207
Official Name: zinc finger protein 207
Gene ID: 7756
Organism: Homo sapiens
Other Aliases: N/A
Other Designations: N/A
Nucleotide sequence: transcript variant 2
   NCBI Reference Sequence: NM_001032293.2
   LOCUS: NM_001032293
   ACCESSION: NM_001032293
   VERSION: NM_001032293.2 GI:148839356
   SEQ ID NO: 238
Protein sequence: isoform b
   NCBI Reference Sequence: NP_001027464.1
   LOCUS NP_001027464
   ACCESSION NP_001027464
   VERSION: NP_001027464.1 GI:73808090
   SEQ ID NO: 239
Nucleotide sequence: transcript variant 3
   NCBI Reference Sequence: NM_001098507.1
   LOCUS: NM_001098507
   ACCESSION: NM_001098507
   VERSION: NM_001098507 0.1 GI:148612834
   SEQ ID NO: 240
Protein sequence: isoform c
   NCBI Reference Sequence: NP_001091977.1
   LOCUS NP_001091977
   ACCESSION NP_001091977
   VERSION: NP_001091977.1 GI:148612835
   SEQ ID NO: 241
Nucleotide sequence: transcript variant 1
   NCBI Reference Sequence: NM_003457.3
   LOCUS: NM_003457
   ACCESSION: NM_003457
   VERSION: NM_003457.3 GI:148839312
   SEQ ID NO: 242
Protein sequence: isoform a
   NCBI Reference Sequence: NP_003448.1
   LOCUS NP_003448
   ACCESSION NP_003448
   VERSION: NP_003448.1 GI:4508017
   SEQ ID NO: 243

### VI. Diagnostic/Prognostic Uses of the Invention

The invention provides methods for diagnosing a pervasive developmental disorder in a subject, such as, without limitation, autism or Alzheimer's disease. The invention further provides methods for prognosing whether a subject is predisposed to developing a pervasive developmental disorder, e.g., autism or Alzheimer's disease. The invention further provides methods for prognosing response of a pervasive developmental disorder, such as, without limitation, autism or Alzheimer's disease, to a therapeutic treatment. These methods involve the markers of the invention, identified herein and listed in Tables 2-6.

In some embodiments of the present invention, one or more biomarkers is used in connection with the methods of the present invention. As used herein, the term “one or more biomarkers” is intended to mean that at least one biomarker in a disclosed list of biomarkers is assayed and, in various embodiments, more than one biomarker set forth in the list may be assayed, such as two, three, four, five, six, seven, eight, nine, ten, fifteen, twenty, twenty five, thirty, thirty five, forty, forty five, fifty, fifty five, sixty, sixty five, more than sixty five, or all the biomarkers in the list may be assayed. In one embodiment, a panel of biomarkers is used in connection with the methods of the present invention, such that the panel of biomarkers comprises two, three, four, five, six, seven, eight, nine, ten, fifteen, twenty, twenty five, thirty, thirty five, forty, forty five, fifty, fifty five, sixty, sixty five, more than sixty five, or all the biomarkers in the list. In one embodiment, two or more, three or more, four or more, five or more, six or more, seven of more, eight or more, nine or more, ten or more, fifteen or more, twenty or more, twenty five or more, thirty or more, thirty five or more, forty or more, forty five or more, sixty or more, sixty five or more, or all of the biomarkers in the list, are used in connection with the methods of the present invention.

Any suitable analytical method, can be utilized in the methods of the invention to assess (directly or indirectly) the level of expression of a biomarker in a sample. In an embodiment, a difference is observed between the level of expression of a biomarker, as compared to the control level of expression of the biomarker. In one embodiment, the difference is greater than the limit of detection of the method for determining the expression level of the biomarker. In further embodiments, the difference is greater than or equal to the standard error of the assessment method, e.g., the difference is at least about 2-, about 3-, about 4-, about 5-, about 6-, about 7-, about 8-, about 9-, about 10-, about 15-, about 20-, about 25-, about 100-, about 500- or about 1000-fold greater than the standard error of the assessment method. In an embodiment, the level of expression of the biomarker in a sample as compared to a control level of expression is assessed using parametric or nonparametric descriptive statistics, comparisons, regression analyses, and the like.

In an embodiment, a difference in the level of expression of the biomarker in the sample derived from the subject is detected relative to the control, and the difference is about 5%, about 10%, about 15%, about 20%, about 25%, about 30%, about 40%, about 50%, about 60%, about 70%, about 80%, or about 90% more or less than the expression level of the biomarker in the control or normal sample.

In an embodiment, a difference in the level of expression of the biomarker in the sample derived from the subject is detected relative to the control, and the difference is about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100 fold more or less than the expression level of the biomarker in the control or normal sample.

In embodiments where more than one marker is detected, the differences in expression may be different for each marker, or all of markers may have an equivalent minimum level of modulation, e.g., each of the markers detected is at least about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, or about 100 fold up-modulated or down-modulated as compared to the expression level of the respective biomarker in the control or normal sample.

The level of expression of a biomarker, for example one or more markers in Tables 2-6, in a sample obtained from a subject may be assayed by any of a wide variety of techniques and methods, which transform the biomarker within the sample into a moiety that can be detected and/or quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, immunoblotting, Western blotting, Northern blotting, electron microscopy, mass spectrometry, e.g., MALDI-TOF and SELDI-TOF, immunoprecipitations, immunofluorescence, immunohistochemistry, enzyme linked immunosorbent assays (ELISAs), e.g., amplified ELISA, quantitative blood based assays, e.g., serum ELISA, quantitative urine based assays, flow cytometry, Southern hybridizations, array analysis, and the like, and combinations or sub-combinations thereof.

In one embodiment, the level of expression of the biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays (Melton et al., Nuc. Acids Res. 12:7035), Northern blotting and in situ hybridization. Other suitable systems for mRNA sample analysis include microarray analysis (e.g., using Affymetrix's microarray system or Illumina's BeadArray Technology).

In one embodiment, the level of expression of the biomarker is determined using a nucleic acid probe. The term “probe”, as used herein, refers to any molecule that is capable of selectively binding to a specific biomarker. Probes can be synthesized by one of skill in the art, or derived from appropriate biological preparations. Probes can be specifically designed to be labeled, by addition or incorporation of a label. Examples of molecules that can be utilized as probes include, but are not limited to, RNA, DNA, proteins, antibodies, and organic molecules.

As indicated above, isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to the biomarker mRNA. The nucleic acid probe can be, for example, a full-length cDNA, or a portion thereof, such as an oligonucleotide of at least about 7, 10, 15, 20, 25, 30, 35, 40, 45, 50, 100, 250 or about 500 nucleotides in length and sufficient to specifically hybridize under appropriate hybridization conditions to the biomarker genomic DNA. In a particular embodiment, the probe will bind the biomarker genomic DNA under stringent conditions. Such stringent conditions, for example, hybridization in 6× sodium chloride/sodium citrate (SSC) at about 45° C., followed by one or more washes in 0.2×SSC, 0.1% SDS at 50-65° C., are known to those skilled in the art and can be found in Current Protocols in Molecular Biology, Ausubel et al., eds., John Wiley & Sons, Inc. (1995), sections 2, 4, and 6, the teachings of which are hereby incorporated by reference herein. Additional stringent conditions can be found in Molecular Cloning: A Laboratory Manual, Sambrook et al., Cold Spring Harbor Press, Cold Spring Harbor, N.Y. (1989), chapters 7, 9, and 11, the teachings of which are hereby incorporated by reference herein.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface, for example, in an Affymetrix gene chip array, and the probe(s) are contacted with mRNA. A skilled artisan can readily adapt mRNA detection methods for use in determining the level of the biomarker mRNA.

The level of expression of the biomarker in a sample can also be determined using methods that involve the use of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules. These approaches are especially useful for the detection of nucleic acid molecules if such molecules are present in very low numbers. In particular aspects of the invention, the level of expression of the biomarker is determined by quantitative fluorogenic RT-PCR (e.g., the TaqMan™ System). Such methods typically utilize pairs of oligonucleotide primers that are specific for the biomarker. Methods for designing oligonucleotide primers specific for a known sequence are well known in the art.

The expression levels of biomarker mRNA can be monitored using a membrane blot (such as used in hybridization analysis such as Northern, Southern, dot, and the like), or microwells, sample tubes, gels, beads or fibers (or any solid support comprising bound nucleic acids). See, for example, U.S. Pat. Nos. 5,770,722; 5,874,219; 5,744,305; 5,677,195; and 5,445,934, the entire contents of which as they relate to these assays are incorporated herein by reference. The determination of biomarker expression level may also comprise using nucleic acid probes in solution.

In one embodiment of the invention, microarrays are used to detect the level of expression of a biomarker. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, e.g., U.S. Pat. Nos. 6,040,138; 5,800,992; 6,020,135; 6,033,860; and 6,344,316, the entire contents of which as they relate to these assays are incorporated herein by reference. High-density oligonucleotide arrays are particularly useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other known methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting.

Proteins from samples can be isolated using a variety of techniques, including those well known to those of skill in the art. The protein isolation methods employed can, for example, be those described in Harlow and Lane (Harlow and Lane, 1988, Antibodies: A Laboratory Manual, Cold Spring Harbor Laboratory Press, Cold Spring Harbor, New York).

In one embodiment, antibodies, or antibody fragments, are used in methods such as Western blots or immunofluorescence techniques to detect the expressed proteins. Antibodies for determining the expression of the biomarkers of the invention are commercially available.

The antibody or protein can be immobilized on a solid support for Western blots and immunofluorescence techniques. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Other standard methods include immunoassay techniques which are well known to one of ordinary skill in the art and may be found in Principles And Practice Of Immunoassay, 2nd Edition, Price and Newman, eds., MacMillan (1997) and Antibodies, A Laboratory Manual, Harlow and Lane, eds., Cold Spring Harbor Laboratory, Ch. 9 (1988).

In one embodiment of the invention, proteomic methods, e.g., mass spectrometry, are used. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., the biomarker) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis biomarkers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to determine the expression level of a biomarker at the protein level.

Furthermore, in vivo techniques for determination of the expression level of the biomarker include introducing into a subject a labeled antibody directed against the biomarker, which binds to and transforms the biomarker into a detectable molecule. As discussed above, the presence, level, or even location of the detectable biomarker in a subject may be detected by standard imaging techniques.

In general, where a difference in the level of expression of a biomarker and the control is to be detected, it is preferable that the difference between the level of expression of the biomarker in a sample from a subject having a pervasive developmental disorder (e.g., autism or Alzheimer's disease), and the amount of the biomarker in a control sample, is as great as possible. Although this difference can be as small as the limit of detection of the method for determining the level of expression, it is preferred that the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold greater than the standard error of the assessment method.

Any suitable sample obtained from a subject having a pervasive developmental disorder (e.g., autism or Alzheimer's disease) may be used to assess the level of expression, including a lack of expression, of the biomarker, for example one or more markers in Tables 2-6. For example, the sample may be any fluid or component thereof, such as a fraction or extract, e.g., blood, plasma, lymph, synovial fluid, cystic fluid, urine, nipple aspirates, or fluids collected from a biopsy, amniotic fluid, aqueous humor, vitreous humor, bile, blood, breast milk, cerebrospinal fluid, cerumen, chyle, cystic fluid, endolymph, feces, gastric acid, gastric juice, mucus, pericardial fluid, perilymph, peritoneal fluid, plasma, pleural fluid, pus, saliva, sebum, semen, sweat, serum, sputum, synovial fluid, joint tissue or fluid, tears, or vaginal secretions obtained from the subject. In a typical situation, the fluid may be blood, or a component thereof, obtained from the subject, including whole blood or components thereof, including, plasma, serum, and blood cells, such as red blood cells, white blood cells and platelets. In another typical situation, the fluid may be synovial fluid, joint tissue or fluid, or any other sample reflective of a pervasive developmental disorder (e.g., autism or Alzheimer's disease). The sample may also be any tissue or component thereof, connective tissue, lymph tissue or muscle tissue obtained from the subject.

Techniques or methods for obtaining samples from a subject are well known in the art and include, for example, obtaining samples by a mouth swab or a mouth wash; drawing blood; obtaining a biopsy; or obtaining other sample from a subject suffering from a pervasive developmental disorder (e.g., autism or Alzheimer's disease). Isolating components of fluid or tissue samples (e.g., cells or RNA or DNA) may be accomplished using a variety of techniques. After the sample is obtained, it may be further processed.

### Predictive Medicine

The present invention pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the level of expression of one or more marker proteins or nucleic acids, in order to determine whether an individual is at risk of developing a pervasive developmental disorder, such as, without limitation, autism or Alzheimer's disease. Such assays can be used for prognostic or predictive purposes to thereby prophylactically treat an individual prior to the onset of the disorder.

Yet another aspect of the invention pertains to monitoring the influence of agents (e.g., drugs or other compounds administered either to treat a pervasive developmental disorder or symptoms of a pervasive developmental disorder) on the expression or activity of a marker of the invention in clinical trials. These and other agents are described in further detail in the following sections.

A. Diagnostic Assays

An exemplary method for detecting the presence or absence of a marker protein or nucleic acid in a biological sample involves obtaining a biological sample (e.g. a pervasive developmental disorder-associated tissue or body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a marker protein include enzyme linked immunosorbent assays (ELISAs), Western blots, immunoprecipitations and immunofluorescence. In vitro techniques for detection of genomic DNA include Southern hybridizations. In vivo techniques for detection of mRNA include polymerase chain reaction (PCR), Northern hybridizations and in situ hybridizations. Furthermore, in vivo techniques for detection of a marker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of marker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA marker in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-diseased sample, or between samples from different sources.

Alternatively, the expression level can be provided as a relative expression level. To determine a relative expression level of a marker, the level of expression of the marker is determined for 10 or more samples of normal versus pervasive developmental disorder cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cells from a subject that is a normal, healthy control, e.g., cells from a subject that is not afflicted with a pervasive developmental disorder. The choice of the cell source is dependent on the use of the relative expression level. Using expression found in normal tissues as a mean expression score aids in validating whether the marker assayed is specific to a pervasive developmental disorder (versus normal cells). In addition, as more data is accumulated, the mean expression value can be revised, providing improved relative expression values based on accumulated data.

In another embodiment of the present invention, a marker protein is detected. A preferred agent for detecting marker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, New York).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cells express a marker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from pervasive developmental disorder cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a marker protein or nucleic acid in a biological sample. Such kits can be used to determine if a subject is suffering from or is at increased risk of developing a pervasive developmental disorder. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a marker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

The markers of the invention are also useful as pharmacogenomic markers. As used herein, a “pharmacogenomic marker” is an objective biochemical marker whose expression level correlates with a specific clinical drug response or susceptibility in a patient (see, e.g., McLeod et al. (1999) Eur. J. Cancer 35(12): 1650-1652). The presence or quantity of the pharmacogenomic marker expression is related to the predicted response of the patient and more particularly the patient's disorder to therapy with a specific drug or class of drugs. By assessing the presence or quantity of the expression of one or more pharmacogenomic markers in a patient, a drug therapy which is most appropriate for the patient, or which is predicted to have a greater degree of success, may be selected. For example, based on the presence or quantity of RNA or protein encoded by specific tumor markers in a patient, a drug or course of treatment may be selected that is optimized for the treatment of the specific pervasive developmental disorder likely to be present in the patient. The use of pharmacogenomic markers therefore permits selecting or designing the most appropriate treatment for each patient without trying different drugs or regimes.

Another aspect of pharmacogenomics deals with genetic conditions that alters the way the body acts on drugs. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker of the invention.

C. Monitoring Clinical Trials

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for a pervasive developmental disorder. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly. For example, increased expression of the marker gene(s) during the course of treatment may indicate ineffective dosage and the desirability of increasing the dosage. Conversely, decreased expression of the marker gene(s) may indicate efficacious treatment and no need to change dosage.

D. Arrays

The invention also includes an array comprising a marker of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a pervasive developmental disorder, progression of a pervasive developmental disorder, and processes, such a cellular transformation associated with a pervasive developmental disorder.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

### VII. Methods for Obtaining Samples

Samples useful in the methods of the invention include any tissue, cell, biopsy, or bodily fluid sample that expresses a marker of the invention. In one embodiment, a sample may be a tissue, a cell, whole blood, serum, plasma, buccal scrape, saliva, cerebrospinal fluid, urine, stool, or bronchioalveolar lavage. In one embodiment, the tissue sample is a pervasive developmental disorder sample, including a brain tissue sample.

Body samples may be obtained from a subject by a variety of techniques known in the art including, for example, by the use of a biopsy or by scraping or swabbing an area or by using a needle to aspirate bodily fluids. Methods for collecting various body samples are well known in the art.

Tissue samples suitable for detecting and quantitating a marker of the invention may be fresh, frozen, or fixed according to methods known to one of skill in the art. Suitable tissue samples are preferably sectioned and placed on a microscope slide for further analyses. Alternatively, solid samples, i.e., tissue samples, may be solubilized and/or homogenized and subsequently analyzed as soluble extracts.

In one embodiment, a freshly obtained biopsy sample is frozen using, for example, liquid nitrogen or difluorodichloromethane. The frozen sample is mounted for sectioning using, for example, OCT, and serially sectioned in a cryostat. The serial sections are collected on a glass microscope slide. For immunohistochemical staining the slides may be coated with, for example, chrome-alum, gelatine or poly-L-lysine to ensure that the sections stick to the slides. In another embodiment, samples are fixed and embedded prior to sectioning. For example, a tissue sample may be fixed in, for example, formalin, serially dehydrated and embedded in, for example, paraffin.

Once the sample is obtained any method known in the art to be suitable for detecting and quantitating a marker of the invention may be used (either at the nucleic acid or at the protein level). Such methods are well known in the art and include but are not limited to western blots, northern blots, southern blots, immunohistochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods. In particular embodiments, the expression of a marker of the invention is detected on a protein level using, for example, antibodies that specifically bind these proteins.

Samples may need to be modified in order to make a marker of the invention accessible to antibody binding. In a particular aspect of the immunocytochemistry or immunohistochemistry methods, slides may be transferred to a pretreatment buffer and optionally heated to increase antigen accessibility. Heating of the sample in the pretreatment buffer rapidly disrupts the lipid bi-layer of the cells and makes the antigens (may be the case in fresh specimens, but not typically what occurs in fixed specimens) more accessible for antibody binding. The terms “pretreatment buffer” and “preparation buffer” are used interchangeably herein to refer to a buffer that is used to prepare cytology or histology samples for immunostaining, particularly by increasing the accessibility of a marker of the invention for antibody binding. The pretreatment buffer may comprise a pH-specific salt solution, a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffer may, for example, be a solution of 0.1% to 1% of deoxycholic acid, sodium salt, or a solution of sodium laureth-13-carboxylate (e.g., Sandopan LS) or and ethoxylated anionic complex. In some embodiments, the pretreatment buffer may also be used as a slide storage buffer.

Any method for making marker proteins of the invention more accessible for antibody binding may be used in the practice of the invention, including the antigen retrieval methods known in the art. See, for example, Bibbo, et al. (2002) Acta. Cytol. 46:25-29; Saqi, et al. (2003) Diagn. Cytopathol. 27:365-370; Bibbo, et al. (2003) Anal. Quant. Cytol. Histol. 25:8-11, the entire contents of each of which are incorporated herein by reference.

Following pretreatment to increase marker protein accessibility, samples may be blocked using an appropriate blocking agent, e.g., a peroxidase blocking reagent such as hydrogen peroxide. In some embodiments, the samples may be blocked using a protein blocking reagent to prevent non-specific binding of the antibody. The protein blocking reagent may comprise, for example, purified casein. An antibody, particularly a monoclonal or polyclonal antibody that specifically binds to a marker of the invention is then incubated with the sample. One of skill in the art will appreciate that a more accurate prognosis or diagnosis may be obtained in some cases by detecting multiple epitopes on a marker protein of the invention in a patient sample. Therefore, in particular embodiments, at least two antibodies directed to different epitopes of a marker of the invention are used. Where more than one antibody is used, these antibodies may be added to a single sample sequentially as individual antibody reagents or simultaneously as an antibody cocktail. Alternatively, each individual antibody may be added to a separate sample from the same patient, and the resulting data pooled.

Techniques for detecting antibody binding are well known in the art. Antibody binding to a marker of the invention may be detected through the use of chemical reagents that generate a detectable signal that corresponds to the level of antibody binding and, accordingly, to the level of marker protein expression. In one of the immunohistochemistry or immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include, but are not limited to, horseradish peroxidase (HRP) and alkaline phosphatase (AP).

In one particular immunohistochemistry or immunocytochemistry method of the invention, antibody binding to a marker of the invention is detected through the use of an HRP-labeled polymer that is conjugated to a secondary antibody. Antibody binding can also be detected through the use of a species-specific probe reagent, which binds to monoclonal or polyclonal antibodies, and a polymer conjugated to HRP, which binds to the species specific probe reagent. Slides are stained for antibody binding using any chromagen, e.g., the chromagen 3,3-diaminobenzidine (DAB), and then counterstained with hematoxylin and, optionally, a bluing agent such as ammonium hydroxide or TBS/Tween-20. Other suitable chromagens include, for example, 3-amino-9-ethylcarbazole (AEC). In some aspects of the invention, slides are reviewed microscopically by a cytotechnologist and/or a pathologist to assess cell staining, e.g., fluorescent staining (i.e., marker expression). Alternatively, samples may be reviewed via automated microscopy or by personnel with the assistance of computer software that facilitates the identification of positive staining cells.

Detection of antibody binding can be facilitated by coupling the anti-marker antibodies to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, ¹⁴C, or ³H.

In one embodiment of the invention frozen samples are prepared as described above and subsequently stained with antibodies against a marker of the invention diluted to an appropriate concentration using, for example, Tris-buffered saline (TBS). Primary antibodies can be detected by incubating the slides in biotinylated anti-immunoglobulin. This signal can optionally be amplified and visualized using diaminobenzidine precipitation of the antigen. Furthermore, slides can be optionally counterstained with, for example, hematoxylin, to visualize the cells.

In another embodiment, fixed and embedded samples are stained with antibodies against a marker of the invention and counterstained as described above for frozen sections. In addition, samples may be optionally treated with agents to amplify the signal in order to visualize antibody staining. For example, a peroxidase-catalyzed deposition of biotinyl-tyramide, which in turn is reacted with peroxidase-conjugated streptavidin (Catalyzed Signal Amplification (CSA) System, DAKO, Carpinteria, Calif.) may be used.

Tissue-based assays (i.e., immunohistochemistry) are the preferred methods of detecting and quantitating a marker of the invention. In one embodiment, the presence or absence of a marker of the invention may be determined by immunohistochemistry. In one embodiment, the immunohistochemical analysis uses low concentrations of an anti-marker antibody such that cells lacking the marker do not stain. In another embodiment, the presence or absence of a marker of the invention is determined using an immunohistochemical method that uses high concentrations of an anti-marker antibody such that cells lacking the marker protein stain heavily. Cells that do not stain contain either mutated marker and fail to produce antigenically recognizable marker protein, or are cells in which the pathways that regulate marker levels are dysregulated, resulting in steady state expression of negligible marker protein.

One of skill in the art will recognize that the concentration of a particular antibody used to practice the methods of the invention will vary depending on such factors as time for binding, level of specificity of the antibody for a marker of the invention, and method of sample preparation. Moreover, when multiple antibodies are used, the required concentration may be affected by the order in which the antibodies are applied to the sample, e.g., simultaneously as a cocktail or sequentially as individual antibody reagents. Furthermore, the detection chemistry used to visualize antibody binding to a marker of the invention must also be optimized to produce the desired signal to noise ratio.

In one embodiment of the invention, proteomic methods, e.g., mass spectrometry, are used for detecting and quantitating the marker proteins of the invention. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate the PY-Shc and/or p66-Shc proteins. Mass spectrometric methods are described in, for example, U.S. Pat. Nos. 5,622,824, 5,605,798 and 5,547,835, the entire contents of each of which are incorporated herein by reference.

In other embodiments, the expression of a marker of the invention is detected at the nucleic acid level. Nucleic acid-based techniques for assessing expression are well known in the art and include, for example, determining the level of marker mRNA in a sample from a subject. Many expression detection methods use isolated RNA. Any RNA isolation technique that does not select against the isolation of mRNA can be utilized for the purification of RNA from cells that express a marker of the invention (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to a marker of the invention, for example, a nucleotide transcript and/or protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the marker mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to marker genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of marker mRNA.

An alternative method for determining the level of marker mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, marker expression is assessed by quantitative fluorogenic RT-PCR (i.e., the TaqMan™ System). Such methods typically utilize pairs of oligonucleotide primers that are specific for a marker of the invention. Methods for designing oligonucleotide primers specific for a known sequence are well known in the art.

The expression levels of a marker of the invention may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of marker expression may also comprise using nucleic acid probes in solution.

In one embodiment of the invention, microarrays are used to detect the expression of a marker of the invention. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

The amounts of phosphorylated marker, and/or a mathematical relationship of the amounts of a marker of the invention may be used to calculate the risk of recurrence of a pervasive developmental disorder in a subject being treated for a pervasive developmental disorder, the survival of a subject being treated for a pervasive developmental disorder, whether a pervasive developmental disorder is aggressive, the efficacy of a treatment regimen for treating a pervasive developmental disorder, and the like, using the methods of the invention, which may include methods of regression analysis known to one of skill in the art. For example, suitable regression models include, but are not limited to CART (e.g., Hill, T, and Lewicki, P. (2006) “STATISTICS Methods and Applications” StatSoft, Tulsa, Okla.), Cox (e.g., www.evidence-based-medicine.co.uk), exponential, normal and log normal (e.g., www.obgyn.cam.ac.uk/mrg/statsbook/stsurvan.html), logistic (e.g., www.en.wikipedia.org/wiki/Logistic_regression or http://faculty.chass.ncsu.edu/garson/PA765/logistic.htm), parametric, non-parametric, semi-parametric (e.g., www.socserv.mcmaster.ca/jfox/Books/Companion), linear (e.g., www.en.wikipedia.org/wiki/Linear_regression or http://www.curvefit.com/linear_regression.htm), or additive (e.g., www.en.wikipedia.org/wiki/Generalized_additive_model or http://support.sas.com/rnd/app/da/new/dagam.html).

In one embodiment, a regression analysis includes the amounts of phosphorylated marker. In another embodiment, a regression analysis includes a marker mathematical relationship. In yet another embodiment, a regression analysis of the amounts of phosphorylated marker, and/or a marker mathematical relationship may include additional clinical and/or molecular co-variates. Such clinical co-variates include, but are not limited to, nodal status, tumor stage, tumor grade, tumor size, treatment regime, e.g., chemotherapy and/or radiation therapy, clinical outcome (e.g., relapse, disease-specific survival, therapy failure), and/or clinical outcome as a function of time after diagnosis, time after initiation of therapy, and/or time after completion of treatment.

In another embodiment, the amounts of phosphorylated marker, and/or a mathematical relationship of the amounts of a marker may be used to calculate the risk of recurrence of an oncologic disorder in a subject being treated for an oncologic disorder, the survival of a subject being treated for an oncologic disorder, whether an oncologic disorder is aggressive, the efficacy of a treatment regimen for treating an oncologic disorder, and the like, using the methods of the invention, which may include methods of regression analysis known to one of skill in the art. For example, suitable regression models include, but are not limited to CART (e.g., Hill, T, and Lewicki, P. (2006) “STATISTICS Methods and Applications” StatSoft, Tulsa, Okla.), Cox (e.g., www.evidence-based-medicine.co.uk), exponential, normal and log normal (e.g., www.obgyn.cam.ac.uk/mrg/statsbook/stsurvan.html), logistic (e.g., www.en.wikipedia.org/wiki/Logistic_regression or http://faculty.chass.ncsu.edu/garson/PA765/logistic.htm), parametric, non-parametric, semi-parametric (e.g., www.socserv.mcmaster.ca/jfox/Books/Companion), linear (e.g., www.en.wikipedia.org/wiki/Linear_regression or http://www.curvefit.com/linear_regression.htm), or additive (e.g., www.en.wikipedia.org/wiki/Generalized_additive_model or http://support.sas.com/rnd/app/da/new/dagam.html).

In one embodiment, a regression analysis includes the amounts of phosphorylated marker. In another embodiment, a regression analysis includes a marker mathematical relationship. In yet another embodiment, a regression analysis of the amounts of phosphorylated marker, and/or a marker mathematical relationship may include additional clinical and/or molecular co-variates. Such clinical co-variates include, but are not limited to, nodal status, tumor stage, tumor grade, tumor size, treatment regime, e.g., chemotherapy and/or radiation therapy, clinical outcome (e.g., relapse, disease-specific survival, therapy failure), and/or clinical outcome as a function of time after diagnosis, time after initiation of therapy, and/or time after completion of treatment.

### VIII. Kits

The invention also provides compositions and kits for prognosing a disease or disorder, recurrence of a disorder, or survival of a subject being treated for a disorder (e.g., a pervasive developmental disorder, such as autism and/or Alzheimer's disorder). These kits include one or more of the following: a detectable antibody that specifically binds to a marker of the invention, a detectable nucleic acid that specifically binds to a marker of the invention, reagents for obtaining and/or preparing subject tissue samples for staining, and instructions for use.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention and tissue specific controls/standards.

### IX. Screening Assays

Targets of the invention include, but are not limited to, the genes and proteins described herein. Screening assays useful for identifying modulators of identified markers are described below.

The invention also provides methods (also referred to herein as “screening assays”) for identifying modulators, i.e., candidate or test compounds or agents (e.g., proteins, peptides, peptidomimetics, peptoids, small molecules or other drugs), which modulate the state of the diseased cell by modulating the expression and/or activity of a marker of the invention. Such assays typically comprise a reaction between a marker of the invention and one or more assay components. The other components may be either the test compound itself, or a combination of test compounds and a natural binding partner of a marker of the invention. Compounds identified via assays such as those described herein may be useful, for example, for modulating, e.g., inhibiting, ameliorating, treating, or preventing the disease.

The test compounds used in the screening assays of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

The screening methods of the invention comprise contacting a cell, e.g., a diseased cell, with a test compound and determining the ability of the test compound to modulate the expression and/or activity of a marker of the invention in the cell. The expression and/or activity of a marker of the invention can be determined as described herein.

In another embodiment, the invention provides assays for screening candidate or test compounds which are substrates of a marker of the invention or biologically active portions thereof. In yet another embodiment, the invention provides assays for screening candidate or test compounds which bind to a marker of the invention or biologically active portions thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹³¹I, ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

This invention further pertains to novel agents identified by the above-described screening assays. Accordingly, it is within the scope of this invention to further use an agent identified as described herein in an appropriate animal model. For example, an agent capable of modulating the expression and/or activity of a marker of the invention identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment as described above.

### X. Treatment of Disease States

The present invention provides methods for treating a pervasive developmental disorder, or symptoms of a pervasive developmental disorder, by administering to a subject (e.g., a mammal, e.g., a human) in need thereof one or more of the proteins listed in Tables 2-6. In one embodiment, the pervasive developmental disorder is autism. In one embodiment, the pervasive developmental disorder is Alzheimer's disease. In other embodiments, the pervasive developmental disorder is any one of the disorders described herein.

In one aspect, the invention provides a method for treating, alleviating symptoms of, inhibiting progression of, or preventing a pervasive developmental disorder in a subject, the method comprising administering to the subject in need thereof a therapeutically effective amount of a pharmaceutical composition comprising one or more of the markers listed in Tables 2-6. In one embodiment, the marker is a protein or fragment thereof. In one embodiment, the marker is a nucleic acid, e.g., RNA or DNA, encoding or expressing a protein marker or fragment thereof. The markers suitable for such a method are further described in detail herein.

In another aspect, the invention provides a method for treating, alleviating symptoms of, inhibiting progression of, or preventing a pervasive developmental disorder in a subject, the method comprising administering to the subject in need thereof a therapeutically effective amount of a pharmaceutical composition comprising an agent that modulates expression or activity of one or more of the markers listed in Tables 2-6.

In one embodiment, the agent that modulates expression or activity of the one or more of the markers listed in Tables 2-6 is identified using any one of the screening assays described herein. In one embodiment, the agent inhibits expression or activity of one or more of the markers listed in Tables 2-6. In one embodiment, the agent augments expression or activity of one or more of the markers listed in Tables 2-6.

The invention further provides a method for assessing the efficacy of a treatment regimen for treating a pervasive developmental disorder or symptoms of a pervasive developmental disorder in a subject, the method comprising: (1) determining a level of expression of one or more of the markers listed in Tables 2-6 present in a first biological sample obtained from the subject prior to administering at least a portion of the treatment regimen to the subject, using reagents that transform the markers such that the markers can be detected; (2) determining a level of expression of one or more of the markers listed in Tables 2-6 present in a second biological sample obtained from the subject following administration of at least a portion of the treatment regimen to the subject, using reagents that transform the markers such that the markers can be detected; (3) comparing the level of expression of one or more markers listed in Tables 2-6 present in a first sample obtained from the subject prior to administering at least a portion of the treatment regimen to the subject with the level of expression of the one or more markers present in a second sample obtained from the subject following administration of at least a portion of the treatment regimen; and (4) assessing whether the treatment regimen is efficacious for treating the pervasive developmental disorder or symptoms of the pervasive developmental disorder.

In one embodiment, a modulation in the level of expression of the one or more markers in the second sample as compared to the first sample is an indication that the treatment regimen is efficacious for treating the pervasive developmental disorder or symptoms of the pervasive developmental disorder in the subject. In one embodiment, a similar level of expression of the one or more markers in the second sample as compared to the first sample is an indication that the treatment regimen is non-efficacious for treating the pervasive developmental disorder or symptoms of the pervasive developmental disorder in the subject.

In some embodiments, modulation of the level of expression in the second sample towards normal or control levels of expression, e.g., closer to normal or control levels of expression than that of the levels of expression in the first sample, is an indication that the treatment regimen is efficacious for treating the pervasive developmental disorder or symptoms of the pervasive developmental disorder in the subject.

In one embodiment, the subject is undergoing a treatment for the pervasive developmental disorder. In some embodiments, the method further comprises continuing administration of the treatment regimen to the subject for whom the treatment regimen is determined to be efficacious for treating the pervasive developmental disorder or symptoms of the pervasive developmental disorder, and/or discontinuing administration of the treatment regimen to the subject for whom the treatment regimen is determined to be non-efficacious for treating the pervasive developmental disorder or symptoms of the pervasive developmental disorder.

In another aspect, the invention provides a method of identifying a compound for treating a pervasive developmental disorder or symptoms of pervasive developmental disorders in a subject, the method comprising: (1) contacting a biological sample with a test compound; (2) determining the level of expression and/or activity of one or more markers listed in Tables 2-6 present in the biological sample; (3) comparing the level of expression and/or activity of the one or more markers in the biological sample with that of a control sample not contacted by the test compound; and (4) selecting a test compound that modulates the level of expression and/or activity of the one or more markers in the biological sample, thereby identifying a compound for treating a pervasive developmental disorder or symptoms of a pervasive developmental disorder in a subject.

In one embodiment the biological sample is obtained from a subject suffering from a pervasive developmental disorder or symptoms of a pervasive developmental disorder. In one embodiment the subject is a human. In one embodiment, the biological sample is a tissue or a biological fluid from the subject, e.g., a subject suffering from a pervasive developmental disorder or symptoms of a pervasive developmental disorder. In one embodiment, the biological sample comprises cells, e.g., primary cells from a subject or immortalized cells for use in in vitro assays.

In one embodiment, the test compound up-modulates the expression and/or activity of one or more markers listed in Tables 2-6. In one embodiment, the test compound down-modulates the expression and/or activity of one or markers listed in Tables 2-6. In one embodiment, the test compound modulates the expression and/or activity of one or more markers listed in Tables 2-6 towards, or to a level similar or identical to, the level of expression of a control sample.

In another aspect, the invention provides a method of treating a subject having a pervasive developmental disorder with a treatment regimen, the method comprising the steps of: selecting a subject exhibiting a modulated level of expression of one or more of the markers listed in Tables 2-6 as compared to a level of expression of a control marker in response to the treatment regimen; and administering a therapeutically effective amount of the treatment regimen to the subject.

This invention is further illustrated by the following examples which should not be construed as limiting. The contents of all references and published patents and patent applications cited throughout the application are hereby incorporated by reference.

### EXEMPLIFICATION OF THE INVENTION

This invention is further illustrated by the following examples which should not be construed as limiting. The contents of all references and published patents and patent applications cited throughout the application are hereby incorporated by reference.

### Example 1: Proteins Identified as Uniquely Up or Down Regulated in Autism Vs. Normal Samples

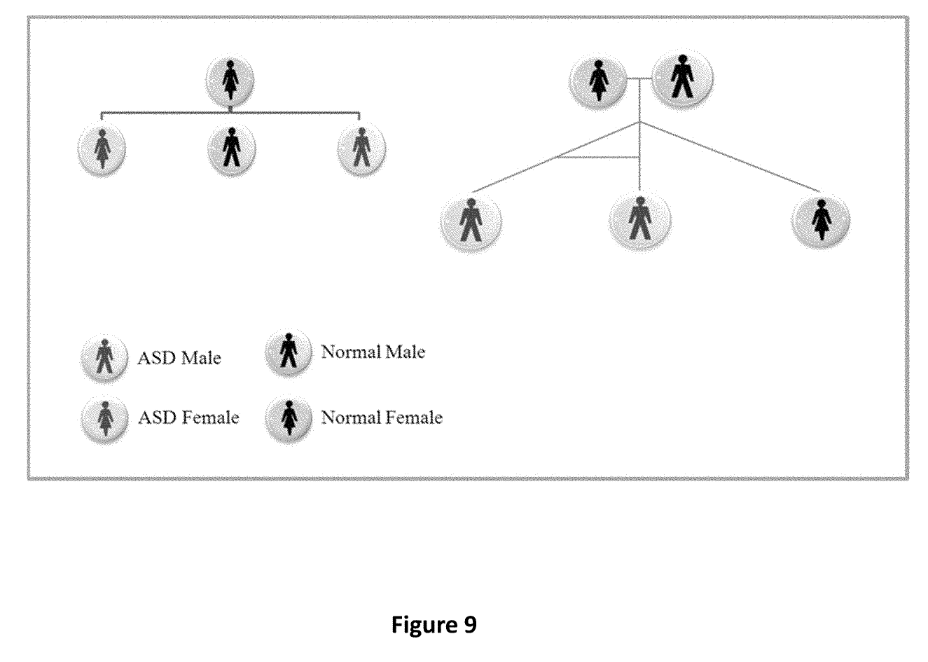

Studies were performed using the above described Platform Technology with lymphoblast cells from autism patients and normal unafflicted parents or siblings of the autism patients to identify proteins which are uniquely upregulated or downregulated in the autism disease state. Lymphoblast cell samples from four autism patients and five unafflicted controls (see FIG. 9) were prepared by using the cell lines obtained from Coriell Cell Repositories (403 Haddon Avenue Camden, N.J. 08103). The results of these studies were analyzed using data processing within the Platform Technology as described above.

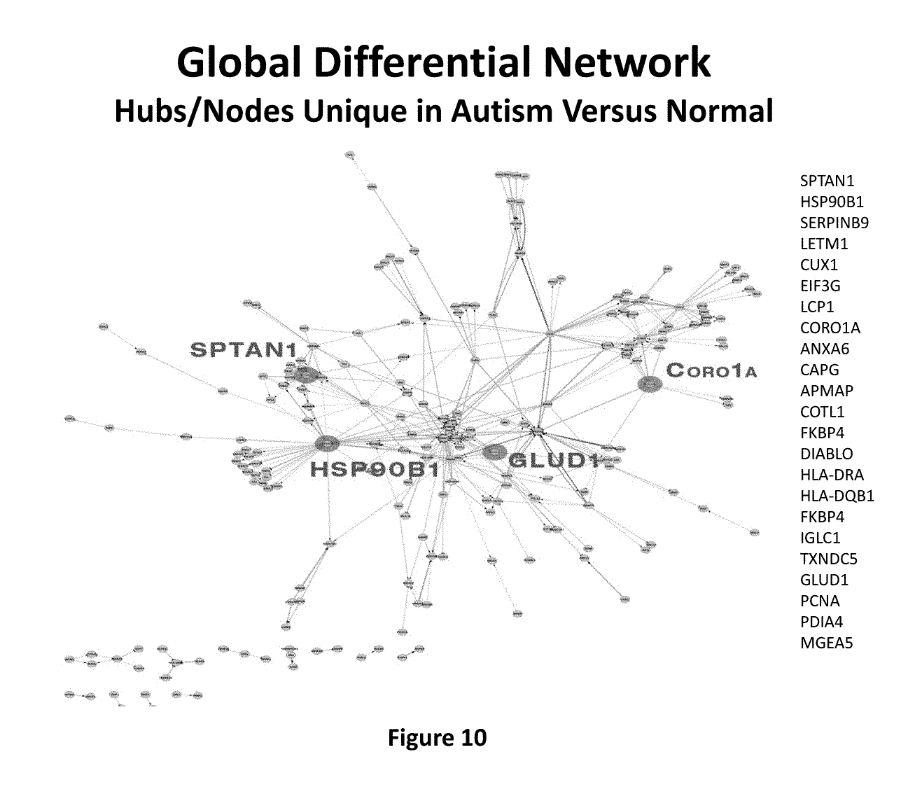

The results of these studies identified proteins such as SPTAN1, HSP90B1, GLUD1, and CORO1A as global differential network hubs/nodes which are uniquely up or down regulated in samples from Autism patients compared to samples from normal unafflicted parents or siblings of the autism patients (see FIG. 10). Moreover, the studies identified the following proteins within the network of SPTAN1, HSP90B1, GLUD1, and CORO1A, as uniquely up or down regulated in samples from Autism patients comparing to samples from normal parents or siblings of the autism patients.

TABLE 2
SPTAN1, HSP90B1, SERPINB9, LETM1, CUX1, EIF3G, LCP1,
CORO1A, ANXA6, CAPG, APMAP, COTL1, FKBP4, DIABLO,
HLA-DRA, HLA-DQB1, FKBP4, IGLC1, TXNDC5, GLUD1, PCNA,
PDIA4, and MGEA5

These results indicated that proteins such as such as SPTAN1, HSP90B1, SERPINB9, LETM1, CUX1, EIF3G, LCP1, CORO1A, ANXA6, CAPG, APMAP, COTL1, FKBP4, DIABLO, HLA-DRA, HLA-DQB1, FKBP4, IGLC1, TXNDC5, GLUD1, PCNA, PDIA4, and MGEA5 can serve as markers for diagnosing a pervasive developmental disorder, e.g., autism, for identifying a predisposition or risk for developing a pervasive developmental disorder, e.g., autism, and as targets useful for developing pharmaceutical treatments of a pervasive developmental disorder, e.g., autism.

Spectrin A2 (SPTAN1) was identified as one of the molecular entities influenced by autism. SPTAN1 is a protein expressed in non-erythrocytic cells, which is also know as “Spectrin A2.” Mutation of SPTAN1 is linked to West Syndrome such as hypomyelination, quadriplegia and development delay. Aberrant spectrin characteristics are evident in brain and lymphoblastic cells of Autism patients. The loci of SPTAN1 is close to the loci of TSC1. Expression of SPTAN1 influences T-cell maturation and CD4/CD8 ratios. SPTAN1 has a characteristic aggregation pattern in T-cell activation.

Coronin 1A (CORO1A) was identified as a hub in autism network. CORO1A is an actin binding protein which is involved in signal transduction, apoptosis, and gene regulation patherways. CORO1A is a key player in T-cell survical activation and migration. Mutation of CORO1A is associated with T-cell egress from thymus resulting in peripheral deficiency. Mutation of CORO1A is associated with severe combined immunodeficiency and ADHD.

GLUD1 is a mitochondrial specific protein which plays a key role in ammonia detoxification. Based on the identification of GLUD1 as being modulated in samples from autism patients, increased ammonia levels observed in autism plasma may be due to mitochondrial dysfunction, e.g., GLUD1 dysfunction. Activity of GLUD1 is influenced by ATP levels.

HSP90B1 is a ER specific heat shock protein which is a GRP member. HSP90B1 is a master chaperone of integrins and is a T & B lymphopoiesis regulator. HSP90B1 interacts with genes reported to be associated with autism.

### Example 2: Molecular Entities Driven by Disease State and Identified as Common to Autism and Alzheimer's Disease

Studies were performed using the above described Platform Technology with lymphoblast cells from autism or Alzheimer's disease patients and from normal, control individuals, e.g., unafflicted parents or siblings of the Autism and/or Alzheimer's patients, to identify proteins which are uniquely upregulated or downregulated as compared to controls and also common to both autism and Alzheimer patients. Lymphoblast cell samples from four autism patients and five unafflicted controls (see FIG. 9), and from four Alzheimer patients and four healthy controls (matching age and gender), were prepared by using the cell lines obtained from Coriell Cell Repositories (403 Haddon Avenue Camden, N.J. 08103). The results of these studies were analyzed using data processing within the Platform Technology as described above.

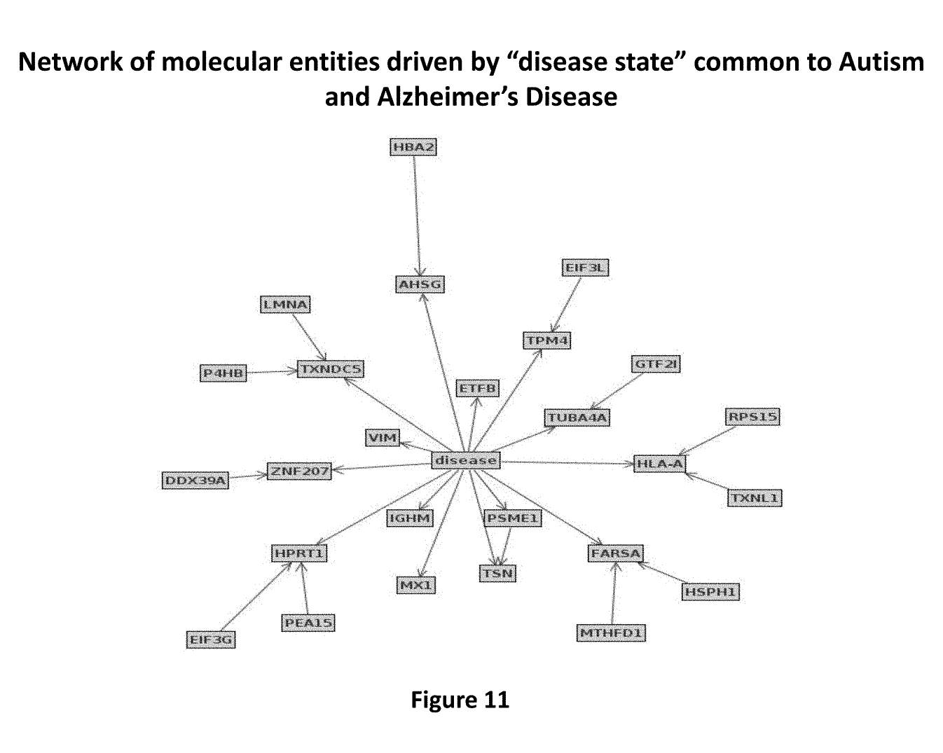

The results of these studies identified that the following proteins were commonly modulated, e.g., upregulated or downregulated, in samples from both Autism and Alzheimer's disease patients as compared to samples from normal, unafflicted individuals (e.g., unafflicted parents or siblings of the autism or Alzheimer's patients). See FIG. 11.

TABLE 3
HBA2, AHSG, LMNA, P4HB, TXNDC5, VIM, DDX39A, ZNF207,
EIF3G, HPRT1, PEA15, IGHM, MX1, ETFB, EIF3L, TPM4, GTF2I,
TUBA4A, RPS15, HLA-A, TXNL1, PSME1, TSN, FARSA, MTHFD1,
and HSPH1

These results indicated that proteins such as such as HBA2, AHSG, LMNA, P4HB, TXNDC5, VIM, DDX39A, ZNF207, EIF3G, HPRT1, PEA15, IGHM, MX1, ETFB, EIF3L, TPM4, GTF2I, TUBA4A, RPS15, HLA-A, TXNL1, PSME1, TSN, FARSA, MTHFD1, and HSPH1 can serve as markers for diagnosing a pervasive developmental disorder, such as autism and/or Alzheimer's disease, for identifying a predisposition or risk for developing a pervasive developmental disorder, e.g., autism and or Alzheimer's disease, and as targets useful for developing pharmaceutical treatment of a pervasive developmental disorder, such as autism and/or Alzheimer's disease.

### Example 3: Novel Autism Spectrum Disorders (ASD) Biomarkers Identified Using the Interrogative Biology Discovery Platform

Applicants have employed herein a novel approach combining the power of cell biology and multi-omics platforms in an Interrogative Discovery Platform Technology in order to identify novel biomarkers for Autism Spectrum disorder, e.g., autism. A cell model system for Autism Spectrum Disorder, and in particular for autism, was developed and employed, which comprised Lymphoblast cell lines obtained from patients used as cell model to represent Autism disorder. These cells were treated with or without the MIMs to capture the pathological proteome changes unique to a pervasive developmental disorder, e.g., autism. A 2D-nanoLC-MSMS workflow was developed to profile and relatively quantify the cellular and secreted peptides/proteins. While only proteomic analysis was carried out in this example, multiple data output may readily be employed and analyzed in the platform technology, including data from flow cytometry, cell-based assays (e.g. mitochondria ATP and ROS assays) and functional genomic platforms (e.g. single-nucleotide polymorphism (SNP) data), to provide insightful biological readout. All data obtained in the present example (i.e., proteomic data) were subjected to a AI based REFS™ informatics platform in an effort to study congruent data trends with in vitro, in vivo, and in silico modeling. By using this process, a molecular fingerprint was developed of a cellular signaling network associated with the disease phenotype, thereby providing insight into the mechanisms that dictate the molecular alterations that lead to disease (e.g., a pervasive development disorder) onset and progression. Using this approach, several novel biomarkers have been identified from the causal network. In addition, using cellular functional readouts such as mitochondrial ATP, bioenergetics, ROS etc., markers that drive pathophysiological cellular behavior were determined. Taken together, the methodologies described herein represent a solid foundation for the identification of biomarkers useful for diagnoses and patient stratification in Autism Spectrum Disorder (ASD).

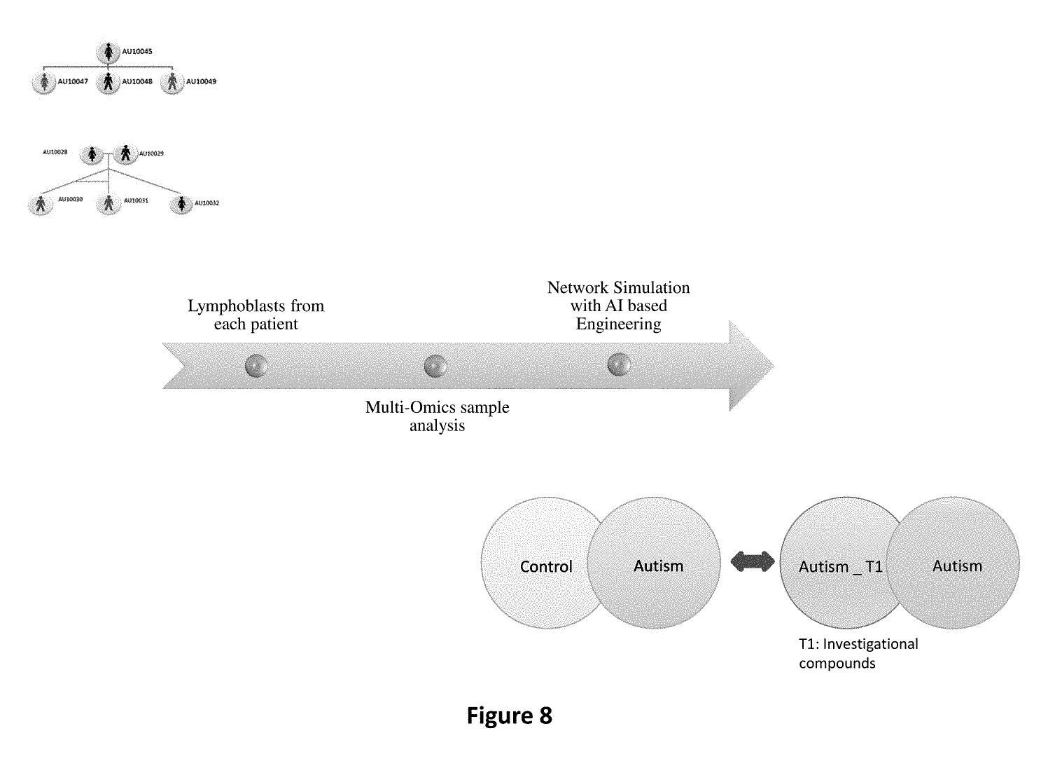

An example of the specific experimental approach employed is depicted in FIG. 8. Briefly, lymphoblasts were sampled from autism patients and normal unafflicted parents or siblings. Lymphoblast cell samples from four autism patients and five unafflicted controls (see FIG. 9) were prepared by using the cell lines obtained from Coriell Cell Repositories (403 Haddon Avenue Camden, N.J. 08103). An Omics analysis, e.g. 2D-nanoLC-MSMS proteomics analysis, was performed on the samples. Multi-Omics sample analysis readout were inputted into the AI based REFS informatics platform as described above. Differential interactome network output has identified biomarkers which are uniquely expressed or modulated/deregulated in the autism disease state.

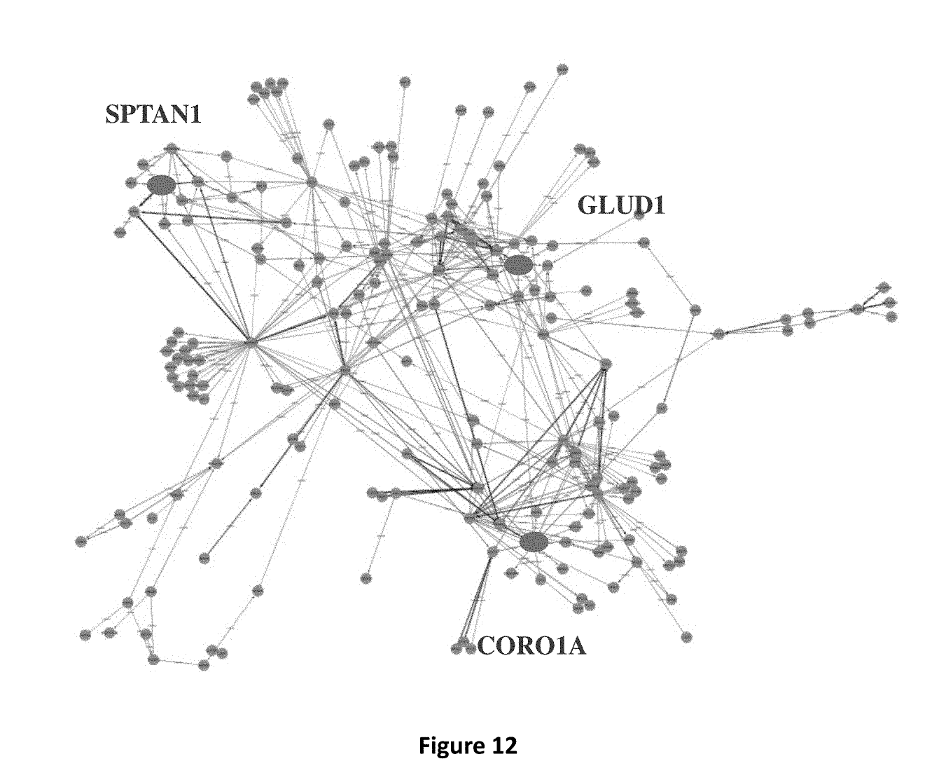

One exemplary simulated differential delta network which compares the autism patients to normal unafflicted parents or siblings is shown in FIG. 12. This differential network is a re-constructed network based exclusively on the data collected, i.e., no previous biological knowledge was used to create the network. In the network, three critical “hubs” or “modulators” of ASD pathophysiology were identified and are highlighted in FIG. 12.

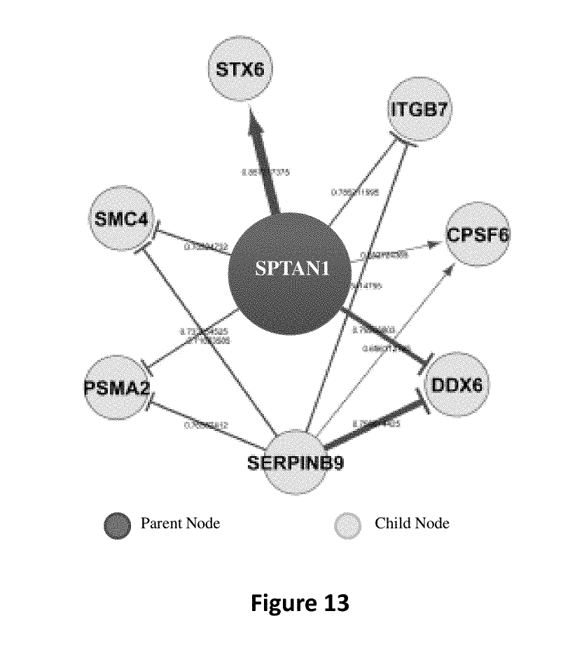

For the first hub (as shown in FIG. 13), the parent node, Spectrin A2 (SPTAN1), plays a role in cell signaling and peripheral nerve myelination. The dominant negative mutation of SPTAN1 causes western syndrome, with cerebral hypomyelination, poor visual attention, spastic quadriplegia, and developmental delay. The characteristic aberrant spectrin was reported in brain and lymphoblast cells. No literature has reported on SPTAN1's role in autism. However, a role for myelination in autism was previously reported. For one of the child nodes, Syntaxin-6 (STX6), there have been no reports linking STX6 to autism. An STX6 mutation was reported to be involved in toxin absorption and to be involved in another neurodegenerative disease, Progressive supranuclear (PSP). Child node Integrin beta 7 (ITGB7) was reported to be differentially expressed in autistic children compared to their normal siblings (see Hu et al. BMC Genomics 2006; Szatmari et al., Nat Genet. 2007). For neighboring node SERPINB9, which shared multiple child nodes with Serpin peptidase inhibitor, clade (SPTAN1), a microarray study reported that down-regulation of this gene expression is associated with autistic patients compared to their normal siblings (Hu et al. Autism Res. 2009).

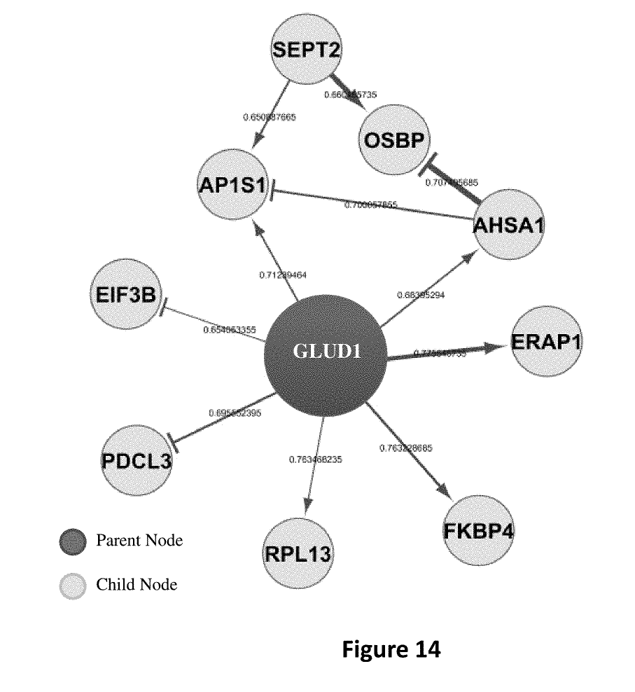

The second hub, Glutamate dehydrogenase 1 (GLUD1), is the parent node shown in FIG. 14. GLUD1 is a mitochondria matrix enzyme and it plays a key role in nitrogen and glutamate metabolism, and in energy homeostasis in the brain. Upregulation of GLUD1 has been reported in autistic children in early onset stage (Gregg et al., Genomics. 2008). Increased ammonia levels in autism plasma are suggested to be due to mitochondrial dysfunction. The child nodes of GLUD1, EIF3B and RPL3, have both been linked to the autistic phenotype by CNV analysis. The upregulation of GLUD1's neighboring node Septin 2 (SEPT2) has also been detected in early onset autism (Gregg et al., Genomics. 2008). GLUD1's child nodes EIF3B and RPL3 are genetically associated to the autistic phenotype by CNV analysis.

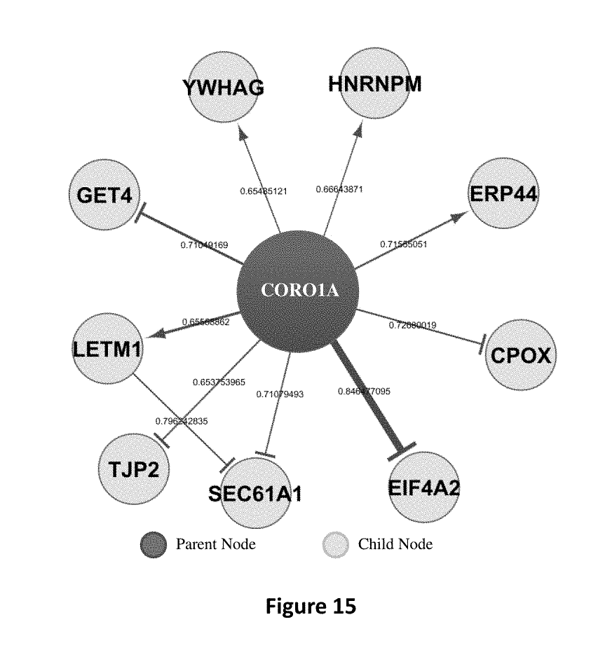

The third hub, Coronin-1A (CORO1A), is the parent node shown in FIG. 15. CORO1A is involved in signal transduction, mitochondria apoptosis, T-cell mediated immunity and gene regulation. Mutation of CORO1A is associated with severe combined immunodeficiency and ADHD. The child node Coproporphyrinogen III oxidase (CPDX) is a mitochondria inner membrane enzyme. CPDX may be associated with mitochondria respiratory chain disorder. Disregulation of CPDX is linked to exaggerated porphyrin excretion as observed among some autistic patients. Urine porphyrin levels are used as the indicator for mercury exposure as urinary porphyrin positively correlates to mercury exposure.

The results of these studies identified including SPTAN1, GLUD1, and CORO1A as global differential network hubs/nodes which are uniquely expressed or modulated/disregulated in samples from Autism patients as compared to samples from normal unafflicted parents or siblings of the autism patients. Moreover, the studies identified the following additional listed in Tables 4-6 below within the network of SPTAN1, GLUD1, and CORO1A, respectively, as uniquely expressed or modulated/disregulated in samples from Autism patients as compared to samples from normal parents or siblings of the autism patients.

TABLE 4
SPTAN1, STX6, ITGB7, CPSF6, DDX6, SERPINB9, PSMA2, SMC4

TABLE 5
GLUD1, SEPT2, OSBP, AHSA1, ERAP1, FKBP4, RPL13, PDCL3,
EIF3B, AP1S1

TABLE 6
CORO1A, YWHAG, HNRNPM, ERP44, CPOX, EIF4A2, SEC61A1,
TJP2, LETM1, GET4

These results indicated that proteins such as SPTAN1, STX6, ITGB7, CPSF6, DDX6, SERPINB9, PSMA2, SMC4, GLUD1, SEPT2, OSBP, AHSA1, ERAP1, FKBP4, RPL13, PDCL3, EIF3B, AP1S1, CORO1A, YWHAG, HNRNPM, ERP44, CPDX, EIF4A2, SEC61A1, TJP2, LETM1, and GET4 can serve as markers for diagnosing a pervasive developmental disorder, e.g., autism or autism spectrum disorder, for identifying a predisposition or risk for developing a pervasive developmental disorder, e.g., autism or Alzheimer's disease, and as targets useful for developing pharmaceutical treatments of a pervasive developmental disorder, e.g., autism or autism spectrum disorder.

In conclusion, the Interrogative Discovery Platform Technology used in this example is exclusively data driven. The AI-based network engineering enables the complex data mining to understand interactions and causality. Interrogative “omic” based platform robustly infers cellular intelligence. The fact that some of the markers identified in this example have been previously reported to associate with autism validates that this Platform Technology, and the cell models used in the Platform Technology for autism, provide a solid foundation for the identification of biomarkers useful for the diagnosis and patient stratification under the spectrum of autism. The AI-based network engineering approach to data mining employed in the platform technology as a means to infer causality results in actionable biological intelligence. The exemplary autism causal interaction networks for autism shown in FIGS. 12-15 identified several novel biomarkers and potential therapeutic targets for autism. The interrogative discovery platform technology described herein allows for an enhanced understanding of pathophysiology and can thereby drive the identification of therapeutics and biomarkers for pervasive development disorders, including Autism Spectrum Disorder.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method of assessing whether a subject is afflicted with a pervasive developmental disorder, the method comprising:
(1) determining a level of expression of one or more of the markers listed in Tables 2-6 in a biological sample obtained from the subject, using reagents that transform the markers such that the markers can be detected;
(2) comparing the level of expression of the one or more markers in the biological sample obtained from the subject with the level of expression of the one or more markers in a control sample; and
(3) assessing whether the subject is afflicted with a pervasive developmental disorder, wherein a modulation in the level of expression of the one or more markers in the biological sample obtained from the subject relative to the level of expression of the one or more markers in the control sample is an indication that the subject is afflicted with a pervasive developmental disorder.

2-4. (canceled)

5. The method of claim 1, further comprising selecting a treatment regimen for the subject identified as being afflicted with a pervasive developmental disorder or predisposed to developing a pervasive developmental disorder.

6. The method of claim 1, further comprising administering a treatment regimen to the subject identified as being afflicted with a pervasive developmental disorder or predisposed to developing a pervasive developmental disorder.

7. (canceled)

8. A method for assessing the efficacy of a treatment regimen for treating a pervasive developmental disorder or symptoms of a pervasive developmental disorder in a subject, the method comprising:
(1) determining a level of expression of one or more of the markers listed in Tables 2-6 present in a first biological sample obtained from the subject prior to administering at least a portion of the treatment regimen to the subject, using reagents that transform the markers such that the markers can be detected;
(2) determining a level of expression of one or more of the markers listed in Tables 2-6 present in a second biological sample obtained from the subject following administration of at least a portion of the treatment regimen to the subject, using reagents that transform the markers such that the markers can be detected;
(3) comparing the level of expression of one or more markers listed in Tables 2-6 present in the first sample obtained from the subject prior to administering at least a portion of the treatment regimen to the subject with the level of expression of the one or more markers present in the second sample obtained from the subject following administration of at least a portion of the treatment regimen; and
(4) assessing whether the treatment regimen is efficacious for treating the pervasive developmental disorder or symptoms of the pervasive developmental disorder, wherein a modulation in the level of expression of the one or more markers in the second sample as compared to the first sample is an indication that the treatment regimen is efficacious for treating the pervasive developmental disorder or symptoms of the pervasive developmental disorder in the subject.

9. The method of claim 8, further comprising continuing administration of the treatment regimen to the subject for whom the treatment regimen is determined to be efficacious for treating the pervasive developmental disorder or symptoms of the pervasive developmental disorder, or discontinuing administration of the treatment regimen to the subject for whom the treatment regimen is determined to be non-efficacious for treating the pervasive developmental disorder or symptoms of the pervasive developmental disorder.

10. (canceled)

11. The method of claim 1, wherein the pervasive developmental disorder is an autism spectrum disorder.

12. The method of claim 1, wherein the pervasive developmental disorder is autistic disorder.

13. The method of claim 1, wherein the pervasive developmental disorder is Alzheimer's disease.

14. The method of claim 1, wherein the pervasive developmental disorder is autism and Alzheimer's disease.

15. The method of claim 1, wherein the pervasive developmental disorder is Asperger's syndrome.

16. The method of claim 1, wherein the pervasive developmental disorder is a pervasive developmental disorder-not otherwise specified.

17. The method of claim 1, wherein the subject suffers from a pervasive developmental disorder.

18. The method of claim 1, wherein the subject exhibits subsyndromal manifestations of a pervasive developmental disorder.

19. The method of claim 1, wherein the subject is suspected to suffer from or be predisposed to developing a pervasive developmental disorder.

20. The method of claim 1, wherein the level of expression of the one or more markers is determined at a nucleic acid level.

21. The method of claim 20, wherein the level of expression of the one or more markers is determined by detecting RNA.

22. The method of claim 20, wherein the level of expression of the one or more markers is determined by detecting mRNA, miRNA, or hnRNA.

23. The method of claim 20, wherein the level of expression of the one or more markers is determined by detecting DNA.

24. The method of claim 20, wherein the level of expression of the one or more markers is determined by detecting cDNA.

25. The method of claim 20, wherein the level of expression of the one or more markers is determined by using a technique selected from the group consisting of a polymerase chain reaction (PCR) amplification reaction, reverse-transcriptase PCR analysis, quantitative reverse-transcriptase PCR analysis, Northern blot analysis, an RNAase protection assay, digital RNA detection/quantitation, and a combination or sub-combination thereof.

26-47. (canceled)
